(19) United States
(12) Patent Application Publication
(10) Pub. No.: US 2023/0398161 A1
(43) Pub. Date: Dec. 14, 2023

(54) PHAGE COMPOSITIONS FOR ESCHERICHIA COMPRISING CRISPR-CAS SYSTEMS AND METHODS OF USE THEREOF

(71) Applicant: Locus Biosciences, Inc., Morrisville, NC (US)

(72) Inventors: Paul GAROFOLO, Morrisville, NC (US); David G. OUSTEROUT, Morrisville, NC (US); Kurt SELLE, Raleigh, NC (US); Hannah Hewitt TUSON, Morrisville, NC (US); Lana MCMILLAN, Morrisville, NC (US); Drew DELORENZO, Morrisville, NC (US); Robert MCKEE, Morrisville, NC (US); Cameron PRYBOL, Morrisville, NC (US)

(21) Appl. No.: 18/033,988

(22) PCT Filed: Nov. 4, 2021
(86) PCT No.: PCT/US21/58095

§ 371 Date: Apr. 26, 2023

Related U.S. Application Data
(60) Provisional application No. 63/110,107, filed on Nov. 5, 2020.
(60) Provisional application No. 63/184,647, filed on May 5, 2021.

(51) Int. Cl. A61K 35/76; C12N 7/00; C12N 15/11; C12N 9/22

(52) U.S. Cl. CPC A61K 35/76; C12N 7/00; C12N 15/11; C12N 9/22; C12N 2310/20; C12N 2795/10021; C12N 2795/10071; C12N 2795/10032

(57) ABSTRACT

Disclosed here are phage compositions for Escherichia comprising CRISPR-Cas systems and methods of use thereof.

### CROSS-REFERENCE

This application claims the benefit of U.S. Provisional Application No. 63/110,107, filed on Nov. 5, 2020, and U.S. Provisional Application No. 63/184,647, filed on May 5, 2021, both of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 3, 2021, is named 53240-744_601_SL.txt and is 43,562 bytes in size.

### SUMMARY

In some aspects, provided herein are nucleic acids, CRISPR arrays, and bacteriophage comprising the same. Further provided are methods of killing Escherichia with compositions comprising the nucleic acids, CRISPR arrays, and/or bacteriophage.

In certain aspects, provided is a nucleic acid sequence having at least 80% identity to SEQ ID NO: 39. In some embodiments, the nucleic acid comprises SEQ ID NO: 25. In some embodiments, the nucleic acid comprises SEQ ID NO: 24. In some embodiments, the nucleic acid comprises SEQ ID NO: 12. In some embodiments, the nucleic acid comprises SEQ ID NO: 13. In some embodiments, the nucleic acid comprises SEQ ID NO: 39, or a sequence at least 90% identical to SEQ ID NO: 39.

In certain aspects, provided herein is a CRISPR array comprising a nucleic acid sequence herein. In some embodiments, the CRISPR array comprises a promoter. In some embodiments, the promoter comprises a sequence at least about 80%, identical to any one of SEQ ID NOS: 11, 1-10 or 19. In some embodiments, the promoter comprises a sequence at least about 80%, identical to SEQ ID NO: 11. In some embodiments, the CRISPR array comprises a sequence at least 80% identical to SEQ ID NO: 43.

In certain aspects, provided herein is a CRISPR array comprising a sequence at least 80% identical to SEQ ID NO: 43.

In certain aspects, provided herein is a bacteriophage comprising a nucleic acid or CRISPR array herein. In some embodiments, the nucleic acid and/or CRISPR array replaces bacteriophage DNA. In some embodiments, the bacteriophage DNA is from a p004ke, p00c0, or p00ex phage. In some embodiments, the nucleic acid and/or CRISPR array is part of a CRISPR system present in the bacteriophage. In some embodiments, the CRISPR system comprises a total of about 3000 nucleobases to about 8000 nucleobases. In some embodiments, the CRISPR system comprises a Type I CRISPR-Cas system, a Type II CRISPR-Cas system, a Type V CRISPR-Cas system, or a CRISPR-Cpf1 system.

In certain aspects, provided herein are recombinant phage. In some embodiments, provided is a recombinant phage comprising at least 80% sequence identity to p004ke009. In some embodiments, provided is a recombinant phage comprising at least 80% sequence identity to p00c0e030. In some embodiments, provided is a recombinant phage comprising at least 80% sequence identity to p00exe014. In some embodiments, provided is a recombinant phage comprising at least 90% sequence identity to p004ke009. In some embodiments, provided is a recombinant phage comprising at least 90% sequence identity to p00c0e030. In some embodiments, provided is a recombinant phage comprising at least 90% sequence identity to p00exe014.

In certain aspects, provided herein is a bacteriophage comprising a CRISPR system comprising: (a) a CRISPR array comprising a first spacer sequence comprising a sequence selected from SEQ ID NOS: 12 or 20-37; and (b) a nucleic acid sequence encoding a CRISPR associated nuclease. In some embodiments, the first spacer sequence comprises SEQ ID NO: 12, SEQ ID NO: 25, or SEQ ID NO: 24. In some embodiments, the CRISPR array comprises a second spacer sequence. In some embodiments, the first spacer sequence comprises SEQ ID NO: 12, and the second spacer sequence comprises SEQ ID NO: 25, or the first spacer sequence comprises SEQ ID NO: 12, and the second spacer sequence comprises SEQ ID NO: 24, or the first spacer sequence comprises SEQ ID NO: 25, and the second spacer sequence comprises SEQ ID NO: 24. In some embodiments, the CRISPR array comprises a third spacer sequence. In some embodiments, the first spacer sequence comprises SEQ ID NO: 12, the second spacer sequence comprises SEQ ID NO: 25, and the third spacer sequence comprises SEQ ID NO: 24.

In certain aspects, provided herein is a bacteriophage comprising a CRISPR system comprising: (a) a CRISPR array comprising a first spacer sequence and a second spacer sequence, wherein the first spacer sequence is complementary to a first target nucleotide sequence in a first Escherichia species, and the second spacer sequence is complementary to a second target nucleotide sequence in the first Escherichia species and/or a second Escherichia species; optionally further comprising a third spacer sequence complementary to a third target nucleotide sequence in the Escherichia species, second Escherichia species, and/or a third Escherichia species; and (b) a nucleic acid sequence encoding a CRISPR associated nuclease. In some embodiments, the first spacer sequence comprises a sequence at least about 90%, sequence identity to any one of SEQ ID NOS: 12 or 20-37, wherein the second spacer sequence comprises a sequence at least about 90%, sequence identity to any one of SEQ ID NOS: 12 or 20-37, and if present, the third spacer sequence comprises a sequence at least about 90%, sequence identity to any one of SEQ ID NOS: 12 or 20-37.

In some embodiments, the CRISPR array comprises a repeat sequence comprising a sequence with at least about 90% sequence identity to any one of SEQ ID NOs: 13-18. In some embodiments, the CRISPR array comprises a promoter sequence comprising a sequence at least about 90% identical to any one of SEQ ID NOs: 11, 1-10 or 19. In some embodiments, the CRISPR system comprises a Type I CRISPR-Cas system, a CRISPR-Cpf1 system, a Type II CRISPR-Cas system, or a Type V CRISPR-Cas system.

In certain aspects, provided herein is a bacteriophage modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease, wherein the target bacteria comprises an Escherichia species. In some embodiments, the CRISPR system comprises a total of about 3000 nucleobases to about 8000 nucleobases. In some embodiments, the bacteriophage DNA is not essential for viability or functionality of the bacteriophage. In some embodiments, the bacteriophage DNA is from a p004ke, p00c0, or p00ex phage. In some embodiments, the CRISPR array comprises a spacer sequence comprising a sequence having at least about 90% sequence identity to any one of SEQ ID NOS: 12 or 20-37. In some embodiments, the CRISPR array comprises a repeat sequence comprising at least about 90% identity to any one of SEQ ID NOs: 13-18. In some embodiments, the CRISPR array comprises a promoter sequence comprises at least about 90% identity to any one of SEQ ID NOs: 11, 1-10 or 19. In some embodiments, the CRISPR system comprises a Type I CRISPR-Cas system, a CRISPR-Cpf1 system, a Type II CRISPR-Cas system, or a Type V CRISPR-Cas system.

In certain aspects, provided herein is a composition comprising a bacteriophage herein and a wild-type phage. In some embodiments, the wild-type phage comprises p00ke. In some embodiments, the wild-type phage comprises p5516. In some embodiments, the wild-type phage comprises p00jc.

In certain aspects, provided herein is a composition comprising two or more bacteriophage. In some embodiments, provided is a composition comprising a first phage having at least 80% sequence identity to p004ke009, and a second phage having at least 80% sequence identity to p00c0e030, p00exe014, p00jc, p00ke, or p5516. In some embodiments, provided is a composition comprising a first phage having at least 80% sequence identity to p00c0e030, and a second phage having at least 80% sequence identity to p004ke009, p00exe014, p00jc, p00ke, or p5516. In some embodiments, provided is a composition comprising a first phage having at least 80% sequence identity to p00exe014, and a second phage having at least 80% sequence identity to p004ke009, p00c0e030, p00jc, p00ke, or p5516. In some embodiments, provided is a composition comprising a first phage having at least 80% sequence identity to p00jc, and a second phage having at least 80% sequence identity to p004ke009, p00c0e030, p00exe014, p00ke, or p5516. In some embodiments, provided is a composition comprising a first phage having at least 80% sequence identity to p00ke, and a second phage having at least 80% sequence identity to p004ke009, p00c0e030, p00exe014, p00jc, or p5516. In some embodiments, provided is a composition comprising a first phage having at least 80% sequence identity to p5516, and a second phage having at least 80% sequence identity to p004ke009, p00c0e030, p00exe014, p00jc, or p00ke.

In certain aspects, provided herein is a pharmaceutical composition comprising: (a) the bacteriophage of any one of claims 1-44 or the composition of any one of claims 45-54; and (b) a pharmaceutically acceptable excipient. In some embodiments, the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.

In certain aspects, provided herein are methods of bacterial killing and methods of treatment. In some embodiments, provided is a method of killing bacteria comprising introducing into the bacteria genetic material from a bacteriophage or composition herein. In some embodiments, provided is a method of treating a disease in an individual in need thereof, the method comprising administering to the individual a bacteriophage or composition herein. In some embodiments, provided is a method of killing bacteria comprising contacting the bacteria with a composition comprising a bacteriophage at least 80% identity to p00jc. In some embodiments, provided is a method of killing bacteria comprising contacting the bacteria with a composition comprising a bacteriophage at least 80% identity to p00ke. In some embodiments, provided is a method of killing bacteria comprising contacting the bacteria with a composition comprising a bacteriophage at least 80% identity to p5516. In some embodiments, provided is a method of treating a disease in an individual in need thereof, the method comprising administering to the individual a composition comprising a bacteriophage at least 80% identity to p00jc. In some embodiments, provided is a method of treating a disease in an individual in need thereof, the method comprising administering to the individual a composition comprising a bacteriophage at least 80% identity to p00ke. In some embodiments, provided is a method of treating a disease in an individual in need thereof, the method comprising administering to the individual a composition comprising a bacteriophage at least 80% identity to p5516. In some embodiments, provided is a method of killing a plurality of bacteria, the method comprising combining the plurality of bacteria with a first bacteriophage and a second bacteriophage, wherein the first bacteriophage targets a first subset of the plurality of bacteria, and the second bacteriophage targets a second subset of the plurality of bacteria, wherein the plurality of bacteria comprises two or more bacteria of Table 6. In some embodiments, provided is a method of treating a disease in an individual comprising a plurality of bacteria, the method comprising administering to the individual a first bacteriophage and a second bacteriophage, wherein the first bacteriophage targets a first subset of the plurality of bacteria, and a second bacteriophage targets the second subset of the plurality of bacteria, wherein the plurality of bacteria comprises two more bacteria of Table 6. In some embodiments, the plurality of bacteria comprises at least 50, 100, 150, 200, 250, 300, or 350 bacteria of Table 6. In some embodiments, the first and/or second bacteriophage comprises a bacteriophage herein. In some embodiments, (i) the first bacteriophage comprises a phage at least 80% identical to p00jc, (ii) the first bacteriophage comprises a phage at least 80% identical to p00ke, (iii) the first bacteriophage comprises a phage at least 80% identical to p5516, (iv) the first bacteriophage comprises a phage at least 80% identical to p004Ke009, (v) the first bacteriophage comprises a phage at least 80% identical to p00c0e030, or (vi) the first bacteriophage comprises a phage at least 80% identical to p00exe014. In some embodiments, (i) the first phage has at least 80% sequence identity to p004ke009, and the second phage has at least 80% sequence identity to p00c0e030, p00exe014, p00jc, p00ke, or p5516, (ii) the first phage has at least 80% sequence identity to p00c0e030, and the second phage has at least 80% sequence identity to p004ke009, p00exe014, p00jc, p00ke, or p5516, (iii) the first phage has at least 80% sequence identity to p00exe014, and the second phage has at least 80% sequence identity to p004ke009, p00c0e030, p00jc, p00ke, or p5516, (iv) the first phage has at least 80% sequence identity to p00jc, and the second phage has at least 80% sequence identity to p004ke009, p00c0e030, p00exe014, p00ke, or p5516, (v) the first phage has at least 80% sequence identity to p00ke, and the second phage has at least 80% sequence identity to p004ke009, p00c0e030, p00exe014, p00jc, or p5516, or (vi) the first phage has at least 80% sequence identity to p5516, and the second phage has at least 80% sequence identity to p004ke009, p00c0e030, p00exe014, p00jc, or p00ke.

In certain aspects, disclosed herein is a bacteriophage comprising a nucleic acid sequence encoding a Type I CRISPR-Cas system comprising: a CRISPR array comprising a spacer sequence complementary to target nucleotide sequence in an Escherichia species; a Cascade polypeptide; and a Cas3 polypeptide. In some embodiments, the spacer sequence comprises at least about 90% sequence identity to any one of SEQ ID NOS: 12 or 20-37. In some embodiments, the CRISPR array further comprises at least one repeat sequence. In some embodiments, the at least one repeat sequence is operably linked to the spacer sequence at either its 5′ end or its 3′ end. In some embodiments, the repeat sequence comprises at least about 90% sequence identity to any one of SEQ ID NOs: 13-18. In some embodiments, the CRISPR array comprises at least about 90% sequence identity to a sequence as set forth in any one of FIGS. 1A-1C. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the promoter sequence comprises at least about 90% sequence identity to any one of SEQ ID NOs: 1-11 or 19. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. In some embodiments, the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). In some embodiments, the Cascade complex comprises a Cas5d polypeptide, a Cas8c polypeptide, and a Cas7 polypeptide (Type I-C CRISPR-Cas system). In some embodiments, the Cascade complex comprises a Pseudomonas aeruginosa Type I-C Cascade complex. In some embodiments, the nucleic acid sequence further comprises a promoter sequence. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the Escherichia species is killed solely by lytic activity of the bacteriophage. In some embodiments, the Escherichia species is killed solely by activity of the CRISPR-Cas system. In some embodiments, the Escherichia species is killed by lytic activity of the bacteriophage in combination with activity of the CRISPR-Cas system. In some embodiments, the Escherichia species is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage. In some embodiments, the bacteriophage comprises a Tequatrovirus, a Mosigyvirus, a Phapecoctavirus, a Unique Myoviridae, or a Vectrevirus. In some embodiments, the bacteriophage comprises at least 80% sequence identity to a phage selected from p004K, p00c0, p00ex, p00jc, p00ke, or p5516. In some embodiments, the bacteriophage is p004ke007 or p004Ke005. In some embodiments, the nucleic acid sequence is inserted into a non-essential bacteriophage gene. In some embodiments, disclosed herein is a pharmaceutical composition comprising: (a) the bacteriophage disclosed herein; and (b) a pharmaceutically acceptable excipient. In some embodiments, the pharmaceutical composition comprises at least two bacteriophage. In some embodiments, the bacteriophage are from the lineage consisting of a Tequatrovirus, a Mosigyvirus, a Phapecoctavirus, a Unique Myoviridae, or a Vectrevirus. In some embodiments, the pharmaceutical composition comprises at least six bacteriophage, wherein the bacteriophage comprise p004k, p00c0, p00ex, p00jc, p00ke, and p5516. In some embodiments, the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.

In certain aspects, disclosed herein is a method of killing an Escherichia species comprising introducing into the Escherichia species a bacteriophage comprising a nucleic acid sequence encoding a Type I CRISPR-Cas system comprising: a CRISPR array comprising a spacer sequence complementary to target nucleotide sequence in the Escherichia species; a Cascade polypeptide; and a Cas3 polypeptide. In some embodiments, the spacer sequence comprises at least about 90% sequence identity to SEQ ID NO: 12 or 20-37. In some embodiments, the CRISPR array further comprises at least one repeat sequence. In some embodiments, the at least one repeat sequence is operably linked to the spacer sequence at either its 5′ end or its 3′ end. In some embodiments, the repeat sequence comprises at least about 90% sequence identity to any one of SEQ ID NOs: 13-18. In some embodiments, the CRISPR array comprises at least about 90% sequence identity to a sequence as set forth in any one of FIGS. 1A-1C. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the promoter sequence comprises at least about 90% sequence identity to any one of SEQ ID NOs: 1-11 or 19. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. In some embodiments, the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). In some embodiments, the Cascade complex comprises a Cas5d polypeptide, a Cas8c polypeptide, and a Cas7 polypeptide (Type I-C CRISPR-Cas system). In some embodiments, the Cascade complex comprises a Pseudomonas aeruginosa Type I-C Cascade complex. In some embodiments, the nucleic acid sequence further comprises a promoter sequence. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the Escherichia species is killed solely by activity of the CRISPR-Cas system. In some embodiments, the Escherichia species is killed by lytic activity of the bacteriophage in combination with activity of the CRISPR-Cas system. In some embodiments, the Escherichia species is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage. In some embodiments, the bacteriophage infects multiple bacterial strains. In some embodiments, the bacteriophage comprises at least 80% identity to p004ke007, p004Ke005, p004K, p00c0, p00ex, p00jc, p00ke, or p5516. In some embodiments, the nucleic acid sequence is inserted in pace of or adjacent to a non-essential bacteriophage gene. In some embodiments, a mixed population of bacterial cells comprises the Escherichia species.

In certain aspects, disclosed herein is a method of treating a disease in an individual in need thereof, the method comprising administering to the individual a bacteriophage comprising a nucleic acid sequence encoding a Type I CRISPR-Cas system comprising: a CRISPR array; a Cascade polypeptide comprising a spacer sequence complementary to target nucleotide sequence in an Escherichia species; and a Cas3 polypeptide. In some embodiments, the spacer sequence comprises at least about 90% sequence identity to SEQ ID NO: 12 or 20-37. In some embodiments, the CRISPR array further comprises at least one repeat sequence. In some embodiments, the at least one repeat sequence is operably linked to the spacer sequence at either its 5′ end or its 3′ end. In some embodiments, the repeat sequence comprises at least about 90% sequence identity to any one of SEQ ID NOs: 13-18. In some embodiments, the CRISPR array comprises at least about 90% sequence identity to a sequence as set forth in any one of FIGS. 1A-1C. In some embodiments, the target nucleotide sequence comprises a coding sequence. In some embodiments, the target nucleotide sequence comprises a non-coding or intergenic sequence. In some embodiments, the target nucleic acid sequence comprises all or a part of a promoter sequence. In some embodiments, the promoter sequence comprises at least about 90% sequence identity to any one of SEQ ID NOs: 1-11 or 19. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. In some embodiments, the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); (vi) a Csy1 polypeptide, a Csy2 polypeptide, a Csy3 polypeptide, and a Csy4 polypeptide (Type I-F CRISPR-Cas system). In some embodiments, the Cascade complex comprises a Cas5d polypeptide, a Cas8c polypeptide, and a Cas7 polypeptide (Type I-C CRISPR-Cas system). In some embodiments, the nucleic acid sequence further comprises a promoter sequence. In some embodiments, the Escherichia species is killed solely by activity of the CRISPR-Cas system. In some embodiments, the Escherichia species is killed by lytic activity of the bacteriophage in combination with activity of the CRISPR-Cas system. In some embodiments, the Escherichia species is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the bacteriophage comprises at least 80% identity to p004ke007, p004Ke005, p004K, p00c0, p00ex, p00jc, p00ke, or p5516. In some embodiments, the nucleic acid sequence is inserted in place of or adjacent to a non-essential bacteriophage gene. In some embodiments, the disease is a bacterial infection. In some embodiments, the Escherichia species causing the disease is a drug resistant Escherichia species. In some embodiments, the drug resistant Escherichia species is resistant to at least one antibiotic. In some embodiments, the Escherichia species causing the disease is a multidrug resistant Escherichia species. In some embodiments, the multi-drug resistant Escherichia species is resistant to at least one antibiotic. In some embodiments, the antibiotic comprises a cephalosporin, a fluoroquinolone, a carbapenem, a colistin, an aminoglycoside, vancomycin, streptomycin, or methicillin. In some embodiments, the Escherichia species is Escherichia coli. In some embodiments, the administering is intra-arterial, intravenous, intraurethral, intramuscular, oral, subcutaneous, inhalation, topical, cutaneous, transdermal, transmucosal, implantation, sublingual, buccal, rectal, vaginal, ocular, otic, or nasal administration or any combination thereof. In some embodiments, the individual is a mammal. In some embodiments, the spacer sequence comprises at least about 90% sequence identity to SEQ ID NO: 12 or 20-37 In some embodiments, the CRISPR array further comprises at least one repeat sequence. In some embodiments, the at least one repeat sequence is operably linked to the spacer sequence at either its 5′ end or its 3′ end. In some embodiments, the repeat sequence comprises at least about 90% sequence identity to any one of SEQ ID NOs: 13-18. In some embodiments, the CRISPR array comprises at least about 90% sequence identity to a sequence as set forth in any one of FIGS. 1A-1C. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the promoter sequence comprises at least about 90% sequence identity to any one of SEQ ID NOs: 1-11 or 19. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the nucleic acid sequence further comprises a promoter sequence. In some embodiments, the Escherichia species is killed solely by lytic activity of the bacteriophage. In some embodiments, the Escherichia species is killed solely by activity of the CRISPR-Cas system. In some embodiments, the Escherichia species is killed by lytic activity of the bacteriophage in combination with activity of the CRISPR-Cas system. In some embodiments, the Escherichia species is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of a lysogeny gene. In some embodiments, the bacteriophage comprises at least 80% identity to p004ke007, p004Ke005, p004K, p00c0, p00ex, p00jc, p00ke, or p5516. In some embodiments, the nucleic acid sequence is inserted into a non-essential bacteriophage gene.

In some embodiments, the mature crRNA is bound by a single Cas protein in an effector complex. In some embodiments, the CRISPR-Cas complex is a Type II system. In some embodiments, the Type II effector complex comprising a spacer sequence comprises a sequence complementary to target nucleotide sequence in an Escherichia species; and a Cas9 polypeptide. In some embodiments, the spacer sequence comprises at least about 90% sequence identity to SEQ ID NO: 12 or 20-37. In some embodiments, the CRISPR array further comprises at least one repeat sequence. In some embodiments, the at least one repeat sequence is operably linked to the spacer sequence at either its 5′ end or its 3′ end. In some embodiments, the repeat sequence comprises at least about 90% sequence identity to any one of SEQ ID NOs: 13-18. In some embodiments, the target nucleotide sequence comprises all or a part of a nucleotide sequence located on a coding strand of a transcribed region of an essential gene. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the nucleic acid sequence further comprises a promoter sequence. In some embodiments, the Escherichia species is killed solely by lytic activity of the bacteriophage. In some embodiments, the Escherichia species is killed solely by activity of the CRISPR-Cas system. In some embodiments, the Escherichia species is killed by lytic activity of the bacteriophage in combination with activity of the CRISPR-Cas system. In some embodiments, the Escherichia species is killed by the activity of the CRISPR-Cas system, independently of the lytic activity of the bacteriophage. In some embodiments, the activity of the CRISPR-Cas system supplements or enhances the lytic activity of the bacteriophage. In some embodiments, the lytic activity of the bacteriophage and the activity of the CRISPR-Cas system are synergistic. In some embodiments, the lytic activity of the bacteriophage, the activity of the CRISPR-Cas system, or both, is modulated by a concentration of the bacteriophage.

In some embodiments, the CRISPR-Cas system is a Type III system, wherein the mature crRNA is bound by Cas10 and other Cas proteins.

In some embodiments, the CRISPR-Cas system is a Type V system. In some embodiments, the CRISPR-Cas system is bound by a Cas12 protein.

In some embodiments, disclosed herein is a pharmaceutical composition comprising: (a) the bacteriophage disclosed herein; and (b) a pharmaceutically acceptable excipient. In some embodiments, the pharmaceutical composition comprises at least two bacteriophage. In some embodiments, the pharmaceutical composition comprises at last six bacteriophage, wherein each bacteriophage comprises at least 80% sequence identity to p004k, p00c0, p00ex, p00jc, p00ke, and p5516. In some embodiments, the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.

In certain aspects, disclosed herein is a method of sanitizing a surface in need thereof, the method comprising administering to the surface a bacteriophage comprising a nucleic acid sequence encoding a Type I CRISPR-Cas system comprising: a CRISPR array; a Cascade polypeptide comprising a spacer sequence complementary to target nucleotide sequence in an Escherichia species; and a Cas3 polypeptide. In some embodiments, the surface is a hospital surface, a vehicle surface, an equipment surface, or an industrial surface.

In certain aspects, disclosed herein is a method of preventing contamination in a food product or a nutritional supplement, the method comprising adding to the food product or the nutritional supplement a bacteriophage comprising a nucleic acid sequence encoding a Type I CRISPR-Cas system comprising: a CRISPR array; a Cascade polypeptide comprising a spacer sequence complementary to target nucleotide sequence in an Escherichia species; and a Cas3 polypeptide. In some embodiments, the food product or nutritional supplement comprises milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

In certain aspects, disclosed herein is a bacteriophage comprising a nucleic acid sequence encoding a Type I CRISPR-Cas system comprising: a CRISPR array comprising spacer sequences complementary to target nucleotide sequence in a Escherichia species, wherein the spacer sequences comprise SEQ ID NOs 12 or 20-37; a Cascade polypeptide; and a Cas3 polypeptide.

In certain aspects, disclosed herein is a bacteriophage comprising at least 80% sequence identity to a phage selected from p004K, p00c0, p00ex, p00jc, p00ke, or p5516. In some embodiments, the bacteriophage comprises at least 80% identity to p004ke007 or p004Ke005. In some embodiments, the bacteriophage further comprises a CRISPR array; a Cascade polypeptide comprising one or more spacer sequences complementary to target nucleotide sequence in a Pseudomonas species; and a Cas3 polypeptide. In some embodiments, the one or more spacer sequence comprises at least one of SEQ ID NOs: 12 or 20-37 or at least 90% sequence identity to any one of SEQ ID NOs: 12 or 20-37. In some embodiments, the spacer sequence comprises at least about 90% sequence identity to SEQ ID NO: 12 or 20-37. In some embodiments, the CRISPR array further comprises at least one repeat sequence. In some embodiments, the at least one repeat sequence is operably linked to the spacer sequence at either its 5′ end or its 3′ end. In some embodiments, the repeat sequence comprises at least about 90% sequence identity to any one of SEQ ID NOs: 13-18. In some embodiments, the CRISPR array comprises at least about 90% sequence identity to a sequence as set forth in any one of FIGS. 1A-1C. In some embodiments, the target nucleotide sequence comprises a coding sequence. In some embodiments, the target nucleotide sequence comprises a non-coding or intergenic sequence. In some embodiments, the target nucleotide sequence comprises all or a part of a promoter sequence. In some embodiments, the promoter sequence comprises at least about 90% sequence identity to any one of SEQ ID NOs: 1-11 or 19.

### BRIEF DESCRIPTION OF THE DRAWINGS

The novel features of the disclosures are set forth with particularity in the appended claims. A better understanding of the features and advantages of the present disclosures will be obtained by reference to the following detailed description that sets forth illustrative embodiments, in which the principles of the disclosures are utilized, and the accompanying drawings of which:

FIG. 1A depicts the sequence and arrangement of the crArray containing the SC2 spacer (SEQ ID NO: 42, with linker SEQ ID NO: 55).

FIG. 1B depicts the array sequence for phages p004Ke009, p00c0e030, and p00exe014 (labeled PAIC array 2, SEQ ID NO: 43).

FIG. 1C depicts the pJC_Cfp1 array sequence (array: SEQ ID NO: 44, sequence shown: SEQ ID NO: 56).

FIG. 2 is a schematic representation of the genome of wild type phage p004k and its engineered variant p004ke007. The bar below the genome axis indicates the region of the genome that was removed and replaced. The schematic below the phage genome illustrates the DNA that was used to replace WT phage genes in the deleted region.

FIGS. 3A-3D illustrates the efficacy of the crArray/Cas system insert in E. coli. FIG. 3A depicts the growth on an agar plate of p004kwt (wild type) and p004ke007 (SC2+Cas system) mixed with three strains of E. coli (b3402, b3418, or b4098). FIGS. 3B-3D are a quantification of the optical density of the cell growth in b3402, b3418, and b4098, respectively.

FIG. 4 depicts the growth on an agar plate of p00EX.015 and p00EXe014.008 (3 spacer full construct) mixed with two strains of E. coli (b2185 or b3911).

FIG. 5 shows an exemplary CRISPR-Cas system for testing selected constructs.

FIG. 6 illustrates the plasmid based experimental set up to test crRNA array in targeting bacteria.

FIG. 7 shows data demonstrating that the spacers have high coverage and efficacy.

FIG. 8 shows data from individual PAIC spacer kill test.

FIG. 9A shows crRNA alone causes no statistical reduction in CFU formation.

FIGS. 9B-9C shows data indicating selected spacers do not activate endogenous EcIF or EcIE systems. FIG. 9B shows ECIF crosstalk of the identified spacers. FIG. 9C shows ECIE activity of PAIC spacers.

FIG. 10 shows a mutational model for predicting number of targeting sites for mitigating mutational escape.

FIG. 11 shows plasmid-based PAIC array kill data.

FIG. 12A-12C shows host range of CK618 and antibiotics on 88-isolate panel. FIG. 12A, Host range on all 88 strains. FIG. 12B, Host range on 21 MDR strains. FIG. 12C, Host range on 31 strains with any beta-lactam resistance excluding cephems. Single treatment is treatment with either CK618 or antibiotic. Antibiotic+CK618 is the combination of antibiotic and CK618.

FIG. 13A-13C shows host range of CK618 and antibiotics on 304-isolate panel. FIG. 13A, Host range on all 304 strains. FIG. 13B, Host range on 93 MDR strains. FIG. 13C, Host range on 91 strains with any beta-lactam resistance excluding cephems. Single treatment is treatment with either CK618 or antibiotic. Antibiotic+CK618 is the combination of antibiotic and CK618.

FIG. 14A shows the schematic design of in vivo testing of the engineered phage efficacy using CK570 phage cocktail.

FIG. 14B shows the schematic design of in vivo testing of the engineered phage efficacy using CK618 phage cocktail.

FIGS. 15A-15C show data demonstrating reduction in bacterial burden in kidney (FIG. 15A), bladder (FIG. 15B) and urine (FIG. 15C) following CK570 administration in mouse UTI model.

FIGS. 16A-16E show data demonstrating detectable levels of phage in kidney (FIG. 16A), bladder (FIG. 16B), urine (FIG. 16C), blood (FIG. 16D) and spleen (FIG. 16E) following CK570 administration.

FIGS. 17A-17B show data demonstrating reduction in bacterial burden in kidney (FIG. 17A), and bladder (FIG. 17B) following CK618 administration in mouse UTI model.

FIGS. 18A-18C show data demonstrating biodistribution of intravenous CK618 administered either as single dose or as five twice daily doses, with or without an initial intraurethral dose of CK618, in kidney (FIG. 18A), bladder (FIG. 18B), and blood plasma (FIG. 18C).

FIG. 19 shows graphical representations of AUC (area under the curve) ratio and time to OD (optical density).

### DETAILED DESCRIPTION

Disclosed herein, in certain embodiments, are bacteriophages comprising a nucleic acid sequence encoding a CRISPR system. In some embodiments, the CRISPR system comprises a CRISPR array comprising a spacer sequence complementary to a target nucleotide sequence in an Escherichia species, and a nucleic acid sequence encoding a CRISPR associated nuclease. An example CRISPR system is a Type I CRISPR-Cas system comprising: (a) a CRISPR array (also referred to as “crArray”); (b) a Cascade polypeptide; and (c) a Cas3 polypeptide. Another example is the CRISPR-Cpf1 system. Further example CRISPR systems include Type II and Type V CRISPR-Cas systems.

Additional bacteriophage embodiments disclosed herein include a bacteriophage modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease.

Also disclosed herein, in certain embodiments, are pharmaceutical compositions comprising the bacteriophages disclosed herein.

Further disclosed herein, in certain embodiments, are methods of killing a Escherichia species comprising introducing into the Escherichia species a bacteriophage described herein. Further disclosed herein, in certain embodiments, are methods of treating a disease in an individual in need thereof, the method comprising administering to the individual a bacteriophage described herein. The bacteriophage in some embodiments is a wild-type phage. The bacteriophage in some embodiments is an engineered phage. The bacteriophage in some embodiments comprises one or more bacteriophage of Table 1, or at least 80% sequence identity to one or more bacteriophage of Table 1. The bacteriophage may comprise two or more different bacteriophage in a cocktail, e.g., CK618.

The present disclosure further provides nucleic acid sequences, e.g., for incorporation into a bacteriophage. In some embodiments, the nucleic acid sequence has at least 80% identity to any one of SEQ ID NOS: 38-44. Some such nucleic acid sequences comprise a spacer and/or a repeat sequence, wherein the spacer sequence if present is complementary to a target bacteria sequence.

### Certain Terminology

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, +0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

In some embodiments, as used herein “a part of” or “a portion of” or similar language includes at least 10 contiguous nucleobases or amino acids, as applicable.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

“Complement” as used herein means 100% complementarity or identity with the comparator nucleotide sequence or it means less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

As used herein, the term “CRISPR phage”, “CRISPR enhanced phage”, and “crPhage” refers to a bacteriophage particle comprising bacteriophage DNA comprising at least one heterologous polynucleotide that encodes at least one component of a CRISPR-Cas system (e.g., CRISPR array, crRNA; e.g., P1 bacteriophage comprising an insertion of a targeting crRNA). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refers to two or more sequences or subsequences that have at least about 70%, at least about 75%, at least about 80%, at least about 85%, at least about 90%, at least about 95, 96, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

As used herein, a “target nucleotide sequence” refers to the portion of a target gene (i.e., target region in the genome or the “protospacer sequence,” which is adjacent to a protospacer adjacent motif (PAM) sequence) that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array.

As used herein, the term “protospacer adjacent motif” or “PAM” refers to a DNA sequence present on the target DNA molecule adjacent to the nucleotide sequence matching the spacer sequence. This motif is found in the target gene next to the region to which a spacer sequence binds as a result of being complementary to that region and identifies the point at which base pairing with the spacer nucleotide sequence begins. The exact PAM sequence that is required varies between each different CRISPR-Cas system. Non-limiting examples of PAMs include CCA, CCT, CCG, TTC, AAG, AGG, ATG, GAG, and/or CC. In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the P. aeruginosa Type I-C system, the PAM is TTC. Once a cognate protospacer and PAM are recognized, Cas3 is recruited, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a -protospacer adjacent motif recognition domain at the C-terminus of Cas9).

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or on a subject. In some embodiments, the bacterial population comprises one or more target bacterial species. In some embodiments, the one or more bacteria species in the bacterial population comprise one or more strains of one or more bacteria. In some embodiments, the target bacterial population causes an “infection”, “a disease”, or “a condition” that is acute or chronic. In some embodiments, the target bacterial population causes an “infection”, “a disease”, or “a condition” that is localized or systemic. In some embodiments, the target bacterial population causes an “infection”, “a disease”, or “a condition” that is idiopathic. In some embodiments, the target bacterial population causes an “infection”, “a disease”, or “a condition” that is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

The terms “individual”, or “subject” as used herein includes any animal that has or is susceptible to an infection, disease or condition involving bacteria. Thus, in some embodiments, subjects are mammals, avians, reptiles, amphibians, fish, crustaceans, or mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab and the like). Mollusk subjects include but are not limited to species used in aquaculture (e.g., abalone, mussel, oyster, clams, scallop and the like). In some embodiments, suitable subjects include both males and females and subjects of any age, including embryonic (e.g., in-utero or in-ovo), infant, juvenile, adolescent, adult and geriatric subjects. In some embodiments, a subject is a human.

As used here the term “isolated” in context of a nucleic acid sequence is a nucleic acid sequence that exists apart from its native environment.

As used herein, “expression cassette” means a recombinant nucleic acid molecule comprising a nucleotide sequence of interest (e.g., the recombinant nucleic acid molecules and CRISPR arrays disclosed herein), wherein the nucleotide sequence is operably associated with at least a control sequence (e.g., a promoter).

As used herein, “chimeric” refers to a nucleic acid molecule or a polypeptide in which at least two components are derived from different sources (e.g., different organisms, different coding regions).

As used herein, “selectable marker” means a nucleotide sequence that when expressed imparts a distinct phenotype to the host cell expressing the marker and thus allows such transformed cells to be distinguished from those that do not have the marker.

As used herein, “vector” refers to a composition for transferring, delivering or introducing a nucleic acid (or nucleic acids) into a cell.

As used herein, “pharmaceutically acceptable” means a material that is not biologically or otherwise undesirable, i.e., the material are administered to a subject without causing any undesirable biological effects such as toxicity.

As used herein the term “biofilm” means an accumulation of microorganisms embedded in a matrix of polysaccharide. Biofilms form on solid biological or non-biological surfaces and are medically important, accounting for over 80 percent of microbial infections in the body.

As used herein, the term “in vivo” is used to describe an event that takes place in a subject's body.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

### CRISPR/CAS Systems

CRISPR-Cas systems are naturally adaptive immune systems found in bacteria and archaea. The CRISPR system is a nuclease system involved in defense against invading phages and plasmids that provides a form of acquired immunity. There is a diversity of CRISPR-Cas systems based on the set of cas genes and their phylogenetic relationship. There are at least six different types (I through VI) where Type I represents over 50% of all identified systems in both bacteria and archaea. In some embodiments, a Type I, Type II, Type III, Type IV, Type V, or Type VI CRISPR-Cas system is used herein.

Type I systems are divided into seven subtypes including: Type I-A, Type I-B, Type I-C, Type I-D, Type I-E, Type I-F, and Type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and CRISPR array encoding crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA and protospacer-adjacent motifs (PAMs) within the pathogen genome. Base pairing occurs between the crRNA and the target DNA sequence leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand and begins degrading the strand in a 3′-to-5′ direction.

In the Type I-C system, the proteins Cas5, Cas8c, and Cas7 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA and scans DNA to identify PAM sites. In the Type I-C system, the PAM is recognized by the Cas8c protein, which then acts to unwind the DNA duplex. If the sequence 3′ of the PAM matches the crRNA spacer that is bound to effector complex, a conformational change in the complex occurs and Cas3 is recruited to the site. Cas3 then nicks the non-target strand and begins degrading the DNA.

In some embodiments, the CRISPR-Cas system is endogenous to the Escherichia species. In some embodiments, the CRISPR-Cas system is exogenous to the Escherichia species. In some embodiments, the CRISPR-Cas system is a Type I CRISPR-Cas system. In some embodiments, the CRISPR-Cas system is a Type I-A CRISPR-Cas system. In some embodiments, the CRISPR-Cas system is a Type I-B CRISPR-Cas system. In some embodiments, the CRISPR-Cas system is a Type I-C CRISPR-Cas system. In some embodiments, the CRISPR-Cas system is a Type I-D CRISPR-Cas system. In some embodiments, the CRISPR-Cas system is a Type I-E CRISPR-Cas system. In some embodiments, the CRISPR-Cas system is a Type I-F CRISPR-Cas system. In some embodiments, the CRISPR-Cas system is a Type I-U CRISPR-Cas system.

In some embodiments, the CRISPR-Cas system is a Type II CRISPR-Cas system. In some embodiments, the CRISPR-Cas system is a Type III CRISPR-Cas system. In some embodiments, the CRISPR-Cas system is a Type IV CRISPR-Cas system. In some embodiments, the CRISPR-Cas system is a Type VI CRISPR-Cas system.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6, and 3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

### CRISPR Phages

Disclosed herein, in certain embodiments, are bacteriophage compositions comprising CRISPR-Cas systems and methods of use thereof.

Bacteriophages or “phages” represent a group of bacterial viruses and are engineered or sourced from environmental sources. Individual bacteriophage host ranges are usually narrow, meaning, phages are highly specific to one strain or few strains of a bacterial species and this specificity makes them unique in their antibacterial action. Bacteriophages are bacterial viruses that rely on the host's cellular machinery to replicate. Bacteriophages are generally classified as virulent or temperate phages depending on their lifestyle. Virulent bacteriophages, also known as lytic bacteriophages, can only undergo lytic replication. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both they replicative ability and the ability to trigger cell lysis. In some embodiments, the bacteriophages disclosed herein comprise a CRISPR array. In some embodiments, the CRISPR array does not affect the bacteriophages ability to replicate and/or trigger cell lysis. Temperate or lysogenic bacteriophages can undergo lysogeny in which the phage stops replicating and stably resides within the host cell, either integrating into the bacterial genome or being maintained as an extrachromosomal plasmid. Temperate phages can also undergo lytic replication similar to their lytic bacteriophage counterparts. Whether a temperate phage replicates lytically or undergoes lysogeny upon infection depends on a variety of factors including growth conditions and the physiological state of the cell. A bacterial cell that has a lysogenic phage integrated into its genome is referred to as a lysogenic bacterium or lysogen. Exposure to adverse conditions may trigger reactivation of the lysogenic phage, termination of the lysogenic state and resumption of lytic replication by the phage. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication. In some embodiments, the temperate bacteriophages disclosed herein are rendered lytic. The term “lysogeny gene” refers to any gene whose gene product promotes lysogeny of a temperate phage. Lysogeny genes can directly promote, as in the case of integrase proteins that facilitate integration of the bacteriophage into the host genome. Lysogeny genes can also indirectly promote lysogeny as in the case of CI transcriptional regulators which prevent transcription of genes required for lytic replication and thus favor maintenance of lysogeny.

Bacteriophages package and deliver synthetic DNA using three general approaches. Under the first approach, the synthetic DNA is recombined into the bacteriophage genome in a targeted manner, which usually involves a selectable marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Phages have therefore evolved to specifically infect a limited cross-section of bacterial strains. However, some phages have been discovered that inject their genetic material into a wide range of bacteria. The classic example is the P1 phage, which has been shown to inject DNA in a range of gram-negative bacteria.

Disclosed herein, in some embodiments, are bacteriophages comprising a first nucleic acid sequence encoding a first spacer sequence or a crRNA transcribed therefrom, wherein the first spacer sequence is complementary to a target nucleotide sequence from a target gene in a Escherichia species. In some embodiments, the bacteriophage comprises a first nucleic acid sequence encoding a first spacer sequence or a crRNA transcribed therefrom, wherein the first spacer sequence is complementary to a target nucleotide sequence from a target gene in a Escherichia species, provided that the bacteriophage is rendered lytic. In some embodiments, the bacteriophage is a temperate bacteriophage. In some embodiments, the bacteriophage is rendered lytic by removal, replacement, or inactivation of a lysogenic gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the bacteriophage is rendered lytic by the removal of a regulatory element of a lysogeny gene. In some embodiments, the bacteriophage is rendered lytic by the removal of a promoter of a lysogeny gene. In some embodiments, the bacteriophage is rendered lytic by the removal of a functional element of a lysogeny gene. In some embodiments, the lysogenic gene is an activator gene. In some embodiments, the lysogenic gene is cII gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, the bacteriophage is rendered lytic via a second CRISPR array comprising a second spacer directed to a lysogenic gene. In some embodiments, the bacteriophage is rendered lytic by the insertion of one or more lytic genes. In some embodiments, the bacteriophage is rendered lytic by the insertion of one or more genes that contribute to the induction of a lytic cycle. In some embodiments, the bacteriophage is rendered lytic by altering the expression of one or more genes that contribute to the induction of a lytic cycle. In some embodiments, the bacteriophage phenotypically changes from a lysogenic bacteriophage to a lytic bacteriophage. In some embodiments, the phenotypic change is via a self-targeting CRISPR-Cas system to render a bacteriophage lytic since it is incapable of lysogeny. In some embodiments, the self-targeting CRISPR-Cas comprises a self-targeting crRNA from the prophage genome and kills lysogens. In some embodiments, the bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI)). In some embodiments, the bacteriophage that is rendered lytic is prevented from reverting to lysogenic state. In some embodiments, the bacteriophage that is rendered lytic is prevented from reverting back to lysogenic state by way of introducing an additional CRIPSR array. In some embodiments, the bacteriophage does not confer any new properties onto the Escherichia species beyond cellular death cause by lytic activity of the bacteriophage and/or the activity of the CRISPR array. Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a Escherichia species, provided the bacteriophage is rendered lytic. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the Escherichia species. In some embodiments, the gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the target nucleotide sequence is in a non-essential gene. Non-limiting example non-essential genes include ppSa (e.g., SC2), raiA (e.g., SC6), and intergenic conserved repeat (e.g., SC6). In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a Escherichia species. In some embodiments, the spacer sequence is complementary to a target nucleotide sequence of a sequence present in the Escherichia species. In some embodiments, the spacer sequence is complementary to a target nucleotide sequence that comprises all or a part of a promoter sequence of the essential gene. In some embodiments, the first nucleic acid sequence comprises a first CRISPR array comprising at least one repeat sequence. In some embodiments, the at least one repeat sequence is operably linked to the first spacer sequence at either its 5′ end or its 3′ end.

In some embodiments, the bacteriophage or phagemid DNA is from a lysogenic or temperate bacteriophage. In some embodiments, the bacteriophages or phagemids include but are not limited to P1 phage, a M13 phage, a λ phage, a T4 phage, a ϕC2 phage, a ϕCD27 phage, a ϕNM1 phage, Bc431 v3 phage, ϕ10 phage, ϕ25 phage, ϕ151 phage, A511-like phages, B054, 0176-like phages, or Campylobacter phages (such as NCTC 12676 and NCTC 12677). In some embodiments, the bacteriophage includes, but is not limited to p004ke007, p004Ke005, p004K, p00c0, p00ex, p00jc, p00ke, or p5516.

In some embodiments, a plurality of bacteriophages are used together. In some embodiments, the plurality of bacteriophages used together targets the same or different bacteria within a sample or subject.

In some embodiments, bacteriophages of interest are obtained from environmental sources or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, the nucleic acid is inserted into the bacteriophage genome. In some embodiments, the nucleic acid comprises a crArray, a Cas system, or a combination thereof. In some embodiments, the nucleic acid is inserted into the bacteriophage genome at a transcription terminator site at the end of an operon of interest. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle.

Disclosed herein, in certain embodiments, are bacteriophages comprising a complete exogenous CRISPR system. In some embodiments, the CRISPR-Cas system is Type I CRISPR-Cas system, Type II CRISPR-Cas system, Type III CRISPR-Cas system, Type IV CRISPR-Cas system, Type V CRISPR-Cas system, or Type VI CRISPR-Cas system. Disclosed herein, in certain embodiments, are bacteriophages comprising a nucleic acid sequence encoding a Type I CRISPR-Cas system comprising: (a) a CRISPR array; (b) a Cascade polypeptide; and (c) a Cas3 polypeptide. Example Type I component sequences are provided as SEQ ID NOS: 47-54. Disclosed herein, in certain embodiments, are bacteriophages comprising a nucleic acid sequence encoding a Type V CRISPR-Cpf1 (Cas12a) system comprising a CRISPR array associated with a Cpf1 nuclease polypeptide. An example Cpf1 sequence is provided as SEQ ID NO: 46. In some embodiments, a CRISPR system comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 46-54. In some embodiments, a CRISPR system comprises any one of SEQ ID NOS: 46-54. For instance, the CRISPR system comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 46; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 47; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 48; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 49; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 50; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 51; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 52; a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 53; or a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 54; or any combination thereof.

In some embodiments, the bacteriophage is p004ke007. In some embodiments, the bacteriophage is p004Ke005. In some embodiments, the bacteriophage is p004K. In some embodiments, the bacteriophage is p00c0. In some embodiments, the bacteriophage is p00ex. In some embodiments, the bacteriophage is p00jc. In some embodiments, the bacteriophage is p00ke. In some embodiments, the bacteriophage is p5516. In some embodiments, the bacteriophage is p004ke009. In some embodiments, the bacteriophage is p00c0e030. In some embodiments, the bacteriophage is p00exe014. In some embodiments, the bacteriophage comprises a phage listed in Table 1. In some embodiments, the bacteriophage comprises a CRISPR-Cas system.

In some embodiments, a plurality of bacteriophages are used together. In some embodiments, the plurality of bacteriophages used together targets the same or different bacteria within a sample or subject. In some embodiments, a cocktail comprising a plurality of bacteriophages is used together. In some embodiments, the cocktail comprises at least 2, 3, 4, 5, 6, 7, 8, 9, 10, or more phages selected from Table 1. In some embodiments, the cocktail comprises 2 phages selected from Table 1. In some embodiments, the cocktail comprises 3 phages selected from Table 1. In some embodiments, the cocktail comprises 3 phages selected from Table 1. In some embodiments, the cocktail comprises 4 phages selected from Table 1. In some embodiments, the cocktail comprises 5 phages selected from Table 1. In some embodiments, the cocktail comprises 6 phages selected from Table 1. In some embodiments, at least one bacteriophage in the cocktail comprises a CRISPR array (e.g., comprising one or more components of Tables 2-4). In some embodiments, at least 2, 3, 4, 5, 6, 7, 8, 9, 10, or more bacteriophages present in the cocktail comprise a CRISPR array. In some embodiments, at least one bacteriophage in the cocktail comprises a nucleic acid sequence encoding a Cascade polypeptide. In some embodiments, at least 2, 3, 4, 5, 6, 7, 8, 9, 10, or more bacteriophages present in the cocktail comprise a nucleic acid sequence encoding a Cascade polypeptide. In some embodiments, at least one bacteriophage in the cocktail comprises a nucleic acid sequence encoding a Cas3 polypeptide. In some embodiments, at least 2, 3, 4, 5, 6, 7, 8, 9, 10, or more bacteriophages present in the cocktail comprise a nucleic acid sequence encoding a Cas3 polypeptide. In some embodiments, the cocktail comprises p004ke009, p00c0e030, p00exe014, wherein each bacteriophage comprises a Type I CRISPR-Cas system. In some embodiments, the cocktail further comprises p5516. In some embodiments, the cocktail further comprises p00jc. In some embodiments, the cocktail further comprises p00ke.

### CRISPR Array

In some embodiments, the CRISPR array (crArray) comprises a spacer sequence and at least one repeat sequence. In some embodiments, the CRISPR array encodes a processed, mature crRNA. In some embodiments, the mature crRNA is introduced into a phage or a Escherichia species. In some embodiments, an endogenous or exogenous Cas6 processes the CRISPR array into mature crRNA. In some embodiments, an exogenous Cas6 is introduced into the phage. In some embodiments, the phage comprises an exogenous Cas6. In some embodiments, an exogenous Cas6 is introduced into a Escherichia species.

In some embodiments, the CRISPR array comprises a spacer sequence. In some embodiments, the CRISPR array further comprises at least one repeat sequence. In some embodiments, the at least one repeat sequence is operably linked to the spacer sequence at either its end or its 3′ end. In some embodiments, the CRISPR array is of any length and comprises any number of spacer nucleotide sequences alternating with repeat nucleotide sequences necessary to achieve the desired level of killing of a Escherichia species by targeting one or more essential genes. In some embodiments, the CRISPR array comprises, consists essentially of, or consists of 1 to about 100 spacer nucleotide sequences, each linked on its 5′ end and its 3′ end to a repeat nucleotide sequence. In some embodiments, the CRISPR array comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 38. In some embodiments, the CRISPR array comprises SEQ ID NO: 38. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1. In some embodiments, the CRISPR array comprises a promoter from Table 1.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 39. In some embodiments, the CRISPR array comprises SEQ ID NO: 39. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1. In some embodiments, the CRISPR array comprises a promoter from Table 1.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 40. In some embodiments, the CRISPR array comprises SEQ ID NO: 40. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1. In some embodiments, the CRISPR array comprises a promoter from Table 1.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 41. In some embodiments, the CRISPR array comprises SEQ ID NO: 41. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1. In some embodiments, the CRISPR array comprises a promoter from Table 1.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 42. In some embodiments, the CRISPR array comprises SEQ ID NO: 42. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1. In some embodiments, the CRISPR array comprises a promoter from Table 1.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 43. In some embodiments, the CRISPR array comprises SEQ ID NO: 43. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1. In some embodiments, the CRISPR array comprises a promoter from Table 1.

In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 44. In some embodiments, the CRISPR array comprises SEQ ID NO: 44. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a spacer from Table 3 (e.g., 1, 2, or 3 spacers from Table 3). In some embodiments, the CRISPR array comprises a spacer from Table 3. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a repeat from Table 4 (e.g., 1, 2, or 3 spacers from Table 3). In some embodiments, the CRISPR array comprises a repeat from Table 4. In some embodiments, the CRISPR array comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a promoter from Table 1. In some embodiments, the CRISPR array comprises a promoter from Table 1.

### Spacer Sequence

In some embodiments, the spacer sequence is complementary to a target nucleotide sequence in a Escherichia species. In some embodiments, the target nucleotide sequence is a coding region. In some embodiments, the coding region is an essential gene. In some embodiments, the coding region is a nonessential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a Escherichia species. In some embodiments, the spacer sequence is complementary to a target nucleotide sequence of a sequence present in the Escherichia species. In some embodiments, the spacer sequence is complementary to a target nucleotide sequence that comprises all or a part of a promoter sequence of the essential gene. In some embodiments, the spacer sequence comprises one, two, three, four, or five mismatches as compared to the target nucleotide sequence. In some embodiments, the mismatches are contiguous. In some embodiments, the mismatches are noncontiguous. In some embodiments, the spacer sequence has 70% complementarity to a target nucleotide sequence. In some embodiments, the spacer sequence has 80% complementarity to a target nucleotide sequence. In some embodiments, the spacer sequence is 85%, 90%, 95%, 96%, 97%, 98%, 99% or 100% complementarity to a target nucleotide sequence. In some embodiments, the spacer sequence has 100% complementarity to the target nucleotide sequence. In some embodiments, the spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5 ‘ region of the spacer sequence is 100% complementary to a target nucleotide sequence while the 3’ region of the spacer is substantially complementary to the target nucleotide sequence and therefore the overall complementarity of the spacer sequence to the target nucleotide sequence is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target nucleotide sequence, while the remaining nucleotides in the 5′ region of the spacer sequence are substantially complementary (e.g., at least about 70% complementary) to the target nucleotide sequence. In some embodiments, the first 7 to 12 nucleotides of the 3′ end of the spacer sequence is 100% complementary to the target nucleotide sequence, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleotide sequence. In some embodiments, the first 7 to 10 nucleotides in the 3′ end of the spacer sequence is 75%-99% complementary to the target nucleotide sequence, while the remaining nucleotides in the 5′ region of the spacer sequence are at least about 50% to about 99% complementary to the target nucleotide sequence. In some embodiments, the first 7 to 10 nucleotides in the 3′ end of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiments, the first 10 nucleotides (within the seed region) of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiment, the 5′ region of a spacer sequence (e.g., the first 8 nucleotides at the 5′ end, the first 10 nucleotides at the end, the first 15 nucleotides at the 5′ end, the first 20 nucleotides at the 5′ end) have about 75% complementarity or more (75% to about 100% complementarity) to the target nucleotide sequence, while the remainder of the spacer sequence have about 50% or more complementarity to the target nucleotide sequence. In some embodiments, the first 8 nucleotides at the 5′ end of the spacer sequence have 100% complementarity to the target nucleotide sequence or have one or two mutations and therefore is about 88% complementary or about 75% complementary to the target nucleotide sequence, respectively, while the remainder of the spacer nucleotide sequence is at least about 50% or more complementary to the target nucleotide sequence.

In some embodiments, the spacer sequence is about 15 nucleotides to about 150 nucleotides in length. In some embodiments, the spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, the spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about nucleotides, about 10 to about 50 nucleotides, about 10 to about 40, about 10 to about 30, about to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein. In some embodiments, the P. aeruginosa Type I-C Cas system has a spacer length of about to 39 nucleotides, about 31 to about 38 nucleotides, about 32 to about 37 nucleotides, about 33 to about 36 nucleotides, about 34 to about 35 nucleotides, or about 35 nucleotides In some embodiments, the P. aeruginosa Type I-C Cas system has a spacer length of about 34 nucleotides. In some embodiments, the P. aeruginosa Type I-C Cas system has a spacer length of at least about at least about 15, at least about 20, at least about 21, at least about 22, at least about 23, at least about 24, at least about 25, at least about 26, at least about 27, at least about 29, at least about 29, at least about 30, at least about 31, at least about 32, at least about 33, at least about 34, at least about, at least about 35, at least about 36, at least about 37, at least about 38, at least about 39, at least about 20, at least about 41, at least about 42, at least about 43, at least about 44, at least about or more than about 45 nucleotides.

In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO: 12 or 20-37. In some instances, the spacer sequence comprises at least or about 95% homology to SEQ ID NO: 12 or 20-37. In some instances, the spacer sequence comprises at least or about 97% homology to SEQ ID NO: 12 or 20-37. In some instances, the spacer sequence comprises at least or about 99% homology to SEQ ID NO: 12 or 20-37. In some instances, the spacer sequence comprises 100% homology to SEQ ID NO: 12 or 20-37. In some instances, the spacer sequence comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, or more than 34 nucleotides of SEQ ID NO: 12 or 20-37.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

In some embodiments, the identity of two or more spacer sequences of the CRISPR array is the same. In some embodiments, the identity of two or more spacer sequences of the CRISPR array is different. In some embodiments, the identity of two or more spacer sequences of the CRISPR array is different but are complementary to one or more target nucleotide sequences. In some embodiments, the identity of two or more spacer sequences of the CRISPR array is different and are complementary to one or more target nucleotide sequences that are overlapping sequences. In some embodiments, the identity of two or more spacer sequences of the CRISPR array is different and are complementary to one or more target nucleotide sequences that are not overlapping sequences. In some embodiments, the target nucleotide sequence is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to or flanked by a PAM (protospacer adjacent motif).

The PAM sequence is found in the target gene next to the region to which a spacer sequence binds as a result of being complementary to that region and identifies the point at which base pairing with the spacer nucleotide sequence begins. The exact PAM sequence that is required varies between each different CRISPR-Cas system and is identified through established bioinformatics and experimental procedures. Non-limiting examples of PAMs include CCA, CCT, CCG, TTC, AAG, AGG, ATG, GAG, and/or CC. For Type I systems, the PAM is located immediately 5′ to the sequence that matches the spacer, and thus is 3′ to the sequence that base pairs with the spacer nucleotide sequence, and is directly recognized by Cascade. Once a protospacer is recognized, Cascade generally recruits the endonuclease Cas3, which cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a -protospacer adjacent motif recognition domain at the C-terminus of Cas9).

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of the essential gene. In some embodiments, the spacer nucleotide sequence is complementary to a promoter, or a part thereof, of the essential gene. In some embodiments, the target nucleotide sequence comprises all or a part of a nucleotide sequence located on a coding or a non-coding strand of the essential gene. In some embodiments, the target nucleotide sequence comprises all or a part of a nucleotide sequence located on a coding of a transcribed region of the essential gene.

In some embodiments, the essential gene is any gene of an organism that is critical for its survival. However, being essential is highly dependent on the circumstances in which an organism lives. For instance, a gene required to digest starch is only essential if starch is the only source of energy. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the Escherichia species. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the target nucleotide sequence comprises a non-essential gene or a portion thereof. In some embodiments, a non-essential gene is any gene of an organism that is not critical for survival. However, being non-essential is highly dependent on the circumstances in which an organism lives. Non-limiting example non-essential genes include ppSa (e.g., SC2), raiA (e.g., SC6), and intergenic conserved repeat (e.g., SC6).

In some embodiments, non-limiting examples of the target nucleotide sequence of interest includes a target nucleotide sequence encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, the target nucleotide sequence is from a gene involved in cell-division, cell structure, metabolism, motility, pathogenicity, virulence, or antibiotic resistance. In some embodiments, the target nucleotide sequence is from a hypothetical gene whose function is not yet characterized. Thus, for example, these genes are any genes from any bacterium.

The appropriate spacer sequences for a full-construct phage may be identified by locating a search set of representative genomes, searching the genomes with relevant parameters, and determining the quality of a spacer for use in a CRISPR engineered phage.

First, a suitable search set of representative genomes is located and acquired for the organism/species/target of interest. The set of representative genomes may be found in a variety of databases, including without limitations the NCBI genbank or the PATRIC database. NCBI genbank is one of the largest databases available and contains a mixture of reference and submitted genomes for nearly every organism sequenced to date. Specifically, for pathogenic prokaryotes, the PATRIC (Pathosystems Resource Integration Center) database provides an additional comprehensive resource of genomes and provides a focus on clinically relevant strains and genomes relevant to a drug product. Both of the above databases allow for bulk downloading of genomes via FTP (File Transfer Protocol) servers, enabling rapid and programmatic dataset acquisition.

Next, the genomes are searched with relevant parameters to locate suitable spacer sequences. Genomes may be read from start to end, in both the forward and reverse complement orientations, to locate contiguous stretches of DNA that contain a PAM (Protospacer Adjacent Motif) site. The spacer sequence will be the N-length DNA sequence 3′ or 5′ adjacent to the PAM site (depending on the CRISPR system type), where N is specific to the Cas system of interest and is generally known ahead of time. Characterizing the PAM sequence and spacer sequences may be performed during the discovery and initial research of a Cas system. Every observed PAM-adjacent spacer may be saved to a file and/or database for downstream use. The exact PAM sequence that is required varies between each different CRISPR-Cas system and is identified through established bioinformatics and experimental procedures.

Next, the quality of a spacer for use in a CRISPR engineered phage is determined. Each observed spacer may be evaluated to determine how many of the evaluated genomes they are present in. The observed spacers may be evaluated to see how many times they may occur in each given genome. Spacers that occur in more than one location per genome may be advantageous because the Cas system may not be able to recognize the target site if a mutation occurs, and each additional “backup” site increases the likelihood that a suitable, non-mutated target location will be present. The observed spacers may be evaluated to determine whether they occur in functionally annotated regions of the genome. If such information is available, the functional annotations may be further evaluated to determine whether those regions of the genome are “essential” for the survival and function of the organism. By focusing on spacers that occur in all, or nearly all, evaluated genomes of interest (>=99%), the spacer selection may be broadly applicable to many targeted genomes. Provided a large selection pool of conserved spacers exists, preference may be given to spacers that occur in regions of the genome that have known function, with higher preference given if those genomic regions are “essential” for survival and occur more than 1 time per genome.

The spacer sequences for a full construct phage, in some embodiments, are validated. In some embodiments, a first step comprises identifying a plasmid that replicates in the organism, species, or target of interest. In some embodiments, the plasmid has a selectable marker. In some embodiments, the selectable marker is an antibiotic-resistance gene. In some embodiments, an expression cassette includes a nucleotide sequence for a selectable marker. In some embodiments, the selectable marker is adenine deaminase (ada), blasticidin S deaminases (Bsr, BSD), bleomycin-binding protein (Ble), Neomycin phosphotransferase (neo), histidinol dehydrogenase (hisD), glutamine synthetase (GS), dihydrofolate reductase (dhfr), cytosine deaminase (codA), puromycin N-acetyltransferase (Pac), or hygromycin B phosphotransferase (Hph), ampicillin, chloramphenicol, kanamycin, tetracycline, polymyxin B, erythromycin, carbenicillin, streptomycin, spectinomycin, puromycin N-acetyltransferase (Pac), or zeocin (Sh bla). In some embodiments, the selectable marker is a gene involved in thymidylate synthase, thymidine kinase, dihydrofolate reductase, or glutamine synthetase. In some embodiments, the selectable marker is a gene encoding a fluorescent protein.

In some embodiments, a second step comprises inserting the genes encoding the Cas system into the plasmid such that they will be expressed in the organism, species, or target of interest. In some embodiments, a promoter is provided upstream of the Cas system. In some embodiments, the promoter is recognized by the organism, species, or target of interest to drive the expression of the Cas system. Exemplary promoters include, but are not limited to, L-arabinose inducible (araBAD, P BAD) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, GA, GB), Ptms, P43, rplK-rplA, ferredoxin promoter, and/or xylose promoter. In some embodiments, the promoter is a BBa_J23102, BBa_J23104, or BBa_J23109. In some embodiments the promoter is derived from the organism, species, or target bacterium, such as endogenous CRISPR promoter, endogenous Cas operon promoter, p16, plpp, or ptat. In some embodiments, the promoter is a phage promoter, such as the promoter for gp105 or gp245. In some embodiments, a ribosomal binding site (RBS) is provided between the promoter and the Cas system. In some embodiments, the RBS is recognized by the organism, species, or target of interest.

In some embodiments, a third step comprises providing genome-targeting spacers into the plasmid. In some embodiments, the genome-targeting spacers are identified using bioinformatics. In some embodiments, the genome-targeting spacers are provided upstream of the repeat-spacer-repeat. In some embodiments, a promoter is provided. In some embodiments, the promoter is recognized by the organism, species, or target of interest to drive the expression of the crRNA. In some embodiments, the cloning for the third step comprises using an organism or species that is not targeted by the spacer being cloned.

In some embodiments, a fourth step comprises providing a non-target spacer into the plasmid that expresses the Cas system. In some embodiments, the non-target spacer comprises a sequence that is random. In some embodiments, the non-target spacer comprises a sequence that does not comprise targeting sites in the genome of the organism, species, or target of interest. In some embodiments, the non-target spacer sequence is determined using bioinformatics to not comprise targeting sites in the genome of the organism, species, or target of interest. In some embodiments, the non-target spacer sequence is provided upstream of the repeat-spacer-repeat. In some embodiments, a promoter is provided. In some embodiments, the promoter is recognized by the organism, species, or target of interest to drive the expression of the crRNA.

In some embodiments, a fifth step comprises determining an efficacy of each spacer generated. In some embodiments, the killing efficacy is determined. In some embodiments, the efficacy of each spacer at targeting the bacterial genome is determined. In some embodiments, the plasmids comprising the spacer comprises about 0.5-fold, about 1-fold, 5-fold, 10-fold, 20-fold, 40-fold, 60-fold, 80-fold, or up to about 100 fold reduction in transfer rate as compared to a plasmid that comprises the non-targeting spacer.

### Repeat Nucleotide Sequences

In some embodiments, a repeat nucleotide sequence of the CRISPR array comprises a nucleotide sequence of any known repeat nucleotide sequence of a CRISPR-Cas system. In some embodiments, a repeat nucleotide sequence is of a synthetic sequence comprising the secondary structure of a native repeat from a CRISPR-Cas system (e.g., an internal hairpin). In some embodiments, the repeat nucleotide sequences are distinct from one another based on the known repeat nucleotide sequences of a CRISPR-Cas system. In some embodiments, the repeat nucleotide sequences are each composed of distinct secondary structures of a native repeat from a CRISPR-Cas system (e.g., an internal hairpin). In some embodiments, the repeat nucleotide sequences are a combination of distinct repeat nucleotide sequences operable with a CRISPR-Cas system.

In some embodiments, the spacer sequence is linked at its 5′ end to the 3′ end of a repeat sequence. In some embodiments, the spacer sequence is linked at its 5′ end to about 1 to about 8, about 1 to about 10, or about 1 to about 15 nucleotides of the 3′ end of a repeat sequence. In some embodiments, the about 1 to about 8, about 1 to about 10, about 1 to about 15 nucleotides of the repeat sequence are a portion of the 3′ end of a repeat sequence. In some embodiments, the spacer nucleotide sequence is linked at its 3′ end to the 5′ end of a repeat sequence. In some embodiments, the spacer is linked at its 3′ end to about 1 to about 8, about 1 to about 10, or about 1 to about 15 nucleotides of the 5′ end of a repeat sequence. In some embodiments, the about 1 to about 8, about 1 to about 10, about 1 to about 15 nucleotides of the repeat sequence are a portion of the 5′ end of a repeat sequence.

In some embodiments, the spacer nucleotide sequence is linked at its 5′ end to a first repeat sequence and linked at its 3′ end to a second repeat sequence to form a repeat-spacer-repeat sequence. In some embodiments, the spacer sequence is linked at its 5′ end to the 3′ end of a first repeat sequence and is linked at its 3′ end to the 5′ of a second repeat sequence where the spacer sequence and the second repeat sequence are repeated to form a repeat-(spacer-repeat)n sequence such that n is any integer from 1 to 100. In some embodiments, a repeat-(spacer-repeat)n sequence comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 96, 97, 98, 99, 100, or more, spacer nucleotide sequences.

In some embodiments, the repeat sequence is identical to or substantially identical to a repeat sequence from a wild-type CRISPR loci. In some embodiments, the repeat sequence is a repeat sequence found in Table 3. In some embodiments, the repeat sequence is a sequence described herein. In some embodiments, the repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, the repeat sequence comprises, consists essentially of, or consists of at least one nucleotide (e.g., 1, 2, 3, 4, 5, 6, 7, 8, 9, 10, 11, 12, 13, 14, 16, 17, 18, 19, 20, 21, 22, 23, 24, 25, 26, 27, 28, 29, 30, 31, 32, 33, 34, 35, 36, 37, 38, 39, 40, 50, 55, 60, 65, 70, 75, 80, 85, 90, 95, 100, or more nucleotides, or any range therein). In some embodiments, the repeat sequence comprises, consists essentially of, or consists of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides. In some embodiments, the repeat sequence comprises about 20 to 40, 21 to 40, 22 to 23 to 40, 24 to 40, 25 to 40, 26 to 40, 27 to 40, 28 to 40, 29 to 40, 30 to 30, 31 to 40, 32 to 40, 33 to 40, 34 to 40, 35 to 40, 36 to 40, 37 to 40, 38 to 40, 39 to 40, 20 to 39, 20 to 38, 20 to 37, 20 to 36, 20 to 35, 20 to 34, 20 to 33, 20 to 32, 20 to 31, 20 to 30, 20 to 29, 20 to 28, 20 to 26, 20 to 25, to 24, 20 to 23, 20 to 22, or 20 to 21 nucleotides. In some embodiments, the repeat sequence comprises about 20 to 35, 21 to 35, 22 to 35 23 to 35, 24 to 35, 25 to 35, 26 to 35, 27 to 35, 28 to to 35, 30 to 30, 31 to 35, 32 to 35, 33 to 35, 34 to 35, 25 to 40, 25 to 39, 25 to 38, 25 to 37, to 36, 25 to 35, 25 to 34, 25 to 33, 25 to 32, 25 to 31, 25 to 30, 25 to 29, 25 to 28, 25 to 26 nucleotides. In some embodiments, the system is a P. aeruginosa Type I-C Cas system. In some embodiments, the P. aeruginosa Type I-C Cas system has a repeat length of about 25 to 38 nucleotides.

In some embodiments, the repeat sequence comprises at least or about 70%, 80%, 85%, 90%, 91%, 92%, 93%, 94%, 95%, 96%, 97%, 98%, 99%, or 100% sequence identity to any one of SEQ ID NOs: 13-18. In some instances, the repeat sequence comprises at least or about 95% homology to any one of SEQ ID NOS: 13-18. In some instances, the repeat sequence comprises at least or about 97% homology to any one of SEQ ID NOS: 13-18. In some instances, the repeat sequence comprises at least or about 99% homology to any one of SEQ ID NOS: 13-18. In some instances, the repeat sequence comprises 100% homology to any one of SEQ ID NOS: 13-18. In some instances, the repeat sequence comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, or more than 32 nucleotides of any one of SEQ ID NOS: 13-18.

### Type I CRISPR-Cas System

In some embodiments, the Type I CRISPR-Cas system is a Type I-A system, Type I-B system, Type I-C system, Type I-D system, Type I-E system, or Type I-F system. In some embodiments, the Type I CRISPR-Cas system is a Type I-A system. In some embodiments, the Type I CRISPR-Cas system is a Type I-B system. In some embodiments, the Type I CRISPR-Cas system is a Type I-C system. In some embodiments, the Type I CRISPR-Cas system is a Type I-D system. In some embodiments, the Type I CRISPR-Cas system is a Type I-E system. In some embodiments, the Type I CRISPR-Cas system is a Type I-F system. In some embodiments, the Type I CRISPR-Cas system comprises Cascade polypeptides. Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides.

In some embodiments, the Type I Cascade complex comprises: (a) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); (b) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (c) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (d) a nucleotide sequence encoding a Cas1 Od (Csc3) polypeptide, a nucleotide sequence encoding a Csc2 polypeptide, a nucleotide sequence encoding a Csc1 polypeptide, and a nucleotide sequence encoding a Cas6d polypeptide (Type I-D); (e) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6e (CasE) polypeptide (Type I-E); and/or (f) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type I-F).

In some embodiments, the Type I CRISPR-Cas system is exogenous to the Escherichia species.

### Type V CRISPR System

In some embodiments, the CRISPR-Cas system employed in the method disclosed herein is the Type V CRISPR system, using the Cpf1 nuclease (e.g., CRISPR from Prevotella and Francisella1). This monomeric protein with 1200-1500 amino acids length belongs to type V CRISPR system. Cpf1 CRISPR array consists of nine spacer sequences, which are disassociated by 36 nucleotide long repeated sequences. Cpf1 recognizes a 5′-TTTV-3′ PAM in a DNA target, which leads to the base pairing of the spacer-derived segment of the crRNA with the complementary target DNA. Since Cpf1 simultaneously possesses RNAase and DNAase activity, it does not employ tracrRNAs for crRNA biogenesis; instead, the pre-crRNA forms a pseudoknot, that is recognized and cleaved by Cpf1 itself. Cpf1 induces staggered ends (5 or 8 nucleotides 5′ overhang concerning crRNA, length) at the cleaved sites. AM recognition is the first step in Cpf1-mediated gene editing. When PAM is located in the surrounding of a related protospacer, it will set off subsequent hybridization of the crRNA to the target DNA strand and the formation of an R-loop structure. The PAM sequences of Cpf1 family proteins are predominantly T-rich and differ only in the number of thymidines. Also, it was revealed that the nuclease component of Cpf1 recognizes 5′-TTN-3′ PAM on the target strand. PI, REC1, and WED domains altogether participate in the PAM recognition.

### Escherichia

In some embodiments, the bacterium comprises one or more species of Escherichia. In some embodiments, the bacterium comprises one or more strains of Escherichia In some embodiments, the target bacterium is Escherichia coli.

In some embodiments, the E. coli causes an infection or disease. In some embodiments, the infection or disease is acute or chronic. In some embodiments, the infection or disease is localized or systemic. In some embodiments, infection or disease is idiopathic. In some embodiments, the infection or disease is acquired through means including, but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias. In some embodiments, the E. coli causes urinary tract infection. In some embodiments, the E. coli causes and/or exacerbates an inflammatory disease. In some embodiments, the E. coli causes and/or exacerbates an autoimmune disease. In some embodiments, the E. coli causes and/or exacerbates inflammatory bowel disease (IBD). In some embodiments, the E. coli causes inflammatory bowel disease (IBD). In some embodiments, the E. coli causes and/or exacerbates psoriasis. In some embodiments, the E. coli causes and/or exacerbates psoriatic arthritis (PA). In some embodiments, the E. coli causes and/or exacerbates rheumatoid arthritis (RA). In some embodiments, the E. coli causes and/or exacerbates systemic lupus erythematosus (SLE). In some embodiments, the E. coli causes and/or exacerbates multiple sclerosis (MS). In some embodiments, the E. coli causes and/or exacerbates Graves' disease. In some embodiments, the E. coli causes and/or exacerbates Hashimoto's thyroiditis. In some embodiments, the E. coli causes and/or exacerbates Myasthenia gravis. In some embodiments, the E. coli causes and/or exacerbates vasculitis. In some embodiments, the E. coli causes and/or exacerbates cancer. In some embodiments, the E. coli causes and/or exacerbates cancer progression. In some embodiments, the E. coli causes and/or exacerbates cancer metastasis. In some embodiments, the E. coli causes and/or exacerbates resistance to cancer therapy. In some embodiments, the therapy used to address cancer includes, but is not limited to, chemotherapy, immunotherapy, hormone therapy, targeted drug therapy, and/or radiation therapy. In some embodiments, the cancer develops in organs including, but not limited to the, anus, bladder, blood and blood components, bone, bone marrow, brain, breast, cervix uteri, colon and rectum, esophagus, kidney, larynx, lymphatic system, muscle (i.e., soft tissue), oral cavity and pharynx, ovary, pancreas, prostate, skin, small intestine, stomach, testis, thyroid, uterus, and/or vulva. In some embodiments, the E. coli causes and/or exacerbates disorders of the central nervous system (CNS). In some embodiments, the E. coli causes and/or exacerbates attention deficit/hyperactivity disorder (ADHD). In some embodiments, the E. coli causes and/or exacerbates autism. In some embodiments, the E. coli causes and/or exacerbates bipolar disorder. In some embodiments, the E. coli causes and/or exacerbates major depressive disorder. In some embodiments, the E. coli causes and/or exacerbates epilepsy. In some embodiments, the E. coli causes and/or exacerbates neurodegenerative disorders including, but not limited to, Alzheimer's disease, Huntington's disease, and/or Parkinson's disease.

In some embodiments, one or more bacteriophage are administered to a patient with cystic fibrosis or cystic fibrosis-associated bronchiectasis. In some embodiments, a combination of two or more bacteriophage are administered to a patient with cystic fibrosis or cystic fibrosis-associated bronchiectasis. In some embodiments, administration of the bacteriophage to a patient with cystic fibrosis or cystic fibrosis-associated bronchiectasis results in a reduction in bacterial load in the patient. In some embodiments, the reduction in bacterial load results in a clinical improvement in the patient with cystic fibrosis or cystic fibrosis-associated bronchiectasis.

In some embodiments, one or more bacteriophage are administered to a patient with non-cystic fibrosis bronchiectasis. In some embodiments, a combination of two or more bacteriophage are administered to a patient with non-cystic fibrosis bronchiectasis. In some embodiments, administration of the bacteriophage to a patient with non-cystic fibrosis bronchiectasis results in a reduction in bacterial load in the patient. In some embodiments, the reduction in bacterial load results in a clinical improvement in the patient with non-cystic fibrosis bronchiectasis.

### Bacteriophage

In some embodiments, the bacteriophage is an obligate lytic bacteriophage. In some embodiments, the bacteriophage is a temperate bacteriophage with retained lysogeny genes. In some embodiments, the bacteriophage is a temperate bacteriophage with some lysogeny genes removed, replaced, or inactivated. In some embodiments, the bacteriophage is a temperate bacteriophage with a lysogeny gene removed, replaced, or inactivated, thereby rendering the bacteriophage lytic.

In some embodiments, the bacteriophage targets Escherichia spp. In some embodiments, the bacteriophage targets Escherichia coli. In some embodiments, the bacteriophage specifically targets Escherichia spp. over other bacterial species. In some embodiments, the bacteriophage targets Escherichia spp. in the absence of a CRISPR-Cas system.

In some embodiments, the bacteriophage is a Tequatrovirus, a Mosigyvirus, a Phapecoctavirus, a Unique Myoviridae, or a Vectrevirus. In some embodiments, the bacteriophage is a Tequatrovirus. In some embodiments, the bacteriophage is a Mosigyvirus. In some embodiments, the bacteriophage is a Phapecoctavirus. In some embodiments, the bacteriophage is a Unique Myoviridae. In some embodiments, the bacteriophage is a Vectrevirus. In some embodiments, the bacteriophage comprises a CRISPR-Cas3 system.

In some embodiments, the bacteriophage is p004Ke009, p00c0e030, p00exe014, p004ke007, p004Ke005, p004K, p00c0, p00ex, p00jc, p00ke, or p5516, which target Escherichia ssp. In some embodiments, the bacteriophages include, but are not limited to, p004ke007, p004Ke005, p004K, p00c0, p00ex, p00jc, p00ke, or p5516. In some embodiments, “a bacteriophage” is inclusive of one or more bacteriophage, where a first bacteriophage and a second bacteriophage are different or the same. For instance, a bacteriophage comprises p004Ke009, p00c0e030, and p00exe014.

In some embodiments, the bacteriophage is p004K, or a mutant thereof which retains the ability to target Escherichia spp. In some embodiments, the bacteriophage comprises at least 70%, 75%, 80%, 85%, 90% 95%, or 100% sequence identity with p004K. In some embodiments, the bacteriophage is a p004K bacteriophage comprising a CRISPR system. In some embodiments, the bacteriophage comprises at least 70%, 75%, 80%, 85%, 90% 95%, or 100% sequence identity with p004ke005. In some embodiments, the bacteriophage comprises at least 70%, 75%, 80%, 85%, 90% 95%, or 100% sequence identity with p004ke007. In some embodiments, the bacteriophage comprises at least 70%, 75%, 80%, 85%, 90% 95%, or 100% sequence identity with p004ke009.

In some embodiments, the bacteriophage is p00c0, or a mutant thereof which retains the ability to target Escherichia spp. In some embodiments, the bacteriophage comprises at least 70%, 75%, 80%, 85%, 90% 95%, or 100% sequence identity with p00c0. In some embodiments, the bacteriophage is a p00c0 bacteriophage comprising a CRISPR system. In some embodiments, the bacteriophage comprises at least 70%, 75%, 80%, 85%, 90% 95%, or 100% sequence identity with p00c0e030.

In some embodiments, the bacteriophage is p00ex, or a mutant thereof which retains the ability to target Escherichia spp. In some embodiments, the bacteriophage comprises at least 70%, 75%, 80%, 85%, 90% 95%, or 100% sequence identity with p00ex. In some embodiments, the bacteriophage is a p00ex bacteriophage comprising a CRISPR system. In some embodiments, the bacteriophage comprises at least 70%, 75%, 80%, 85%, 90% 95%, or 100% sequence identity with p00exe014.

In some embodiments, the bacteriophage is p00jc, or a mutant thereof which retains the ability to target Escherichia spp. In some embodiments, the bacteriophage comprises at least 70%, 75%, 80%, 85%, 90% 95%, or 100% sequence identity with p00jc. In some embodiments, the bacteriophage is a p00jc bacteriophage comprising a CRISPR system.

In some embodiments, the bacteriophage is p00ke, or a mutant thereof which retains the ability to target Escherichia spp. In some embodiments, the bacteriophage comprises at least 70%, 75%, 80%, 85%, 90% 95%, or 100% sequence identity with p00ke. In some embodiments, the bacteriophage is a p00ke bacteriophage comprising a CRISPR system.

In some embodiments, the bacteriophage is p5516, or a mutant thereof which retains the ability to target Escherichia spp. In some embodiments, the bacteriophage comprises at least 70%, 75%, 80%, 85%, 90% 95%, or 100% sequence identity with p5516. In some embodiments, the bacteriophage is a p5516 bacteriophage comprising a CRISPR system.

In some embodiments, the bacteriophage comprises a phage listed in Table 1, or a mutant thereof, which retains the ability to target Escherichia spp.

Also disclosed herein is a cocktail comprising two or more bacteriophage. In some embodiments, the two or more bacteriophage are selected from the lineage consisting of a Tequatrovirus, a Mosigyvirus, a Phapecoctavirus, a Unique Myoviridae, or a Vectrevirus. In some embodiments, the cocktail comprises at least six bacteriophage, wherein the bacteriophage comprise a Tequatrovirus, a Mosigyvirus, a Phapecoctavirus, a Unique Myoviridae, and a Vectrevirus. In some embodiments, at least one bacteriophage of the cocktail comprises a CRISPR-Cas system. In some embodiments, at least two bacteriophages of the cocktail comprise a CRISPR-Cas system. In some embodiments, at least three bacteriophage of the cocktail comprise a CRISPR-Cas system. In some embodiments, at least four bacteriophage of the cocktail comprise a CRISPR-Cas system. In some embodiments, at least one bacteriophage of the cocktail does not comprise a CRISPR-Cas system. In some embodiments, at least two bacteriophages of the cocktail do not comprise a CRISPR-Cas system.

In some embodiments, the cocktail comprises at least two bacteriophage, wherein the bacteriophage comprise p004K, p00c0, p00ex, p00jc, p00ke, or p5516. In some embodiments, the cocktail comprises a first bacteriophage comprising at least 70%, 75%, 80%, 85%, 90%, 95%, or 100% sequence identity with p004ke009. In some embodiments, the cocktail comprises a second bacteriophage comprising at least 70%, 75%, 80%, 85%, 90%, 95%, or 100% sequence identity with p00c0e030. In some embodiments, the cocktail comprises a third bacteriophage comprising at least 70%, 75%, 80%, 85%, 90%, 95%, or 100% sequence identity with p00exe014. In some embodiments, the cocktail comprises a fourth bacteriophage comprising at least 70%, 75%, 80%, 85%, 90%, 95%, or 100% sequence identity with p00exe014. In some embodiments, the cocktail comprises a fifth bacteriophage comprising at least 70%, 75%, 80%, 85%, 90%, 95%, or 100% sequence identity with p00jc. In some embodiments, the cocktail comprises a fifth bacteriophage comprising at least 70%, 75%, 80%, 85%, 90%, 95%, or 100% sequence identity with p00jc, wherein the bacteriophage comprises a CRISPR-Cas system. In some embodiments, the cocktail comprises a fifth bacteriophage comprising at least 70%, 75%, 80%, 85%, 90%, 95%, or 100% sequence identity with p00ke. In some embodiments, the cocktail comprises a fifth bacteriophage comprising at least 70%, 75%, 80%, 85%, 90%, 95%, or 100% sequence identity with p00ke, wherein the bacteriophage comprises a CRISPR-Cas system. In some embodiments, the cocktail comprises a sixth bacteriophage comprising at least 70%, 75%, 80%, 85%, 90%, 95%, or 100% sequence identity with p5516.

In some embodiments, the first bacteriophage comprises at least 70%, 75%, 80%, 85%, 90% 95%, or 100% sequence identity with p004K. In some embodiments, the cocktail comprises a second bacteriophage, wherein the second bacteriophage comprises at least 70%, 75%, 80%, 85%, 90% 95%, or 100% with p00c0, p00ex, p00jc, p00ke, or p5516. In some embodiments, the cocktail comprises a third bacteriophage, wherein the third bacteriophage comprises at least 70%, 75%, 80%, 85%, 90% 95%, or 100% with p00c0, p00ex, p00jc, p00ke, or p5516. In some embodiments, the cocktail comprises a fourth bacteriophage, wherein the fourth bacteriophage comprises at least 70%, 75%, 80%, 85%, 90% 95%, or 100% with p00c0, p00ex, p00jc, p00ke, or p5516. In some embodiments, the cocktail comprises a fifth bacteriophage, wherein the fifth bacteriophage comprises at least 70%, 75%, 80%, 85%, 90% 95%, or 100% with p00c0, p00ex, p00jc, p00ke, or p5516. In some embodiments, the cocktail comprises a sixth bacteriophage, wherein the sixth bacteriophage comprises at least 70%, 75%, 80%, 85%, 90% 95%, or 100% with p00c0, p00ex, p00jc, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00c0. In some embodiments, the cocktail comprises a second bacteriophage, wherein the second bacteriophage comprises at least 70%, 75%, 80%, 85%, 90% 95%, or 100% with p004K, p00ex, p00jc, p00ke, or p5516. In some embodiments, the cocktail comprises a third bacteriophage, wherein the third bacteriophage comprises at least 70%, 75%, 80%, 85%, 90% 95%, or 100% with p004K, p00ex, p00jc, p00ke, or p5516. In some embodiments, the cocktail comprises a fourth bacteriophage, wherein the fourth bacteriophage comprises at least 70%, 75%, 80%, 85%, 90% 95%, or 100% with p004K, p00ex, p00jc, p00ke, or p5516. In some embodiments, the cocktail comprises a fifth bacteriophage, wherein the fifth bacteriophage comprises at least 70%, 75%, 80%, 85%, 90% 95%, or 100% with p004K, p00ex, p00jc, p00ke, or p5516. In some embodiments, the cocktail comprises a sixth bacteriophage, wherein the sixth bacteriophage comprises at least 70%, 75%, 80%, 85%, 90% 95%, or 100% with p004K, p00ex, p00jc, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00ex. In some embodiments, the cocktail comprises a second bacteriophage, wherein the second bacteriophage comprises at least 70%, 75%, 80%, 85%, 90% 95%, or 100% with p00c0, p004K, p00jc, p00ke, or p5516. In some embodiments, the cocktail comprises a third bacteriophage, wherein the third bacteriophage comprises at least 70%, 75%, 80%, 85%, 90% 95%, or 100% with p00c0, p004K, p00jc, p00ke, or p5516. In some embodiments, the cocktail comprises a fourth bacteriophage, wherein the fourth bacteriophage comprises at least 70%, 75%, 80%, 85%, 90% 95%, or 100% with p00c0, p004K, p00jc, p00ke, or p5516. In some embodiments, the cocktail comprises a fifth bacteriophage, wherein the fifth bacteriophage comprises at least 70%, 75%, 80%, 85%, 90% 95%, or 100% with p00c0, p004K, p00jc, p00ke, or p5516. In some embodiments, the cocktail comprises a sixth bacteriophage, wherein the sixth bacteriophage comprises at least 70%, 75%, 80%, 85%, 90% 95%, or 100% with p00c0, p004K, p00jc, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00jc. In some embodiments, the cocktail comprises a second bacteriophage, wherein the second bacteriophage comprises at least 70%, 75%, 80%, 85%, 90% 95%, or 100% with p00c0, p00ex, p004K, p00ke, or p5516. In some embodiments, the cocktail comprises a third bacteriophage, wherein the third bacteriophage comprises at least 70%, 75%, 80%, 85%, 90% 95%, or 100% with p00c0, p00ex, p004K, p00ke, or p5516. In some embodiments, the cocktail comprises a fourth bacteriophage, wherein the fourth bacteriophage comprises at least 70%, 75%, 80%, 85%, 90% 95%, or 100% with p00c0, p00ex, p004K, p00ke, or p5516. In some embodiments, the cocktail comprises a fifth bacteriophage, wherein the fifth bacteriophage comprises at least 70%, 75%, 80%, 85%, 90% 95%, or 100% with p00c0, p00ex, p004K, p00ke, or p5516. In some embodiments, the cocktail comprises a sixth bacteriophage, wherein the sixth bacteriophage comprises at least 70%, 75%, 80%, 85%, 90% 95%, or 100% with p00c0, p00ex, p004K, p00ke, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p00ke. In some embodiments, the cocktail comprises a second bacteriophage, wherein the second bacteriophage comprises at least 70%, 75%, 80%, 85%, 90% 95%, or 100% with p00c0, p00ex, p00jc, p004K, or p5516. In some embodiments, the cocktail comprises a third bacteriophage, wherein the third bacteriophage comprises at least 70%, 75%, 80%, 85%, 90% 95%, or 100% with p00c0, p00ex, p00jc, p004K, or p5516. In some embodiments, the cocktail comprises a fourth bacteriophage, wherein the fourth bacteriophage comprises at least 70%, 75%, 80%, 85%, 90% 95%, or 100% with p00c0, p00ex, p00jc, p004K, or p5516. In some embodiments, the cocktail comprises a fifth bacteriophage, wherein the fifth bacteriophage comprises at least 70%, 75%, 80%, 85%, 90% 95%, or 100% with p00c0, p00ex, p00jc, p004K, or p5516. In some embodiments, the cocktail comprises a sixth bacteriophage, wherein the sixth bacteriophage comprises at least 70%, 75%, 80%, 85%, 90% 95%, or 100% with p00c0, p00ex, p00jc, p004K, or p5516. In some embodiments, at least one, two, three, four, or five bacteriophage comprise a CRISPR-Cas system. In some embodiments, at least one, two, or three bacteriophage do not comprise a CRISPR Cas system.

In some embodiments, the first bacteriophage comprises at least 70%, 75%, 80%, 85%, 90% 95%, or 100% sequence identity with p5516. In some embodiments, the cocktail comprises a second bacteriophage, wherein the second bacteriophage comprises at least 70%, 75%, 80%, 85%, 90% 95%, or 100% with p00c0, p00ex, p00jc, p00ke, or p004K. In some embodiments, the cocktail comprises a third bacteriophage, wherein the third bacteriophage comprises at least 70%, 75%, 80%, 85%, 90% 95%, or 100% with p00c0, p00ex, p00jc, p00ke, or p004K. In some embodiments, the cocktail comprises a fourth bacteriophage, wherein the fourth bacteriophage comprises at least 70%, 75%, 80%, 85%, 90% 95%, or 100% with p00c0, p00ex, p00jc, p00ke, or p004K. In some embodiments, the cocktail comprises a fifth bacteriophage, wherein the fifth bacteriophage comprises at least 70%, 75%, 80%, 85%, 90% 95%, or 100% with p00c0, p00ex, p00jc, p00ke, or p004K. In some embodiments, the cocktail comprises a sixth bacteriophage, wherein the sixth bacteriophage comprises at least 70%, 75%, 80%, 85%, 90% 95%, or 100% with p00c0, p00ex, p00jc, p00ke, or p004K. In some embodiments, at least one, two, three, four, or five bacteriophage comprise a CRISPR-Cas system. In some embodiments, at least one, two, or three bacteriophage do not comprise a CRISPR Cas system.

In some embodiments, bacteriophages of interest are obtained from environmental sources or from commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

### Insertion Sites

In some embodiments, the insertion of the nucleic acid sequence into a bacteriophage preserves the lytic activity of the bacteriophage. In some embodiments, the nucleic acid sequence is inserted into the bacteriophage genome. In some embodiments, the nucleic acid sequence is inserted into the bacteriophage genome at a transcription terminator site at the end of an operon of interest. In some embodiments, the nucleic acid sequence is inserted into the bacteriophage genome as a replacement for one or more removed non-essential genes. In some embodiments, the nucleic acid sequence is inserted into the bacteriophage genome as a replacement for one or more removed lysogenic genes. In some embodiments, the replacement of non-essential and/or lysogenic genes with the nucleic acid sequence does not affect the lytic activity of the bacteriophage. In some embodiments, the replacement of non-essential and/or lysogenic genes with the nucleic acid sequence preserves the lytic activity of the bacteriophage. In some embodiments, the replacement of non-essential and/or lysogenic genes with the nucleic acid sequence enhances the lytic activity of the bacteriophage. In some embodiments, the replacement of non-essential and/or lysogenic genes with the nucleic acid sequence renders a lysogenic bacteriophage lytic.

In some embodiments, the nucleic acid sequence is introduced into the bacteriophage genome at a first location while one or more non-essential and/or lysogenic genes are separately removed and/or inactivated from the bacteriophage genome at a separate location. In some embodiments, the nucleic acid sequence is introduced into the bacteriophage at a first location while one or more non-essential and/or lysogenic genes are separately removed and/or inactivated from the bacteriophage genome at multiple separate locations. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way the self-targeting activity of the first introduced CRISPR array. In some embodiments, a temperate bacteriophage that is rendered lytic is prevented from reverting back to lysogenic state by way of introducing an additional CRISPR array. In some embodiments, the bacteriophage does not confer any new properties onto the target bacterium beyond cellular death cause by lytic activity of the bacteriophage and/or the activity of the first or second CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

### Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle.

### Transcriptional Activators

In some embodiments, the nucleic acid sequence further comprises a transcriptional activator. In some embodiments, the transcriptional activator encoded regulates the expression of genes of interest within the Escherichia species. In some embodiments, the transcriptional activator activates the expression of genes of interest within the Escherichia species whether exogenous or endogenous. In some embodiments, the transcriptional activator activates the expression genes of interest within the Escherichia species by disrupting the activity of one or more inhibitory elements within the Escherichia species. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending. In some bacteria, such as E. coli, the regulation of the CRISPR-Cas3 operon is regulated by H-NS.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur. As such, CodY acts as a stringent global transcriptional repressor.

In some embodiments, the transcriptional activator is a LeuO polypeptide, homolog or functional fragment thereof, a leuO coding sequence, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid sequence comprising a CRISPR array. In some embodiments, the increase in the expression of a CRISPR-Cas system due to the disruption of an inhibitory element by the expression of LeuO causes an increase in the CRISPR-Cas processing of a nucleic acid sequence comprising a CRISPR array so as to increase the level of lethality of the CRISPR array against a bacterium. In some embodiments, transcriptional activator causes increase activity of a bacteriophage and/or the CRISPR-Cas system.

### Regulatory Elements

In some embodiments, the nucleic acid sequences are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked the CRISPR array. Any promoter useful with this disclosure is used and includes, for example, promoters functional with the organism of interest as well as constitutive, inducible, developmental regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, P BAD) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase σ factor recognition sites, σA, σB), Ptms, P43, rplK-rplA, ferredoxin promoter, and/or xylose promoter. In some embodiments, the promoter is a BBa_J23102 promoter. In some embodiments, the promoter works in a broad range of bacteria, such as BBa_J23104, BBa_J23109. In some embodiments the promoter is derived from the target bacterium, such as endogenous CRISPR promoter, endogenous Cas operon promoter, p16, plpp, or ptat. In some embodiments, the promoter is a phage promoter, such as the promoter for gp105 or gp245.

In some embodiments, the promoter comprises at least or about 70%, 80%, 85%, 90%, 91%, 92%, 93%, 94%, 95%, 96%, 97%, 98%, 99%, or 100% sequence identity to any one of SEQ ID NOs: 1-11 or 19. In some instances, the promoter comprises at least or about 95% homology to any one of SEQ ID NOS: 1-11 or 19. In some instances, the promoter comprises at least or about 97% homology to any one of SEQ ID NOS: 1-11 or 19. In some instances, the promoter comprises at least or about 99% homology to any one of SEQ ID NOS: 1-11 or 19. In some instances, the promoter comprises 100% homology to any one of SEQ ID NOS: 1-11 or 19. In some instances, the promoter comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides of any one of SEQ ID NOS: 1-11 or 19. In some instances, the promoter comprises at least a portion having at least or about 50, 55, 60, 65, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 nucleotides of any one of SEQ ID NOS: 1-11 or 19.

In some embodiments, inducible promoters are used. In some embodiments, chemical-regulated promoters are used to modulate the expression of a gene in an organism through the application of an exogenous chemical regulator. The use of chemically regulated promoters enables RNAs and/or the polypeptides encoded by the nucleic acid sequence to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

### Expression Cassette

In some embodiments, the nucleic acid sequence is an expression cassette or in an expression cassette. In some embodiments, the expression cassettes are designed to express the nucleic acid sequence disclosed herein. In some embodiments, the nucleic acid sequence is an expression cassette encoding components of a CRISPR-Cas system. In some embodiments, the nucleic acid sequence is an expression cassette encoding components of a Type I CRISPR-Cas system. In some embodiments, the nucleic acid sequence is an expression cassette encoding an operable CRISPR-Cas system. In some embodiments, the nucleic acid sequence is an expression cassette encoding the operable components of a Type I CRISPR-Cas system, including Cascade and Cas3. In some embodiments, the nucleic acid sequence is an expression cassette encoding the operable components of a Type I CRISPR-Cas system, including a crRNA, Cascade and Cas3.

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

### Vectors

In addition to expression cassettes, the nucleic acid sequences disclosed herein (e.g. nucleic acid sequence comprising a CRISPR array) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid vector, a fosmid vector, a bacteriophage, an artificial chromosome, or an Agrobacterium binary vector in double or single stranded linear or circular form which may or may not be self-transmissible or mobilizable. A vector transforms prokaryotic or eukaryotic host either by integration into the cellular genome or exist extrachromosomally (e.g. autonomous replicating plasmid with an origin of replication). Additionally, included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### Codon Optimization

In some embodiments, the nucleic acid sequence is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

### Transformation

In some embodiments, the nucleic acid sequence, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Antimicrobial Agents and Peptides

In some embodiments, a bacteriophage disclosed herein is further genetically modified to express an antibacterial peptide, a functional fragment of an antibacterial peptide or a lytic gene. In some embodiments, a bacteriophage disclosed herein express at least one antimicrobial agent or peptide disclosed herein. In some embodiments, a bacteriophage disclosed herein comprises a nucleic acid sequence that encodes an enzybiotic where the protein product of the nucleic acid sequence targets phage resistant bacteria. In some embodiments, the bacteriophage comprises nucleic acids which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase, colanic acid, depolymerazing alginase, DNase I, or combinations thereof. In some embodiments, a bacteriophage disclosed herein secretes an enzyme disclosed herein.

In some embodiments, an antimicrobial agent or peptide is expressed and/or secreted by a bacteriophage disclosed herein. In some embodiments, a bacteriophage disclosed herein secretes and expresses an antibiotic such as ampicillin, penicillin, penicillin derivatives, cephalosporins, monobactams, carbapenems, ofloxacin, ciprofloxacin, levofloxacin, gatifloxacin, norfloxacin, lomefloxacin, trovafloxacin, moxifloxacin, sparfloxacin, gemifloxacin, pazufloxacin or any antibiotic disclosed herein. In some embodiments, a bacteriophage disclosed herein comprises a nucleic acid sequence encoding an antibacterial peptide, expresses an antibacterial peptide, or secretes a peptide that aids or enhances killing of a E. coli. In some embodiments, a bacteriophage disclosed herein comprises a nucleic acid sequence encoding a peptide, a nucleic acid sequence encoding an antibacterial peptide, expresses an antibacterial peptide, or secretes a peptide that aids or enhances the activity of the first and/or the second Type I CRISPR-Cas system.

### Methods of Use

Disclosed herein, in certain embodiments, are methods of killing a E. coli comprising introducing into the E. coli any of the bacteriophages disclosed herein.

Further disclosed herein, in certain embodiments, are methods of modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells any of the bacteriophages disclosed herein.

Also disclosed herein, in certain embodiments, are methods of treating a disease in an individual in need thereof, the method comprising administering to the individual any of the bacteriophages disclosed herein.

In some embodiments, the E. coli is killed solely by lytic activity of the bacteriophage. In some embodiments, the E. coli is killed solely by activity of the CRISPR-Cas system. In some embodiments, the E. coli is killed by the processing of the CRISPR array by a CRISPR-Cas system to produce a processed crRNA capable of directing CRISPR-Cas based endonuclease activity and/or cleavage at the target nucleotide sequence in the target gene of the bacterium.

In some embodiments, the E. coli is killed by lytic activity of the bacteriophage in combination with activity of the Type I CRISPR-Cas system. In some embodiments, the E. coli is killed by the activity of the Type I CRISPR-Cas system, independently of the lytic activity of the bacteriophage. In some embodiments, the activity of the Type I CRISPR-Cas system supplements or enhances the lytic activity of the bacteriophage. In some embodiments, the activity of the Type I CRISPR-Cas system and the lytic activity of the bacteriophage are additive.

In some embodiments, the lytic activity of the bacteriophage and the activity of the Type I CRISPR-Cas system is synergistic. In some embodiments, a synergistic activity is defined as an activity resulting in a greater level of phage kill than the additive combination of the lytic activity of the bacteriophage and the Type I CRISPR-Cas system. In some embodiments, the lytic activity of the bacteriophage is modulated by a concentration of the bacteriophage. In some embodiments, the activity of the Type I CRISPR-Cas system is modulated by a concentration of the bacteriophage.

In some embodiments, the synergistic killing of the bacterium is modulated to favor killing by the lytic activity of the bacteriophage over the activity of the first CRISPR-Cas system by increasing the concentration of bacteriophage administered to the bacterium. In some embodiments, the synergistic killing of the bacterium is modulated to disfavor killing by the lytic activity of the bacteriophage over the activity of the CRISPR-Cas system by decreasing the concentration of bacteriophage administered to the bacterium. In some embodiments, at low concentrations, lytic replication allows for amplification and killing of the target bacteria. In some embodiments, at high concentrations, amplification of a phage is not required. In some embodiments, the synergistic killing of the bacterium is modulated to favor killing by the activity of the CRISPR-Cas system over the lytic activity of the bacteriophage by altering the number, the length, the composition, the identity, or any combination thereof, of the spacers so as to increase the lethality of the CRISPR array. In some embodiments, the synergistic killing of the bacterium is modulated to disfavor killing by the activity of the CRISPR-Cas system over the lytic activity of the bacteriophage by altering the number, the length, the composition, the identity, or any combination thereof, of the spacers so as to decrease the lethality of the CRISPR array.

### Administration Routes and Dosage

Dose and duration of the administration of a composition disclosed herein will depend on a variety of factors, including the subject's age, subject's weight, and tolerance of the phage. In some embodiments, a bacteriophage disclosed herein is administered to patients intra-arterially, intravenously, intraurethrally, intramuscularly, orally, subcutaneously, by inhalation, or any combination thereof. In some embodiments, a bacteriophage disclosed herein is administered to patients by oral administration. In some embodiments, a bacteriophage disclosed herein is administered to patients by topical, cutaneous, transdermal, transmucosal, implantation, sublingual, buccal, rectal, vaginal, ocular, otic, or nasal administration. In some embodiments, a bacteriophage disclosed herein is administered to patients by any combination of the aforementioned routes of administration.

In some embodiments, a dose of phage between 10³ and 10²⁰ PFU is given. In some embodiments, a dose of phage between 10³ and 10¹⁰ PFU is given. In some embodiments, a dose of phage between 10⁶ and 10²⁰ PFU is given. In some embodiments, a dose of phage between 10⁶ and 10¹⁰ PFU is given. For example, in some embodiments, the bacteriophage is present in a composition in an amount between 10³ and 10¹⁰ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU or more. In some embodiments, the bacteriophage is present in a composition in an amount of less than 10¹ PFU. In some embodiments, the bacteriophage is present in a composition in an amount between 10¹ and 10⁸, 10⁴ and 10⁹, 10⁵ and 10¹⁰, or 10⁷ and 10¹¹ PFU. In some embodiments, a composition comprising two or more bacteriophage is administered to a subject, wherein each bacteriophage is administered in an amount about 103, 104, 105, 106, 107, 108, 109, 1010, 1011, 1012, 1013, 1014, 1015, 1016, 1017, 1018, 1019, 1020, 1021, 1022, 1023, 1024 PFU or more. In some embodiments, a composition comprising two or more bacteriophage is administered to a subject, wherein each bacteriophage is administered in an amount of less than 101 PFU. In some embodiments, a composition comprising two or more bacteriophage is administered to a subject, wherein each bacteriophage is administered in an amount between 101 and 108, 104 and 109, 105 and 1010, or 107 and 1011 PFU.

In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some embodiments, a bacteriophage or a mixture is administered to a subject in need thereof every 2, 4, 6, 8, 10, 12, 14, 18, 20, 22, or 24 hours.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Bacterial Infections

Disclosed herein, in certain embodiments, are methods of treating bacterial infections. In some embodiments, the bacteriophages disclosed herein treat or prevent diseases or conditions mediated or caused by bacteria as disclosed herein in a human or animal subject. In some embodiments, the bacteriophages disclosed herein treat or prevent diseases or conditions caused or exacerbated by bacteria as disclosed herein in a human or animal subject. Such bacteria are typically in contact with tissue of the subject including: gut, oral cavity, lung, armpit, ocular, vaginal, anal, ear, nose or throat tissue. In some embodiments, a bacterial infection is treated by modulating the activity of the bacteria and/or by directly killing of the bacteria.

In some embodiments, the bacterium is Escherichia spp. In some embodiments, the bacterium is Escherichia coli

In some embodiments, one or more Escherichia species present in a bacterial population are pathogenic. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are Shiga-toxin producing. In some embodiments, the pathogenic bacterium is Shiga-toxin producing E. coli (STEC). In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the bacteriophages disclosed herein are used to treat an infection, a disease, or a condition, in the gastrointestinal tract of a subject. In some embodiments, the bacteriophages are used to modulate and/or kill target bacteria within the microbiome or gut flora of a subject. In some embodiments, the bacteriophages are used to selectively modulate and/or kill one or more target bacteria from a plurality of bacteria within the microbiome or gut flora of a subject. In some embodiments, the bacteriophages are used to selectively modulate and/or kill one or more target enteropathogenic bacteria from a plurality of bacteria within the microbiome or gut flora of a subject. In some embodiments, the target enteropathogenic bacterium is enteropathogenic E. coli (EPEC). In some embodiments, the bacteriophages are used to selectively modulate and/or kill one or more target diarrheagenic bacteria from a plurality of bacteria within the microbiome or gut flora of a subject. In some embodiments, the target diarrheagenic bacterium is diarrheagenic E. coli (DEC). In some embodiments, the bacteriophages are used to selectively modulate and/or kill one or more target Shiga-toxin producing bacteria from a plurality of bacteria within the microbiome or gut flora of a subject. In some embodiments, the target Shiga-toxin producing bacterium is Shiga-toxin producing E. coli (STEC).

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. The urinary tract flora includes, but is not limited, to Staphylococcus epidermidis, Enterococcus faecalis, and some alpha-hemolytic Streptococci. In some embodiments, the bacteriophages are used to selectively modulate and/or kill one or more target uropathogenic bacteria from a plurality of bacteria within the urinary tract flora of a subject. In some embodiments, the target bacterium is uropathogenic E. coli (UPEC).

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, on a mucosal membrane of a subject. In some embodiments, the bacteriophages are used to modulate and/or kill target bacteria on the mucosal membrane of a subject.

In some embodiments, the pathogenic bacteria are antibiotic resistant. In some embodiments, the pathogenic bacteria are extended-spectrum beta-lactamase (ESBL) producing. In one embodiment, the pathogenic bacterium is carbapenem-resistant E. coli.

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, the bacterium is includes Escherichia spp. In some embodiments, the bacterium is Escherichia coli.

In some embodiments, the bacteriophage treats acne and other related skin infections.

In some embodiments, the Escherichia species is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, the bacteria strain is Escherichia coli.

In some embodiments, the bacterium is E. coli. In some embodiments, the methods and compositions disclosed herein are for use in veterinary and medical applications as well as research applications.

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteroidetes, or Firmicutes.

In some embodiments, the bacteriophages as disclosed herein are used to modulate or kill target bacteria within the microbiome of a subject. In some embodiments, the bacteriophages are used to modulate and/or kill target bacteria within the microbiome by the CRISPR-Cas system, lytic activity, or a combination thereof. In some embodiments, the bacteriophages are used to modulate and/or kill target bacteria within the microbiome of a subject. In some embodiments, the bacteriophages are used to selectively modulate and/or kill one or more target bacteria from a plurality of bacteria within the microbiome of a subject. In some embodiments, the target bacterium is E. coli. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary bacteria associated with diseases and conditions of gastrointestinal tract and are being modulated or killed by the bacteriophages include strains, sub-strains, and enterotypes of E. coli.

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary list of the bacteria associated with diseases and conditions of gastrointestinal tract and are being modulated or killed by the bacteriophages include strains, sub-strains, and enterotypes of enterobacteriaceae, pasteurellaceae, fusobacteriaceae, neisseriaceae, veillonellaceae, gemellaceae, bacteriodales, clostridiales, erysipelotrichaceae, bifidobacteriaceae bacteroides, Faecalibacterium, Roseburia, Blautia, Ruminococcus, Coprococcus, Streptococcus, Dorea, Blautia, Ruminococcus, Lactobacillus, Enterococcus, Streptococcus, Escherichia coli, Fusobacterium nucleatum, Haemophilus parainfluenzae (pasteurellaceae), Veillonella parvula, Eikenella corrodens (neisseriaceae), Gemella moribillum, Bacteroides vulgatus, Bacteroides caccae, Bifidobacterium bifidum, Bifidobacterium longum, Bifidobacterium adolescentis, Bifidobacterium Dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, Coprococcus comes, Actinomyces, Lactococcus, Roseburia, Streptococcus, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Enterococcus, Pediococcus, Citrobacter, Odoribacter, Enterobacteria, Fusobacterium, and Proteus.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic or a third agent. In some embodiment, microbiome related disease or disorder is treated by a bacteriophage disclosed herein.

### Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiment a solution described herein comprises between 10¹-10²⁰ plaque forming units (PFU)/ml. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

### Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiment, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or be transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

### Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products. For example, freshly-cut produce frequently arrive at the processing plant contaminated with pathogenic bacteria. This has led to outbreaks of food-borne illness traceable to produce. In some embodiments, the application of bacteriophage preparations to agricultural produce substantially reduce or eliminate the possibility of food-borne illness through application of a single phage or phage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria are rapidly identified when required, whereas identification of a new effective sanitizer is a much longer process, several years.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier. In some embodiments, a pharmaceutical composition or method disclosed herein treats urinary tract infections (UTI) and/or inflammatory diseases (e.g. inflammatory bowel disease (IBD)). In some embodiments, a pharmaceutical composition or method disclosed herein treats Crohn's disease. In some embodiments, a pharmaceutical composition or method disclosed herein treats ulcerative colitis.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiment, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostats and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried (lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to Ethylenediaminetetraacetic acid (EDTA), citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, xylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin.

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

### Further Embodiments

1. A bacteriophage comprising a CRISPR system comprising:
   (a) a CRISPR array comprising a spacer sequence complementary to a target nucleotide sequence in an Escherichia species; and
   (b) a nucleic acid sequence encoding a CRISPR associated nuclease.
2. The bacteriophage of embodiment 1, wherein the spacer sequence comprises at least about 90%, 91%, 92%, 93%, 94%, 95%, 96%, 97%, 98%, or 99% sequence identity to any one of SEQ ID NOS: 12 or 20-37.
3. The bacteriophage of embodiment 1 or embodiment 2, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
4. The bacteriophage of any one of embodiments 1-3, wherein the CRISPR array further comprises at least one repeat sequence, wherein optionally the at least one repeat sequence is operably linked to the spacer sequence at either its 5′ end or its 3′ end.
5. The bacteriophage of embodiment 4, wherein the repeat sequence comprises at least about 90%, 91%, 92%, 93%, 94%, 95%, 96%, 97%, 98%, or 99% sequence identity to any one of SEQ ID NOs: 13-18.
6. The bacteriophage of any one of embodiments 1-34, wherein the CRISPR array comprises at least about 90%, 91%, 92%, 93%, 94%, 95%, 96%, 97%, 98%, or 99% sequence identity to a sequence selected from SEQ ID NOS: 38-45.
7. The bacteriophage of any one of embodiments 1-6, wherein the target nucleotide sequence comprises a coding sequence.
8. The bacteriophage of any one of embodiments 1-6, wherein the target nucleotide sequence comprises a non-coding or intergenic sequence.
9. The bacteriophage of any one of embodiments 1-6, wherein the target nucleotide sequence comprises all or a part of (e.g., at least 10 contiguous nucleobases) a promoter sequence.
10. The bacteriophage of embodiment 9, wherein the promoter sequence comprises at least about 90%, 91%, 92%, 93%, 94%, 95%, 96%, 97%, 98%, or 99% sequence identity to any one of SEQ ID NOs: 1-11 or 19.
11. The bacteriophage of any one of embodiments 1-10, wherein the target nucleotide sequence comprises all or a part of (e.g., at least 10 contiguous nucleobases) a nucleotide sequence located on a coding strand of a transcribed region of an essential gene, or a non-essential gene.
12. The bacteriophage of embodiment 11, wherein the target nucleotide sequence comprises all of a part of (e.g., at least 10 contiguous nucleobases) a nucleotide sequence located on a coding strand of a transcribed region of an essential gene, and the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
13. The bacteriophage of any one of embodiments 1-12, wherein the CRISPR system comprises a CRISPR-Cpf1 system, a Type II CRISPR-Cas system, or a Type V CRISPR-Cas system.
14. The bacteriophage of any one of embodiments 1-12, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
15. The bacteriophage of embodiment 14, wherein the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, and a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
16. The bacteriophage of embodiment 14, wherein the Cascade complex comprises a Cas5d polypeptide, a Cas8c polypeptide, and a Cas7 polypeptide (Type I-C CRISPR-Cas system).
17. The bacteriophage of any one of embodiments 14-16, wherein the Cascade complex comprises a Pseudomonas aeruginosa Type I-C Cascade complex.
18. The bacteriophage of any one of embodiments 1-17, further comprising a nucleic acid sequence comprising a promoter sequence.
19. The bacteriophage of any one of embodiments 1-18, wherein the bacteriophage infects multiple bacterial strains.
20. The bacteriophage of any one of embodiments 1-19, wherein the bacteriophage is an obligate lytic bacteriophage.
21. The bacteriophage of any one of embodiments 1-20, wherein the bacteriophage is a temperate bacteriophage that is rendered lytic.
22. The bacteriophage of embodiment 21, wherein the temperate bacteriophage is rendered lytic by the removal, replacement, or inactivation of a lysogeny gene.
23. The bacteriophage of any one of embodiments 20-22, wherein the Escherichia species is killed solely by lytic activity of the bacteriophage.
24. The bacteriophage of any one of embodiments 20-22, wherein the Escherichia species is killed solely by activity of the CRISPR system.
25. The bacteriophage of any one of embodiments 20-22, wherein the Escherichia species is killed by lytic activity of the bacteriophage in combination with activity of the CRISPR system.
26. The bacteriophage of embodiment 25, wherein the Escherichia species is killed by the activity of the CRISPR system, independently of the lytic activity of the bacteriophage.
27. The bacteriophage of embodiment 25, wherein the activity of the CRISPR system supplements or enhances the lytic activity of the bacteriophage.
28. The bacteriophage of embodiment 25, wherein the lytic activity of the bacteriophage and the activity of the CRISPR system are synergistic.
29. The bacteriophage of any one of embodiments 20-26, wherein the lytic activity of the bacteriophage, the activity of the CRISPR system, or both, is modulated by a concentration of the bacteriophage.
30. The bacteriophage of any one of embodiments 1-29, wherein the bacteriophage comprises a Tequatrovirus, a Mosigyvirus, a Phapecoctavirus, a Unique Myoviridae, or a Vectrevirus.
31. The bacteriophage of any one of embodiments 1-30, wherein the bacteriophage comprises at least 80% sequence identity to a phage selected from p004K, p00c0, p00ex, p00jc, p00ke, or p5516.
32. The bacteriophage of embodiment 31, wherein the bacteriophage is p004Ke007, p004Ke009, p00c0e030, or p00exe014.
33. The bacteriophage of any one of embodiments 1-32, wherein the nucleic acid sequence is inserted into a non-essential bacteriophage gene.
34. A pharmaceutical composition comprising:
(a) the bacteriophage of any one of embodiments 1-33; and
(b) a pharmaceutically acceptable excipient.
35. The pharmaceutical composition of embodiment 34, wherein the pharmaceutical composition comprises at least two bacteriophage.
36. The pharmaceutical composition of embodiment 34 or embodiment 35, wherein the bacteriophage are from the lineage consisting of a Tequatrovirus, a Mosigyvirus, a Phapecoctavirus, a Unique Myoviridae, or a Vectrevirus.
37. The pharmaceutical composition of embodiment 35 or embodiment 36, wherein the pharmaceutical composition comprises at least six bacteriophage, wherein the bacteriophage comprise p004k, p00c0, p00ex, p00jc, p00ke, and p5516; or p00exe014, p004Ke009, p00jc, p00ke, p00c0e030, and p5516.
38. The pharmaceutical composition of any one of embodiments 34-37, wherein the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof
39. A method of killing an Escherichia species comprising introducing into the Escherichia species genetic material from a bacteriophage comprising a CRISPR system comprising:
(a) a CRISPR array comprising a spacer sequence complementary to target nucleotide sequence in the Escherichia species; and
(b) a nucleic acid sequence encoding a CRISPR associated nuclease.
40. The method of embodiment 39, wherein the spacer sequence comprises at least about 90%, 91%, 92%, 93%, 94%, 95%, 96%, 97%, 98%, or 99% sequence identity to any one of SEQ ID NOS: 12 or 20-37.
41. The method of embodiment 39 or embodiment 40, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
42. The method of any one of embodiments 39-41, wherein the CRISPR array further comprises at least one repeat sequence, wherein optionally the at least one repeat sequence is operably linked to the spacer sequence at either its 5′ end or its 3′ end.
43. The method of embodiment 42, wherein the repeat sequence comprises at least about 90%, 91%, 92%, 93%, 94%, 95%, 96%, 97%, 98%, or 99% sequence identity to any one of SEQ ID NOs: 13-18.
44. The method of any one of embodiments 39-43, wherein the CRISPR array comprises at least about 90%, 91%, 92%, 93%, 94%, 95%, 96%, 97%, 98%, or 99% sequence identity to a sequence selected from SEQ ID NOS: 38-45.
45. The method of any one of embodiments 39-44, wherein the target nucleotide sequence comprises a coding sequence.
46. The method of any one of embodiments 39-44, wherein the target nucleotide sequence comprises a non-coding or intergenic sequence.
47. The method of any one of embodiments 39-46, wherein the target nucleotide sequence comprises all or a part of (e.g., at least 10 contiguous nucleobases) a promoter sequence.
48. The method of embodiment 47, wherein the promoter sequence comprises at least about 90%, 91%, 92%, 93%, 94%, 95%, 96%, 97%, 98%, or 99% sequence identity to any one of SEQ ID NOs: 1-11 or 19.
49. The method of any one of embodiments 39-48, wherein the target nucleotide sequence comprises all or a part of (e.g., at least 10 contiguous nucleobases) a nucleotide sequence located on a coding strand of a transcribed region of an essential gene, or a non-essential gene.
50. The method of embodiment 49, wherein the target nucleotide sequence comprises all of a part of (e.g., at least 10 contiguous nucleobases) a nucleotide sequence located on a coding strand of a transcribed region of an essential gene, and the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
51. The method of any one of embodiments 39-50, wherein the CRISPR system comprises a CRISPR-Cpf1 system, a Type II CRISPR-Cas system, or a Type V CRISPR-Cas system.
52. The method of any one of embodiments 39-50, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
53. The method of embodiment 52, wherein the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
54. The method of embodiment 52, wherein the Cascade complex comprises a Cas5d polypeptide, a Cas8c polypeptide, and a Cas7 polypeptide (Type I-C CRISPR-Cas system).
55. The method of embodiment 54, wherein the Cascade complex comprises a Pseudomonas aeruginosa Type I-C Cascade complex.
56. The method of any one of embodiments 39-55, wherein the CRISPR system further comprises a nucleic acid sequence comprising a promoter sequence.
57. The method of any one of embodiments 39-56, wherein the bacteriophage is an obligate lytic bacteriophage.
58. The method of any one of embodiments 39-57, wherein the bacteriophage is a temperate bacteriophage that is rendered lytic.
59. The method of embodiment 58, wherein the temperate bacteriophage is rendered lytic by the removal, replacement, or inactivation of a lysogeny gene.
60. The method of any one of embodiments 39-59, wherein the Escherichia species is killed solely by activity of the CRISPR system.
61. The method of any one of embodiments 57-60, wherein the Escherichia species is killed by lytic activity of the bacteriophage in combination with activity of the CRISPR system.
62. The method of embodiment 61, wherein the Escherichia species is killed by the activity of the CRISPR system, independently of the lytic activity of the bacteriophage.
63. The method of embodiment 61, wherein the activity of the CRISPR system supplements or enhances the lytic activity of the bacteriophage.
64. The method of embodiment 61, wherein the lytic activity of the bacteriophage and the activity of the CRISPR system are synergistic.
65. The method of any one of embodiments 60-64, wherein the lytic activity of the bacteriophage, the activity of the CRISPR system, or both, is modulated by a concentration of the bacteriophage.
66. The method of any one of embodiments 39-65, wherein the bacteriophage infects multiple bacterial strains.
67. The method of any one of embodiments 39-66, wherein the bacteriophage comprises at least 80% identity to p004K, p00c0, p00ex, p00jc, p00ke, p5516, p004Ke007, p004Ke009, p00c0e030, or p00exe014.
68. The method of any one of embodiments 39-67, wherein the nucleic acid sequence is inserted in place of or adjacent to a non-essential bacteriophage gene.
69. The method of any one of embodiments 39-68, wherein a mixed population of bacterial cells comprises the Escherichia species.
70. A method of treating a disease in an individual in need thereof, the method comprising administering to the individual a bacteriophage comprising a CRISPR system comprising:
(a) a CRISPR array comprising a spacer sequence complementary to target nucleotide sequence in an Escherichia species; and
(b) a nucleic acid sequence encoding a CRISPR associated nuclease.
71. The method of embodiment 70, wherein the spacer sequence comprises at least about 90%, 91%, 92%, 93%, 94%, 95%, 96%, 97%, 98%, or 99% sequence identity to any one of SEQ ID NOS: 12 or 20-37.
72. The method of embodiment 70 or embodiment 71, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
73. The method of embodiment 70 or embodiment 71, wherein the CRISPR array further comprises at least one repeat sequence, wherein optionally the at least one repeat sequence is operably linked to the spacer sequence at either its 5′ end or its 3′ end.
74. The method of embodiment 73, wherein the repeat sequence comprises at least about 90%, 91%, 92%, 93%, 94%, 95%, 96%, 97%, 98%, or 99% sequence identity to any one of SEQ ID NOs: 13-18.
75. The method of any one of embodiments 70-74, wherein the CRISPR array comprises at least about 90%, 91%, 92%, 93%, 94%, 95%, 96%, 97%, 98%, or 99% sequence identity to a sequence selected from SEQ ID NOS: 38-45.
76. The method of any one of embodiments 70-75, wherein the target nucleotide sequence comprises a coding sequence.
77. The method of any one of embodiments 70-75, wherein the target nucleotide sequence comprises a non-coding or intergenic sequence.
78. The method of any one of embodiments 70-77, wherein the target nucleic acid sequence comprises all or a part of (e.g., at least 10 contiguous nucleobases) a promoter sequence.
79. The method of embodiment 78, wherein the promoter sequence comprises at least about 90%, 91%, 92%, 93%, 94%, 95%, 96%, 97%, 98%, or 99% sequence identity to any one of SEQ ID NOs: 1-11 or 19.
80. The method of any one of embodiments 70-79, wherein the target nucleotide sequence comprises all or a part of (e.g., at least 10 contiguous nucleobases) a nucleotide sequence located on a coding strand of a transcribed region of an essential gene, or a non-essential gene.
81. The method of embodiment 80, wherein the target nucleotide sequence comprises all of a part of (e.g., at least 10 contiguous nucleobases) a nucleotide sequence located on a coding strand of a transcribed region of an essential gene, and the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
82. The method of any one of embodiments 70-81, wherein the CRISPR system comprises a CRISPR-Cpf1 system, a Type II CRISPR-Cas system, or a Type V CRISPR-Cas system.
83. The method of any one of embodiments 70-81, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
84. The method of embodiment 83, wherein the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
85. The method of embodiment 84, wherein the Cascade complex comprises a Cas5d polypeptide, a Cas8c polypeptide, and a Cas7 polypeptide (Type I-C CRISPR-Cas system).
86. The method of any one of embodiments 70-85, wherein the CRISPR system further comprises a nucleic acid sequence comprising a promoter sequence.
87. The method of any one of embodiments 70-86, wherein the bacteriophage is an obligate lytic bacteriophage.
88. The method of any one of embodiments 70-86, wherein the bacteriophage is a temperate bacteriophage that is rendered lytic.
89. The method of embodiment 88, wherein the temperate bacteriophage is rendered lytic by the removal, replacement, or inactivation of a lysogeny gene.
90. The method of any one of embodiments 87-89, wherein the Escherichia species is killed solely by activity of the CRISPR system.
91. The method of any one of embodiments 87-89, wherein the Escherichia species is killed by lytic activity of the bacteriophage in combination with activity of the CRISPR system.
92. The method of embodiment 91, wherein the Escherichia species is killed by the activity of the CRISPR system, independently of the lytic activity of the bacteriophage.
93. The method of embodiment 91, wherein the activity of the CRISPR system supplements or enhances the lytic activity of the bacteriophage.
94. The method of embodiment 91, wherein the lytic activity of the bacteriophage and the activity of the CRISPR system are synergistic.
95. The method of any one of embodiments 87-94, wherein the lytic activity of the bacteriophage, the activity of the CRISPR system, or both, is modulated by a concentration of the bacteriophage.
96. The method of any one of embodiments 70-95, wherein the bacteriophage infects multiple bacterial strains.
97. The method of any one of embodiments 70-96, wherein the bacteriophage comprises at least 80% identity to p004K, p00c0, p00ex, p00jc, p00ke, p5516, p004Ke007, p004Ke009, p00c0e030, or p00exe014.
98. The method of any one of embodiments 70-97, wherein the nucleic acid sequence is inserted in place of or adjacent to a non-essential bacteriophage gene.
99. The method of any one of embodiments 70-98, wherein the disease is a bacterial infection.
100. The method of any one of embodiments 70-99, wherein the Escherichia species causing the disease is a drug resistant Escherichia species.
101. The method of embodiment 100, wherein the drug resistant Escherichia species is resistant to at least one antibiotic.
102. The method of any one of embodiments 70-101, wherein the Escherichia species causing the disease is a multidrug resistant Escherichia species.
103. The method of embodiment 102, wherein the multi-drug resistant Escherichia species is resistant to at least one antibiotic.
104. The method of any one of embodiments 101 or 103, wherein the antibiotic comprises a cephalosporin, a fluoroquinolone, a carbapenem, a colistin, an aminoglycoside, vancomycin, streptomycin, or methicillin.
105. The method of any one of embodiments 70-104, wherein the Escherichia species is Escherichia coli.
106. The method of any one of embodiments 70-105, wherein the administering is intra-arterial, intravenous, intraurethral, intramuscular, oral, subcutaneous, inhalation, topical, cutaneous, transdermal, transmucosal, implantation, sublingual, buccal, rectal, vaginal, ocular, otic, or nasal administration or any combination thereof
107. The method of any one of embodiments 70-106, wherein the individual is a mammal.
108. The method of any one of embodiments 70-107, further comprising administering at least one additional bacteriophage.
109. A bacteriophage comprising a nucleic acid sequence encoding a Type I CRISPR-Cas system comprising:
(a) a CRISPR array comprising a spacer sequence complementary to target nucleotide sequence in a Escherichia species;
(b) a Cascade polypeptide comprising Cas5, Cas8c and Cas7; and
(c) a Cas3 polypeptide.
110. The bacteriophage of embodiment 109, wherein the spacer sequence comprises at least about 90%, 91%, 92%, 93%, 94%, 95%, 96%, 97%, 98%, or 99% sequence identity to any one of SEQ ID NOS: 12 or 20-37.
111. The bacteriophage of embodiment 109 or embodiment 110, wherein the spacer sequence comprises a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
112. The bacteriophage of any one of embodiments 109-111, wherein the CRISPR array further comprises at least one repeat sequence, wherein optionally the at least one repeat sequence is operably linked to the spacer sequence at either its 5′ end or its 3′ end.
113. The bacteriophage of embodiment 112, wherein the repeat sequence comprises at least about 90%, 91%, 92%, 93%, 94%, 95%, 96%, 97%, 98%, or 99% sequence identity to any one of SEQ ID NOs: 13-18.
114. The bacteriophage of any one of embodiments 109-113, wherein the CRISPR array comprises at least about 90%, 91%, 92%, 93%, 94%, 95%, 96%, 97%, 98%, or 99% sequence identity to a sequence selected from SEQ ID NOS: 38-45.
115. The bacteriophage of any one of embodiments 109-114, wherein the target nucleotide sequence comprises a coding sequence.
116. The bacteriophage of any one of embodiments 109-114, wherein the target nucleotide sequence comprises a non-coding or intergenic sequence.
117. The bacteriophage of any one of embodiments 109-116, wherein the target nucleotide sequence comprises all or a part of (e.g., at least 10 contiguous nucleobases) a promoter sequence.
118. The bacteriophage of embodiment 117, wherein the promoter sequence comprises at least about 90%, 91%, 92%, 93%, 94%, 95%, 96%, 97%, 98%, or 99% sequence identity to any one of SEQ ID NOs: 1-11 or 19.
119. The bacteriophage of any one of embodiments 109-118, wherein the target nucleotide sequence comprises all or a part of (e.g., at least 10 contiguous nucleobases) a nucleotide sequence located on a coding strand of a transcribed region of an essential gene, or a non-essential gene.
120. The bacteriophage of embodiment 119, wherein the target nucleotide sequence comprises all of a part of (e.g., at least 10 contiguous nucleobases) a nucleotide sequence located on a coding strand of a transcribed region of an essential gene, and the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
121. The bacteriophage of any one of embodiments 109-120, wherein the CRISPR-Cas system further comprises a promoter sequence.
122. The bacteriophage of any one of embodiments 109-121, wherein the bacteriophage is an obligate lytic bacteriophage.
123. The bacteriophage of any one of embodiments 109-121, wherein the bacteriophage is a temperate bacteriophage that is rendered lytic.
124. The bacteriophage of embodiment 123, wherein the temperate bacteriophage is rendered lytic by the removal, replacement, or inactivation of a lysogeny gene.
125. The bacteriophage of any one of embodiments 121-124, wherein the Escherichia species is killed solely by lytic activity of the bacteriophage.
126. The bacteriophage of any one of embodiments 121-124, wherein the Escherichia species is killed solely by activity of the CRISPR system.
127. The bacteriophage of any one of embodiments 121-126, wherein the Escherichia species is killed by lytic activity of the bacteriophage in combination with activity of the CRISPR system.
128. The bacteriophage of embodiment 127, wherein the Escherichia species is killed by the activity of the CRISPR system, independently of the lytic activity of the bacteriophage.
129. The bacteriophage of embodiment 127, wherein the activity of the CRISPR system supplements or enhances the lytic activity of the bacteriophage.
130. The bacteriophage of embodiment 127, wherein the lytic activity of the bacteriophage and the activity of the CRISPR system are synergistic.
131. The bacteriophage of any one of embodiments 125-130, wherein the lytic activity of the bacteriophage, the activity of the CRISPR system, or both, is modulated by a concentration of the bacteriophage
132. The bacteriophage of any one of embodiments 109-131, wherein the bacteriophage infects multiple bacterial strains.
133. The bacteriophage of any one of embodiments 109-132, wherein the bacteriophage comprises at least 80% identity to p004ke007, p004Ke005, p004K, p00c0, p00ex, p00jc, p00ke, or p5516.
134. The bacteriophage of any one of embodiments 109-133, wherein the nucleic acid sequence is inserted into a non-essential bacteriophage gene.
135. A pharmaceutical composition comprising:
(a) the bacteriophage of any one of embodiments 109-134; and
(b) a pharmaceutically acceptable excipient.
136. The pharmaceutical composition of embodiment 135, wherein the pharmaceutical composition comprises at least two bacteriophage.
137. The pharmaceutical composition of embodiment 136, wherein the pharmaceutical composition comprises at last six bacteriophage, wherein each bacteriophage comprises at least 80% sequence identity to p004k, p00c0, p00ex, p00jc, p00ke, and p5516; or p00exe014, p004Ke009, p00jc, p00ke, p00c0e030, and p5516.
138. The pharmaceutical composition of any one of embodiments 135-137, wherein the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof
139. A method of sanitizing a surface in need thereof, the method comprising administering to the surface a bacteriophage comprising a Type I CRISPR system comprising:
(a) a CRISPR array comprising a spacer sequence complementary to target nucleotide sequence in an Escherichia species; and
(b) a nucleic acid sequence encoding a CRISPR associated nuclease.
140. The method of embodiment 139, wherein the surface is a hospital surface, a vehicle surface, an equipment surface, or an industrial surface.
141. The method of embodiment 139 or embodiment 140, wherein the CRISPR system comprises a CRISPR-Cpf1 system, a Type II CRISPR-Cas system, or a Type V CRISPR-Cas system.
142. The method of any one of embodiments 139-141, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
143. The method of embodiment 142, wherein the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
144. The method of embodiment 143, wherein the Cascade complex comprises a Cas5d polypeptide, a Cas8c polypeptide, and a Cas7 polypeptide (Type I-C CRISPR-Cas system).
145. A method of preventing contamination in a food product or a nutritional supplement, the method comprising adding to the food product or the nutritional supplement a bacteriophage comprising a nucleic acid sequence encoding a Type I CRISPR system comprising:
(a) a CRISPR array comprising a spacer sequence complementary to target nucleotide sequence in an Escherichia species; and
(b) a nucleic acid sequence encoding a CRISPR associated nuclease.
146. The method of embodiment 145, wherein the food product or nutritional supplement comprises milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.
147. The method of embodiment 145 or embodiment 146, wherein the CRISPR system comprises a CRISPR-Cpf1 system, a Type II CRISPR-Cas system, or a Type V CRISPR-Cas system.
148. The method of any one of embodiments 145-147, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
149. The method of embodiment 148, wherein the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
150. The method of embodiment 149, wherein the Cascade complex comprises a Cas5d polypeptide, a Cas8c polypeptide, and a Cas7 polypeptide (Type I-C CRISPR-Cas system).
151. A bacteriophage comprising a nucleic acid sequence encoding a Type I CRISPR system comprising:
(a) a CRISPR array comprising spacer sequences complementary to target nucleotide sequence in a Escherichia species, wherein the spacer sequences comprise at least one of SEQ ID NOS: 12 or 20-37; and
(b) a nucleic acid sequence encoding a CRISPR associated nuclease.
152. The bacteriophage of embodiment 151, wherein the spacer sequences comprise a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
153. The bacteriophage of embodiment 151 or embodiment 152, wherein the spacer sequences comprise two or three sequences selected from SEQ ID NOS: 12 or 20-37.
154. The bacteriophage of any one of embodiments 151-153, wherein the CRISPR system comprises a CRISPR-Cpf1 system, a Type II CRISPR-Cas system, or a Type V CRISPR-Cas system.
155. The bacteriophage of any one of embodiments 151-153, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
156. The bacteriophage of embodiment 155, wherein the Cascade complex comprises: (i) a Cas7 polypeptide, a Cas8a1 polypeptide or a Cas8a2 polypeptide, a Cas5 polypeptide, a Csa5 polypeptide, a Cas6a polypeptide, a Cas3′ polypeptide, and a Cas3″ polypeptide having no nuclease activity (Type I-A CRISPR-Cas system); (ii) a Cas6b polypeptide, a Cas8b polypeptide, a Cas7 polypeptide, and a Cas5 polypeptide (Type I-B CRISPR-Cas system); (iii) a Cas5d polypeptide, a Cas8c polypeptide, and a Cas7 polypeptide (Type I-C CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
157. The bacteriophage of embodiment 156, wherein the Cascade complex comprises a Cas5d polypeptide, a Cas8c polypeptide, and a Cas7 polypeptide (Type I-C CRISPR-Cas system).
158. A bacteriophage comprising at least 80% sequence identity to a phage selected from p004K, p00c0, p00ex, p00jc, p00ke, or p5516.
159. The bacteriophage of embodiment 158, wherein the bacteriophage comprises at least 80% identity to p004Ke007, p004Ke009, p00c0e030, or p00exe014.
160. The bacteriophage of embodiment 158 or embodiment 159, further comprising
(a) a CRISPR array comprising one or more spacer sequences complementary to target nucleotide sequence in a Escherichia species;
(b) a nucleic acid encoding a Cascade polypeptide; and
(c) a nucleic acid encoding a Cas3 polypeptide.
161. The bacteriophage of embodiment 160, wherein the one or more spacer sequence comprises at least one of SEQ ID NOs: 12 or 20-37 or at least 90%, 91%, 92%, 93%, 94%, 95%, 96%, 97%, 98%, or 99% sequence identity to any one of SEQ ID NOs: 12 or 20-37.
162. The bacteriophage of embodiment 158, wherein the one or more spacer sequences comprise two or three spacer sequences, wherein each spacer sequence has at least about 90%, 91%, 92%, 93%, 94%, 95%, 96%, 97%, 98%, or 99% sequence identity to any one of SEQ ID NOS: 12 or 20-37.
163. The bacteriophage of any one of embodiments 160-162, wherein the one or more spacer sequences comprise a first spacer sequence and a second spacer sequence, wherein the first spacer sequence is complementary to the target nucleotide sequence in the Escherichia species, and the second spacer sequence is complementary to a second target nucleotide sequence in the Escherichia species and/or a second Escherichia species; optionally wherein the spacer sequence comprises a third spacer sequence, wherein the third spacer sequence is complementary to a third target nucleotide sequence in the Escherichia species, second Escherichia species, and/or third Escherichia species.
164. The bacteriophage of any one of embodiments 158-163, wherein the CRISPR array further comprises at least one repeat sequence.
165. The bacteriophage of embodiment 164, wherein the at least one repeat sequence is operably linked to the spacer sequence at either its 5′ end or its 3′ end.
166. The bacteriophage of embodiment 164 or embodiment 165, wherein the repeat sequence comprises at least about 90%, 91%, 92%, 93%, 94%, 95%, 96%, 97%, 98%, or 99% sequence identity to any one of SEQ ID NOs: 13-18.
167. The bacteriophage of any one of embodiments 158-166, wherein the CRISPR array comprises at least about 90%, 91%, 92%, 93%, 94%, 95%, 96%, 97%, 98%, or 99% sequence identity to a sequence selected from SEQ ID NOS: 38-45.
168. The bacteriophage of any one of embodiments 158-167, wherein the target nucleotide sequence comprises a coding sequence.
169. The bacteriophage of any one of embodiments 158-167, wherein the target nucleotide sequence comprises a non-coding or intergenic sequence.
170. The bacteriophage of any one of embodiments 156-168, wherein the comprising a promoter sequence.
171. The bacteriophage of embodiment 170, wherein the promoter sequence comprises at least about 90%, 91%, 92%, 93%, 94%, 95%, 96%, 97%, 98%, or 99% sequence identity to any one of
172. A bacteriophage modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease.
173. The bacteriophage of embodiment 172, wherein the target bacteria comprises an Escherichia species.
174. The bacteriophage of embodiment 172 or embodiment 173, wherein the modified bacteriophage is viable.
175. The bacteriophage of any one of embodiments 172-174, wherein the modified bacteriophage is lytic.
176. The bacteriophage of any one of embodiments 172-175, wherein the nucleic acid sequence encoding the CRISPR system is a single nucleic acid sequence.
177. The bacteriophage of any one of embodiments 172-175, wherein the nucleic acid sequence encoding the CRISPR system comprises two or more nucleic acid sequences encoding the CRISPR system.
178. The bacteriophage of any one of embodiments 172-177, wherein the CRISPR system, CRISPR array, and/or sequence encoding the CRISPR nuclease is positioned adjacent to (e.g., 0 nucleobases away) or at least one nucleobase away from a bacteriophage coding sequence comprising a promoter and a gene encoding a protein.
179. The bacteriophage of any one of embodiments 172-178, wherein the CRISPR system, CRISPR array, and/or sequence encoding the CRISPR nuclease is positioned adjacent to (e.g., 0 nucleobases away) or at least one nucleobase away from an essential gene of the bacteriophage.
180. The bacteriophage of any one of embodiments 172-179, wherein the CRISPR system comprises a total of about 3000 nucleobases to about 8000 nucleobases.
181. The bacteriophage of any one of embodiments 172-180, wherein the CRISPR system comprises a total of about 4000 nucleobases to about 8000 nucleobases.
182. The bacteriophage of any one of embodiments 172-181, wherein the CRISPR system comprises a total of about 5000 nucleobases to about 8000 nucleobases.
183. The bacteriophage of any one of embodiments 172-182, wherein the bacteriophage DNA is from a p004ke, p00c0, or p00ex phage.
184. The bacteriophage of any one of embodiments 172-183, wherein the bacteriophage is a modified p00ke phage.
185. The bacteriophage of any one of embodiments 172-183, wherein the bacteriophage is a modified p00c0 phage.
186. The bacteriophage of any one of embodiments 172-183, wherein the bacteriophage is a modified p00ex phage.
187. The bacteriophage of any one of embodiments 172-186, wherein the bacteriophage DNA is not essential for viability, does not affect bacteriophage replication, does not affect bacteriophage lysis, does not affect the natural lifestyle of the bacteriophage, or does not affect the functionality of the bacteriophage, or any combination of two or more thereof.
188. The bacteriophage of any one of embodiments 172-187, wherein the CRISPR system is a CRISPR-Cas system.
189. The bacteriophage of any one of embodiments 172-187, wherein the CRISPR system is a CRISPR-Cpf1 system.
190. The bacteriophage of any one of embodiments 172-187, wherein the CRISPR system comprises a CRISPR-Cpf1 system, a Type II CRISPR-Cas system, or a Type V CRISPR-Cas system.
191. The bacteriophage of any one of embodiments 172-187, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
192. The bacteriophage of embodiment 191, wherein the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, and a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
193. The bacteriophage of embodiment 192, wherein the Cascade complex comprises a Cas5d polypeptide, a Cas8c polypeptide, and a Cas7 polypeptide (Type I-C CRISPR-Cas system).
194. The bacteriophage of embodiment 192, wherein the Cascade complex comprises a Pseudomonas aeruginosa Type I-C Cascade complex.
195. The bacteriophage of any one of embodiments 172-187, wherein the CRISPR system comprises Type I CRISPR-Cas components.
196. The bacteriophage of any one of embodiments 172-187, wherein the CRISPR system comprises Type V CRISPR-Cas components.
197. The bacteriophage of any one of embodiments 172-187, wherein the CRISPR system comprises Type II CRISPR-Cas components.
198. The bacteriophage of any one of embodiments 172-187, wherein the CRISPR nuclease comprises a Cas3, or a Cas3′ and a Cas3″ having no nuclease activity.
199. The bacteriophage of any one of embodiments 172-187, wherein the CRISPR nuclease comprises Cpf1.
200. The bacteriophage of any one of embodiments 172-187, wherein the CRISPR nuclease comprises Cas9.
201. The bacteriophage of any one of embodiments 172-200, wherein the spacer sequence comprises at least about 90%, 91%, 92%, 93%, 94%, 95%, 96%, 97%, 98%, or 99% sequence identity to any one of SEQ ID NOS: 12 or 20-37.
202. The bacteriophage of any one of embodiments 172-201, wherein the CRISPR array further comprises at least one repeat sequence.
203. The bacteriophage of embodiment 202, wherein the at least one repeat sequence is operably linked to the spacer sequence at either its 5′ end or its 3′ end.
204. The bacteriophage of embodiment 202 or embodiment 203, wherein the repeat sequence comprises at least about 90%, 91%, 92%, 93%, 94%, 95%, 96%, 97%, 98%, or 99% sequence identity to any one of SEQ ID NOs: 13-18.
205. The bacteriophage of any one of embodiments 172-204, wherein the CRISPR array comprises at least about 90%, 91%, 92%, 93%, 94%, 95%, 96%, 97%, 98%, or 99% sequence identity to a sequence selected from SEQ ID NOS: 38-45.
206. The bacteriophage of any one of embodiments 172-205, wherein the target nucleotide sequence comprises a coding sequence.
207. The bacteriophage of any one of embodiments 172-205, wherein the target nucleotide sequence comprises a non-coding or intergenic sequence.
208. The bacteriophage of any one of embodiments 172-207, wherein the target nucleotide sequence comprises all or a part of (e.g., at least 10 contiguous nucleobases) a promoter sequence.
209. The bacteriophage of embodiment 208, wherein the promoter sequence comprises at least about 90%, 91%, 92%, 93%, 94%, 95%, 96%, 97%, 98%, or 99% sequence identity to any one of
210. The bacteriophage of any one of embodiments 172-209, wherein the target nucleotide sequence comprises all or a part of (e.g., at least 10 contiguous nucleobases) a nucleotide sequence located on a coding strand of a transcribed region of an essential gene, or a non-essential gene.
211. The bacteriophage of embodiment 210, wherein the target nucleotide sequence comprises all of a part of (e.g., at least 10 contiguous nucleobases) a nucleotide sequence located on a coding strand of a transcribed region of an essential gene, and the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK.
212. The bacteriophage of any one of embodiments 172-211, wherein the CRISPR system comprises a nucleic acid sequence comprising a promoter sequence.
213. The bacteriophage of any one of embodiments 172-212, wherein the bacteriophage infects multiple bacterial strains.
214. The bacteriophage of any one of embodiments 172-213, wherein the bacteriophage is an obligate lytic bacteriophage.
215. The bacteriophage of any one of embodiments 172-213, wherein the bacteriophage is a temperate bacteriophage that is rendered lytic.
216. The bacteriophage of embodiment 215, wherein the temperate bacteriophage is rendered lytic by the removal, replacement, or inactivation of a lysogeny gene.
217. The bacteriophage of any one of embodiments 172-216, wherein the target bacteria is killed solely by lytic activity of the bacteriophage.
218. The bacteriophage of any one of embodiments 172-216, wherein the target bacteria is killed solely by activity of the CRISPR system.
219. The bacteriophage of any one of embodiments 172-216, wherein the target bacteria is killed by lytic activity of the bacteriophage in combination with activity of the CRISPR system.
220. The bacteriophage of embodiment 214 or embodiment 215, wherein the target bacteria is killed by the activity of the CRISPR system, independently of the lytic activity of the bacteriophage.
221. The bacteriophage of embodiment 214 or embodiment 215, wherein the activity of the CRISPR system supplements or enhances the lytic activity of the bacteriophage.
222. The bacteriophage of embodiment 214 or embodiment 215, wherein the lytic activity of the bacteriophage and the activity of the CRISPR system are synergistic.
223. The bacteriophage of embodiment 214 or embodiment 215, wherein the lytic activity of the bacteriophage, the activity of the CRISPR system, or both, is modulated by a concentration of the bacteriophage.
224. The bacteriophage of any one of embodiments 172-223, wherein the bacteriophage comprises a Tequatrovirus, a Mosigyvirus, a Phapecoctavirus, a Unique Myoviridae, or a Vectrevirus.
225. The bacteriophage of any one of embodiments 172-224, wherein the bacteriophage comprises at least 80% sequence identity to a phage selected from p004K, p00c0, p00ex, p00jc, p00ke, p5516, p00exe014, p004Ke009, or p00c0e030.
226. The bacteriophage of any one of embodiments 172-225, wherein the bacteriophage is modified from a phage selected from p004K, p00c0, p00ex, p00jc, p00ke, or p5516.
227. A nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOS: 38-45.
228. A nucleic acid sequence having at least 80% identity to SEQ ID NO: 39.
229. The nucleic acid of embodiment 228, having at least 90%, 91%, 92%, 93%, 94%, 95%, 96%, 97%, 98%, or 99% sequence identity to SEQ ID NO: 39.
230. The nucleic acid of embodiment 228, comprising SEQ ID NO: 39.
231. The nucleic acid of any one of embodiments 227-230, comprising SEQ ID NO: 12.
232. The nucleic acid of any one of embodiments 227-231, comprising SEQ ID NO: 25.
233. The nucleic acid of any one of embodiments 227-232, comprising SEQ ID NO: 24.
234. The nucleic acid of any one of embodiments 227-233, comprising SEQ ID NO: 13.
235. A CRISPR array comprising the nucleic acid sequence of any one of embodiments 227-234.
236. The CRISPR array of embodiment 235, further comprising a promoter.
237. The CRISPR array of embodiment 236, wherein the promoter is a promoter from Escherichia, Shigella, Klebsiella, Pseudomonas, or other bacterial species.
238. The CRISPR array of embodiment 236 or embodiment 237, wherein the promoter comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 1-11 or 19.
239. The CRISPR array of embodiment 236 or embodiment 237, wherein the promoter comprises a sequence at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 11.
240. The CRISPR array of any one of embodiments 236-239, wherein the promoter is a rrnB P1 promoter.
241. The CRISPR array of any one of embodiments 227-240, comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 43.
242. A bacteriophage comprising the nucleic acid of any one of embodiments 227-234, or the CRISPR array of any one of embodiments 235-241.
243. The bacteriophage of embodiment 242, wherein the nucleic acid and/or CRISPR array is positioned adjacent to (e.g., 0 nucleobases away) or at least one nucleobase away from a bacteriophage coding sequence comprising a promoter and a gene encoding a protein.
244. The bacteriophage of embodiment 242 or embodiment 243, wherein the nucleic acid and/or CRISPR array is positioned adjacent to (e.g., 0 nucleobases away) or at least one nucleobase away from a bacteriophage coding sequence comprising a promoter and a gene encoding a protein.
245. The bacteriophage of any one of embodiments 242-244, wherein the nucleic acid and/or CRISPR array replaces bacteriophage DNA.
246. The bacteriophage of embodiment 245, wherein the bacteriophage DNA is from a p004ke, p00c0, or p00ex phage.
247. The bacteriophage of embodiment 245 or embodiment 246, wherein the bacteriophage DNA is a modified p00ke phage.
248. The bacteriophage of embodiment 245 or embodiment 247, wherein the bacteriophage DNA is a modified p00c0 phage.
249. The bacteriophage of embodiment 245 or embodiment 247, wherein the bacteriophage DNA is a modified p00ex or phage.
250. The bacteriophage of any one of embodiments 242-249, wherein the bacteriophage DNA is not essential for viability, does not affect bacteriophage replication, does not affect bacteriophage lysis, does not affect the natural lifestyle of the bacteriophage, or does not affect the functionality of the bacteriophage, or any combination of two or more thereof.
251. The bacteriophage of any one of embodiments 242-250, wherein the bacteriophage is viable.
252. The bacteriophage of any one of embodiments 242-251, wherein the bacteriophage infects multiple bacterial strains.
253. The bacteriophage of any one of embodiments 242-252, wherein the bacteriophage is lytic.
254. The bacteriophage of any one of embodiments 242-252, wherein the bacteriophage is an obligate lytic bacteriophage.
255. The bacteriophage of any one of embodiments 242-254, wherein the bacteriophage is a temperate bacteriophage that is rendered lytic.
256. The bacteriophage of embodiment 255, wherein the temperate bacteriophage is rendered lytic by the removal, replacement, or inactivation of a lysogeny gene.
257. The bacteriophage of any one of embodiments 242-256, wherein the bacteriophage comprises a Tequatrovirus, a Mosigyvirus, a Phapecoctavirus, a Unique Myoviridae, or a Vectrevirus.
258. The bacteriophage of any one of embodiments 242-257, wherein the nucleic acid and/or CRISPR array is part of a CRISPR system present in the bacteriophage.
259. The bacteriophage of embodiment 258, wherein the CRISPR system comprises a total of about 3000 nucleobases to about 8000 nucleobases.
260. The bacteriophage of embodiment 258, wherein the CRISPR system comprises a total of about 4000 nucleobases to about 8000 nucleobases.
261. The bacteriophage of embodiment 258, wherein the CRISPR system comprises a total of about 5000 nucleobases to about 8000 nucleobases.
262. The bacteriophage of any one of embodiments 258-261, wherein the CRISPR system is a CRISPR-Cas system.
263. The bacteriophage of any one of embodiments 258-261, wherein the CRISPR system is a CRISPR-Cpf1 system.
264. The bacteriophage of any one of embodiments 258-261, wherein the CRISPR system comprises a CRISPR-Cpf1 system, a Type II CRISPR-Cas system, or a Type V CRISPR-Cas system.
265. The bacteriophage of any one of embodiments 258-261, wherein the CRISPR system comprises a Type I CRISPR-Cas system further comprising a nucleic acid sequence encoding a Cascade polypeptide, optionally wherein the Cascade polypeptide forms a Cascade complex of a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.
266. The bacteriophage of embodiment 265, wherein the Cascade complex comprises: (i) a Cas5d polypeptide, a Cas8c polypeptide, and a Cas7 polypeptide (Type I-C CRISPR-Cas system); (ii) a Cas7 polypeptide, a Cas8a1 polypeptide or a Cas8a2 polypeptide, a Cas5 polypeptide, a Csa5 polypeptide, and a Cas6a polypeptide, a Cas3′ polypeptide, and a Cas3″ polypeptide having no nuclease activity (Type I-A CRISPR-Cas system); (iii) a Cas6b polypeptide, a Cas8b polypeptide, a Cas7 polypeptide, and a Cas5 polypeptide (Type I-B CRISPR-Cas system); (iv) a Cas10d polypeptide, a Csc2 polypeptide, a Csc1 polypeptide, a Cas6d polypeptide (Type I-D CRISPR-Cas system); (v) a Cse1 polypeptide, a Cse2 polypeptide, a Cas7 polypeptide, a Cas5 polypeptide, and a Cas6e polypeptide (Type I-E CRISPR-Cas system); or (vi) a Csy1 polypeptide, a Csy2 polypeptide, a Csy3 polypeptide, and a Csy4 polypeptide (Type I-F CRISPR-Cas system).
267. The bacteriophage of embodiment 266, wherein the Cascade complex comprises a Cas5d polypeptide, a Cas8c polypeptide, and a Cas7 polypeptide (Type I-C CRISPR-Cas system).
268. The bacteriophage of embodiment 266 or embodiment 267, wherein the Cascade complex comprises a Pseudomonas aeruginosa Type I-C Cascade complex.
269. The bacteriophage of any one of embodiments 258-261, wherein the CRISPR system comprises Type I CRISPR-Cas components.
270. The bacteriophage of any one of embodiments 258-261, wherein the CRISPR system comprises Type V CRISPR-Cas components.
271. The bacteriophage of any one of embodiments 258-261, wherein the CRISPR system comprises Type II CRISPR-Cas components.
272. The bacteriophage of any one of embodiments 158-261, wherein the CRISPR system comprises a nucleic acid encoding a nuclease.
273. The bacteriophage of embodiment 272, wherein the nuclease comprises a Cas 3, or a Cas3′ and a Cas3″ having no nuclease activity.
274. The bacteriophage of embodiment 272, wherein the nuclease comprises Cpf1.
275. The bacteriophage of embodiment 272, wherein the CRISPR nuclease comprises Cas9.
276. The bacteriophage of any one of embodiments 242-275, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to a phage selected from p004K, p00c0, p00ex, p00jc, p00ke, or p5516.
277. The bacteriophage of any one of embodiments 242-276, wherein the bacteriophage is modified from a phage selected from p004K, p00c0, p00ex, p00jc, p00ke, or p5516.
278. The bacteriophage of any one of embodiments 242-277, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004Ke009.
279. The bacteriophage of any one of embodiments 242-277, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030.
280. The bacteriophage of any one of embodiments 242-277, wherein the bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014.
281. A composition comprising the bacteriophage of any one of embodiments 1-226 or 242-280, and a wild-type phage.
282. The composition of embodiment 281, wherein the wild-type phage target any one of the bacteria of Table 6.
283. The composition of embodiment 281 or embodiment 282, wherein the wild-type phage comprises p00ke.
284. The composition of any one of embodiments 281-283, wherein the wild-type phage comprises p5516.
285. The composition of any one of embodiments 281-284, wherein the wild-type phage comprises p00jc.
286. A composition comprising the bacteriophage of any one of embodiments 1-226 or 242-280, and a second bacteriophage.
287. The composition of embodiment 286, wherein the second bacteriophage is from the lineage consisting of a Tequatrovirus, a Mosigyvirus, a Phapecoctavirus, a Unique Myoviridae, or a Vectrevirus.
288. The composition of embodiment 286 or embodiment 287, wherein the second bacteriophage is modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease.
289. The composition of any one of embodiments 286-288, wherein at least one of the bacteria of Table 6 is targeted by the second bacteriophage.
290. The composition of embodiment 289, wherein at least 10, 20, 30, 40, 50, 100, 150, 200, 250, or 300 of the bacteria of Table 6 are targeted by the second bacteriophage.
291. The composition of any one of embodiments 286-290, wherein the second bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009.
292. The composition of any one of embodiments 286-290, wherein the second bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030.
293. The composition of any one of embodiments 286-290, wherein the second bacteriophage comprises at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014.
294. A recombinant phage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009.
295. A recombinant phage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030.
296. A recombinant phage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014.
297. A phage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc.
298. A phage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke.
299. A phage comprising at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516.
300. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, and a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, p00exe014, p00jc, p00ke, or p5516.
301. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, and a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00exe014, p00jc, p00ke, or p5516.
302. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, and a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00jc, p00ke, or p5516.
303. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00exe014, p00ke, or p5516.
304. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00exe014, p00jc, or p5516.
305. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, p00c0e030, p00exe014, p00jc, or p00ke.
306. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516.
307. The composition of embodiment 306, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00jc, p00ke, or p5516.
308. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00jc, p00ke, or p5516.
309. The composition of embodiment 308, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00jc, p00ke, or p5516; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00jc, p00ke, or p5516; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00c0e030, p00jc, p00ke, or p5516.
310. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p5516.
311. The composition of embodiment 310, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p5516; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p5516; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p5516.
312. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00jc, or p5516.
313. The composition of embodiment 312, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00jc, or p5516; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00jc, or p5516; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00jc, or p5516.
314. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p004ke009, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p00jc.
315. The composition of embodiment 314, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p00jc; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p00jc; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p00exe014, p00c0e030, p00ke, or p00jc.
316. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc.
317. The composition of embodiment 316, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00jc.
318. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014.
319. The composition of embodiment 318, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00ke, or p00exe014.
320. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc.
321. The composition of embodiment 320, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p5516, p00exe014, or p00jc.
322. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00c0e030, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc.
323. The composition of embodiment 322, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00exe014, or p00jc.
324. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, or p5516.
325. The composition of embodiment 324, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, or p5516; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, or p5516; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00ke, p00c0e030, or p5516.
326. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p5516.
327. The composition of embodiment 326, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p5516; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p5516; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p5516.
328. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00exe014, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p00ke.
329. The composition of embodiment 328, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p00ke; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p00ke; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00jc, p00c0e030, or p00ke.
330. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p5516.
331. The composition of embodiment 330, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p5516; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p5516; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p5516.
332. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00jc, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00ke.
333. The composition of embodiment 332, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00ke; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00ke; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00ke.
334. A composition comprising a first phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p00ke, a second phage having at least 80%, 81%, 82%, 83%, 84%, 85%, 86%, 87%, 88%, 89%, 90%, 91%, 92%, 93%, 94%, 95%, 96%, 97%, 98%, or 99% sequence identity to p5516, and a third phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00jc.
335. The composition of embodiment 334, comprising a fourth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00jc; optionally comprising a fifth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00jc; and optionally comprising a sixth phage having at least 80%, 81%, 82%, 83%, 84%, 85%, 86%, 87%, 88%, 89%, 90%, 91%, 92%, 93%, 94%, 95%, 96%, 97%, 98%, or 99% identity to p004ke009, p00exe014, p00c0e030, or p00jc.
336. The composition of any one of embodiments 307, 309, 311, 313, 315, 317, 319, 321, 323, 325, 327, 329, 331, 333 or 335, wherein the fourth phage is different from the third page.
337. The composition of any one of embodiments 307, 309, 311, 313, 315, 317, 319, 321, 323, 325, 327, 329, 331, 333, 335, or 336, wherein the fifth phage is present, and the fifth phage is different from the third and fourth phage.
338. The composition of any one of embodiments 307, 309, 311, 313, 315, 317, 319, 321, 323, 325, 327, 329, 331, 333 or 335-337, wherein the sixth phage is present, and the sixth phage is different from the third, fourth, and fifth phage.
339. A pharmaceutical composition comprising:
(a) the bacteriophage of any one of embodiments 1-226, 242-280, or 294-299 or the composition of any one of embodiments 281-293 or 300-338; and
(b) a pharmaceutically acceptable excipient.
340. The pharmaceutical composition of embodiment 339, wherein the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.
341. A method of killing bacteria comprising introducing into the bacteria genetic material from the bacteriophage of any one of embodiments 1-226, 242-280, or 294-299.
342. A method of killing bacteria comprising introducing into the bacteria genetic material from a bacteriophage from the composition of any one of embodiments 281-293 or 300-338.
343. A method of treating a disease in an individual in need thereof, the method comprising administering to the individual the bacteriophage of any one of embodiments 1-226, 242-280, or 294-299.
344. A method of treating a disease in an individual in need thereof, the method comprising administering to the individual the composition of any one of embodiments 281-293 or 300-338.
345. The method of embodiment 343 or embodiment 344, wherein the disease is a bacterial infection.
346. The method of any one of embodiments 343-345, wherein the bacteria is Escherichia.
347. A method of sanitizing a surface in need thereof, the method comprising administering to the surface the bacteriophage of any one of embodiments 1-226, 242-280, or 294-299.
348. A method of sanitizing a surface in need thereof, the method comprising administering to the surface the composition of any one of embodiments 281-293 or 300-338.
349. The method of embodiment 347 or embodiment 348, wherein the surface comprises Escherichia.
350. A method of preventing bacterial contamination in a food product or a nutritional supplement, the method comprising adding to the food product or the nutritional supplement the bacteriophage of any one of embodiments 1-226, 242-280, or 294-299.
351. A method of preventing bacterial contamination in a food product or a nutritional supplement, the method comprising adding to the food product or the nutritional supplement the composition of any one of embodiments 281-293 or 300-338.
352. The method of embodiment 350 or embodiment 351, wherein the bacteria is Escherichia.
353. A method of killing bacteria comprising contacting the bacteria with a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
354. A method of killing bacteria comprising contacting the bacteria with a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
355. A method of killing bacteria comprising contacting the bacteria with a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
356. A method of killing bacteria comprising contacting the bacteria with a composition comprising p00jc.
357. A method of killing bacteria comprising contacting the bacteria with a composition comprising p00ke.
358. A method of killing bacteria comprising contacting the bacteria with a composition comprising p5516.
359. A method of treating a disease in an individual in need thereof, the method comprising administering to the individual a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
360. A method of treating a disease in an individual in need thereof, the method comprising administering to the individual a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
361. A method of treating a disease in an individual in need thereof, the method comprising administering to the individual a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
362. A method of treating a disease in an individual in need thereof, the method comprising administering to the individual a composition comprising p00jc.
363. A method of treating a disease in an individual in need thereof, the method comprising administering to the individual a composition comprising p00ke.
364. A method of treating a disease in an individual in need thereof, the method comprising administering to the individual a composition comprising p5516.
365. The method of any one of embodiments 359-364, wherein the disease is a bacterial infection.
366. A method of sanitizing a surface in need thereof, the method comprising administering to the surface a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
367. A method of sanitizing a surface in need thereof, the method comprising administering to the surface a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
368. A method of sanitizing a surface in need thereof, the method comprising administering to the surface a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
369. A method of sanitizing a surface in need thereof, the method comprising administering to the surface a composition comprising p00jc.
370. A method of sanitizing a surface in need thereof, the method comprising administering to the surface a composition comprising p00ke.
371. A method of sanitizing a surface in need thereof, the method comprising administering to the surface a composition comprising p5516.
372. A method of preventing bacterial contamination in a food product or a nutritional supplement, the method comprising adding to the food product or the nutritional supplement a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00jc.
373. A method of preventing bacterial contamination in a food product or a nutritional supplement, the method comprising adding to the food product or the nutritional supplement a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p00ke.
374. A method of preventing bacterial contamination in a food product or a nutritional supplement, the method comprising adding to the food product or the nutritional supplement a composition comprising a bacteriophage at least 80%, 81%, 82%, 83%, 84%, 85%, 86%, 87%, 88%, 89%, 90%, 91%, 92%, 93%, 94%, 95%, 96%, 97%, 98%, or 99% identity to p5516.
375. A method of preventing bacterial contamination in a food product or a nutritional supplement, the method comprising adding to the food product or the nutritional supplement a composition comprising p00jc.
376. A method of preventing bacterial contamination in a food product or a nutritional supplement, the method comprising adding to the food product or the nutritional supplement a composition comprising p00ke.
377. A method of preventing bacterial contamination in a food product or a nutritional supplement, the method comprising adding to the food product or the nutritional supplement a composition comprising p5516.
378. The method of any one of embodiments 366-377, wherein the bacteria is Escherichia.
379. A method of killing a plurality of bacteria, the method comprising combining the plurality of bacteria with a first bacteriophage and a second bacteriophage, wherein the first bacteriophage targets a first subset of the plurality of bacteria, and the second bacteriophage targets the second subset of the plurality of bacteria.
380. A method of treating a disease in an individual comprising a plurality of bacteria, the method comprising administering to the individual a first bacteriophage and a second bacteriophage, wherein the first bacteriophage targets a first subset of the plurality of bacteria, and the second bacteriophage targets the second subset of the plurality of bacteria.
381. The method of embodiment 380, wherein the disease is a bacterial infection.
382. A method of sanitizing a surface comprising a plurality of bacteria, the method comprising administering to the surface a first bacteriophage and a second bacteriophage, wherein the first bacteriophage targets a first subset of the plurality of bacteria, and the second bacteriophage targets the second subset of the plurality of bacteria.
383. A method of preventing bacterial contamination from a plurality of bacteria in a food product or a nutritional supplement, the method comprising adding to the food product or the nutritional supplement a first bacteriophage and a second bacteriophage, wherein the first bacteriophage targets a first subset of the plurality of bacteria, and the second bacteriophage targets the second subset of the plurality of bacteria.
384. The method of any one of embodiments 379-383, wherein targeting the first subset of the plurality of bacteria comprises infecting the first subset of the plurality of bacteria, and targeting the second subset of the plurality of bacteria comprises infecting the second subset of the plurality of bacteria.
385. The method of any one of embodiments 379-384, wherein the first subset is different from the second subset.
386. The method of any one of embodiments 379-385, wherein the plurality of bacteria comprises two or more bacteria of Table 6.
387. The method of any one of embodiments 379-386, wherein the plurality of bacteria comprises at least 50, 100, 150, 200, 250, 300, or 350 bacteria of Table 6.
388. The method of any one of embodiments 379-387, wherein the first bacteriophage comprises the bacteriophage of any one of embodiments 1-226, 242-280, or 294-299.
389. The method of any one of embodiments 379-388, wherein the second bacteriophage comprises the bacteriophage of any one of embodiments 1-226, 242-280, or 294-299.
390. The method of any one of embodiments 379-387, wherein the first bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00jc.
391. The method of any one of embodiments 379-387, wherein the first bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00ke
392. The method of any one of embodiments 379-387, wherein the first bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p5516.
393. The method of any one of embodiments 379-387, wherein the first bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p004Ke009.
394. The method of any one of embodiments 379-387, wherein the first bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00c0e030.
395. The method of any one of embodiments 379-387, wherein the first bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00exe014.
396. The method of any one of embodiments 379-388 or 390-395, wherein the second bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00jc.
397. The method of any one of embodiments 379-388 or 390-395, wherein the second bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00ke.
398. The method of any one of embodiments 379-388 or 390-395, wherein the second bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p5516.
399. The method of any one of embodiments 379-388 or 390-395, wherein the second bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p004Ke009.
400. The method of any one of embodiments 379-388 or 390-395, wherein the second bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00c0e030.
401. The method of any one of embodiments 379-388 or 390-395, wherein the second bacteriophage comprises a phage at least 80%, 81%, 82%, 83%, 84%, 85%, 86%, 87%, 88%, 89%, 90%, 91%, 92%, 93%, 94%, 95%, 96%, 97%, 98%, or 99% identical to p00exe014.

### EXAMPLES

### Example 1: Engineered Phage Used in this Application

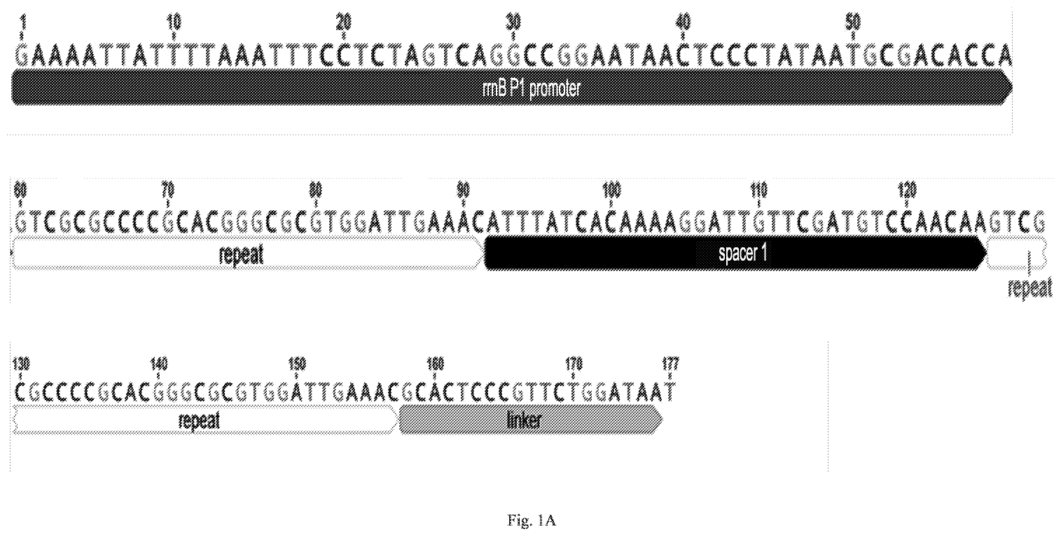

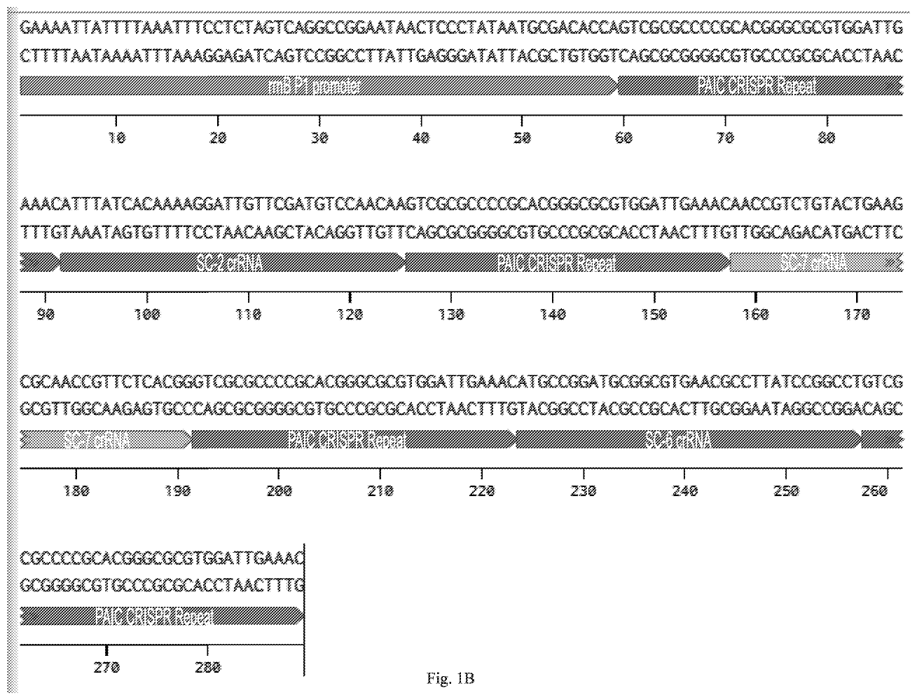

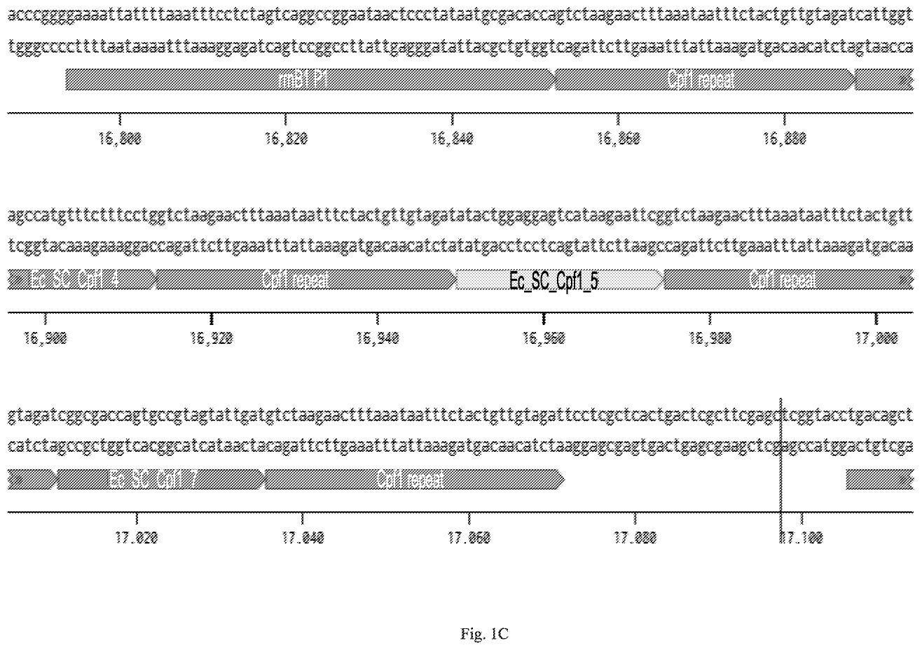

Bacteriophages were engineered to contain a crArray and Cas construct. Table 1 depicts the components of the phages used in the following application. Table 2 depicts the sequences of the promoters used to drive expression of both the crArray and the Cas promoter. Table 3 depicts the sequence of the spacer sequence in the crArray used to target specific sites. Table 4 depicts the repeat sequences. Further, FIG. 1A depicts the sequence and alignment of the preliminary crArray5 (SC2) used in the following examples. FIG. 1B provides the full array sequence and alignment for final cocktail phages.

Cpf1 systems and sequences are provided herein. Exemplary pJC_Cpf1 array sequence and alignment is detailed in FIG. 1C. Cpf1 sequence elements are also listed in Tables 1-4 and 23.

TABLE 1
Components of phage

| | | | Patent | | | | |
|---|---|---|---|---|---|---|---|
| Item | Strain | | Deposit | crArray | crArray | Cas | Cas |
| Description | name | Genera | Number* | Promoter | ID | Promoter | ID |
| Engineered | p004Ke007 | Tequatrovirus | N/A | rrnB P1 | (SC2) | Bba_J23109 | PaIC |
| Bacteriophage | | | | | (SEQ ID | | |
| | | | | | NO: 42) | | |
| Engineered | p004Ke009 | Tequatrovirus | PTA- | rrnB P1 | PAIC | Bba_J23109 | PaIC |
| Bacteriophage | | | 127150 | | array 2 | | |
| | | | | | (FIG. 1B) | | |
| WT | p004K | Tequatrovirus | PTA- | N/A | N/A | N/A | N/A |
| Bacteriophage | | | 127149 | | | | |
| Engineered | p00c0e030 | Mosigvirus | PTA- | rrnB P1 | PAIC | Bba_J23109 | PaIC |
| Bacteriophage | | | 127144 | | array 2 | | |
| | | | | | (FIG. 1B) | | |
| WT | p00c0 | Mosigvirus | PTA- | N/A | N/A | N/A | N/A |
| Bacteriophage | | | 127143 | | | | |
| Engineered | p00exe014 | Tequatrovirus | PTA- | rrnB P1 | PAIC | Bba_J23109 | PaIC |
| Bacteriophage | | | 127146 | | array 2 | | |
| | | | | | (FIG. 1B) | | |
| WT | p00ex | Tequatrovirus | PTA- | N/A | N/A | N/A | N/A |
| Bacteriophage | | | 127145 | | | | |
| WT | p00jc | Phapecoctavirus | PTA- | N/A | N/A | N/A | N/A |
| Bacteriophage | | | 127147 | | | | |
| WT | p00ke | Unique | PTA- | N/A | N/A | N/A | N/A |
| Bacteriophage | | Myoviridae | 127148 | | | | |
| WT | p5516 | Vectrevirus | PTA- | N/A | N/A | N/A | N/A |
| Bacteriophage | | | 127151 | | | | |
| Engineered | p00jc | Phapecoctavirus | N/A | rrnB P1 | Cpf1 | Bba_J23109 | Cpf1 |
| Bacteriophage | | | | | array | | |
| | | | | | (SEQ ID | | |
| | | | | | NO: 44) | | |

*Select phage were deposited with the ATCC Oct. 21, 2021.

TABLE 2
Promoter sequences

| SEQ | | | |
|---|---|---|---|
| ID | | | |
| NO | Promoter | Source | Sequence |
| 1 | ACR | phage | ACAAGCGGCACATTGTGCCTATTGCGAATTAGGCAC |
| | | genome | AATGTGCCTAATCTAACGTCATGCCAGCCACAACGG |
| | | | CGAGGCGCCAAGAAGGATAGAAGCC |
| 2 | BBa_J23109 | BioBrick | TTTACAGCTAGCTCAGTCCTAGGGACTGTGCTAGC |
| 3 | endogenous | P. | GATTTTTTTCGGGTGAGGTTGCGGGCTGTTCGGTAG |
| | (promoter + | aeruginosa | GTTTATAAACACTGCTATCCAAAGCTATGGACACGC |
| | RBS) | genome | TCGGCTACGAGAACAGTTGGCGTGATGGCCTCTAG |
| | | | CAATTAGATTGTTATGCGACATCCGCAGACTTGGCA |
| | | | GGGAGCGCACCT |
| 4 | BBa_J23106 | BioBrick | TTTACGGCTAGCTCAGTCCTAGGTATAGTGCTAGC |
| | P16 | P. | ATCCGAGGGATACGGGCCTTGTCAGCACGGTGTTGC |
| | (promoter + | aeruginosa | TAATGAGAGCCTTTGCCCGGGCAATAGTACGGGCAG |
| | RBS) | genome | TGTGTAGCGGATTGAAAGACGCTGAATCACTGACA |
| | | | GGCATGAAGACTATCGATAGAGTCTGATAGTGTCGC |
| | | | CGCCGCACAGCGGATAGAGTCCACAGTCATTGAAG |
| | | | TGTTAATCCGCGATCAAGCTC |
| 6 | gp105 | phage | GACCTAGCTTTTATAGCGGGTTTCGTGGTTTATAGCC |
| | (promoter + | genome | CATTGAAAAAAATCTCACATCTATATCACAGGTGTG |
| | RBS) | | CACTCGTTCCCGAAAGGTTCTGAGTCTACTTGATCA |
| | | | AGTATTGAAATACCATCGTAAAGGAAAAAGACATGT |
| | | | CTATTCGTGATAGCGAAAACAACAACGGCCAACAG |
| | | | CAGCAGACCGCGCAAACTGCCGCCCCCGCCCCGCA |
| | | | A |
| 7 | gp245 | phage | TTCAATTTAAGTAGTAACGAGGTCAGCCCGGAATCT |
| | (promoter + | genome | TTGGGTATTCTTAAGGTATTTCTGACTCAGTGTGGTT |
| | RBS) | | GGGACAGCTTCACTGTACATTGCACTGGATTTGTTA |
| | | | ATTTCTTATACCGGGGCACCATGGGCAGCAAATCGT |
| | | | GTTACGAATTCCGTCTAACCAATAAGCGAGCTAAAT |
| | | | A |
| 8 | Pamp | plasmid | CGCGGAACCCCTATTTGTTTATTTTTCTAAATACATT |
| | | | CAAATATGTATCCGCTCATGAGACAATAACCCTGATA |
| | | | AATGCTTCAATAATATTGAAAAAGGAAGAGT |
| 9 | Plpp | P. | CTTCAAGAATTCGTATTGACCCCATAGACAGCTTCG |
| | (promoter + | aeruginosa | TCGACGCCCGTCCCGGCCCCCTTGGGCTTGCCGGA |
| | RBS) | genome | CGGCTTATGTCATGATGGCGCCACCCTCGCAGGTTC |
| | | | AAGGCCGGCTTTCTTCCTCTATGAACAAATCCCTTG |
| | | | CGCTGACTACGTAATCAC |
| 10 | Ptat | P. | CTTCAAGAATTCGGGGTATTCCTGATCCTGCGCCGC |
| | (promoter + | aeruginosa | TAGCGCCGCGCACGGCCACTAGGCCCGCGCCGATA |
| | RBS) | genome | GCCAGTCGCGCTCCCGGCTGGCACACTACTCCCATT |
| | | | TCCGCCGGAAACGCGCGCAACGTACCGGCAACGAA |
| | | | CGTGGAAAGACCATGAAAGACTGGCTGGATGAGAT |
| | | | TCACTGGAACGCCGTGACCTACGTATGCAC |
| 11 | rrnB P1 | E. coli | GAAAATTATTTTAAATTTCCTCTAGTCAGGCCGGAAT |
| | | | AACTCCCTATAATGCGACACCA |
| 19 | BBa_J23109 | BioBrick | TTTACAGCTAGCTCAGTCCTAGGGACTGTGCTAGCA |
| | (promoter + | | TTAAAGAGGAGAAAATG |
| | RBS) | | |

TABLE 3
Spacer and array sequences

| Array | Spacer | SEQ ID | |
|---|---|---|---|
| ID | Target | NO | Sequence |
| SC2 | ppSa | 12 | ATTTATCACAAAAGGATTGTTCGATGTCCAACAA |
| SC1 | rpoD | 20 | CGGCAGATGGTCATTGACCTCGGCATAGGTCAGA |
| SC3 | cydB | 21 | ATAATCGATCATTTGACGACTCCTGTCTTAGCGT |
| SC4 | ihFa/pheT | 22 | TGACATTTCAGCTTTTGTAAGCGCCATAGGTTCA |
| SC5 | raiA | 23 | GCCTTTGTGCTGCAGTTTATTGAGCTGCCGTTCC |
| SC6 | intergenic | 24 | ATGCCGGATGCGGCGTGAACGCCTTATCCGGCCT |
| | conserved | | |
| | repeat | | |
| SC7 | rpmH | 25 | AACCGTCTGTACTGAAGCGCAACCGTTCTCACGG |
| SC8 | accB | 26 | GTAAGATTAAAAAACTGATCGAGCTGGTTGAAGA |
| SC9 | gyrB | 27 | TTATGACTCCTCCAGTATCAAAGTCCTGAAAGGG |
| SC10 | rplS | 28 | GTAACCGCGGTCTGCACTCTGCATTCACTGTTCG |
| SC11 | leuS | 29 | CCTATCCTTCTGGTCGACTACACATGGGCCACGT |
| SC12 | rpsD | 30 | TGATCTGTCTGCGGACATTAACGAACACCTGATC |
| SC13 | rplE | 31 | TGTCATGCAAGTCCCTCGGGTCGAGAAGATCACC |
| SC14 | rplR | 32 | CAATATCATGGTCGTGTCCAGGCACTGGCAGATG |
| SC15 | rpsU | 33 | TATGAAAAACCGACTACCGAACGTAAGCGCGCTA |
| SC16 | fusA | 34 | TGAACAACGAAATCATCCTGGTAACCTGTGGTTC |
| Cpf1_4 | UMP | 35 | CATTGGTAGCCATGTTTCTTTCCTG |
| | kinase | | |
| Cpf2_5 | DNA | 36 | ATACTGGAGGAGTCATAAGAATTCG |
| | gyrase | | |
| | subunit B | | |
| Cpf3_7 | 30s | 37 | CGGCGACCAGTGCCGTAGTATTGAT |
| | ribosomal | | |
| | protein | | |
| SC2 | ppSa | 38 | GTCGCGCCCCGCACGGGCGCGTGGATTGAAACATTT |
| array | | | ATCACAAAAGGATTGTTCGATGTCCAACAAGTCGC |
| | | | GCCCCGCACGGGCGCGTGGATTGAAAC |
| PAIC | ppSa, | 39 | GTCGCGCCCCGCACGGGCGCGTGGATTGAAACATTT |
| array 2 | intergenic | | ATCACAAAAGGATTGTTCGATGTCCAACAAGTCGC |
| (4 | conserved | | GCCCCGCACGGGCGCGTGGATTGAAACAACCGTCT |
| repeats) | repeat, and | | GTACTGAAGCGCAACCGTTCTCACGGGTCGCGCCCC |
| | rpmH | | GCACGGGCGCGTGGATTGAAACATGCCGGATGCGG |
| | | | CGTGAACGCCTTATCCGGCCTGTCGCGCCCCGCACG |
| | | | GGCGCGTGGATTGAAAC |
| PAIC | ppSa, | 40 | ATTTATCACAAAAGGATTGTTCGATGTCCAACAAGT |
| array 2 | intergenic | | CGCGCCCCGCACGGGCGCGTGGATTGAAACAACCG |
| (2 | conserved | | TCTGTACTGAAGCGCAACCGTTCTCACGGGTCGCGC |
| repeats) | repeat, and | | CCCGCACGGGCGCGTGGATTGAAACATGCCGGATG |
| | rpmH | | CGGCGTGAACGCCTTATCCGGCCT |
| Cpf1 | UMP | 41 | GTCTAAGAACTTTAAATAATTTCTACTGTTGTAGAT |
| array | kinase, | | CATTGGTAGCCATGTTTCTTTCCTGGTCTAAGAACTT |
| | DNA | | TAAATAATTTCTACTGTTGTAGATATACTGGAGGAG |
| | gyrase | | TCATAAGAATTCGGTCTAAGAACTTTAAATAATTTC |
| | subunit B, | | TACTGTTGTAGATCGGCGACCAGTGCCGTAGTATTG |
| | and 30s | | ATGTCTAAGAACTTTAAATAATTTCTACTGTTGTAG |
| | ribosomal | | AT |
| | protein | | |
| SC2 | ppSa | 42 | GAAAATTATTTTAAATTTCCTCTAGTCAGGCCGGAA |
| array | | | TAACTCCCTATAATGCGACACCAGTCGCGCCCCGCA |
| with | | | CGGGCGCGTGGATTGAAACATTTATCACAAAAGGA |
| example | | | TTGTTCGATGTCCAACAAGTCGCGCCCCGCACGGGC |
| promoter | | | GCGTGGATTGAAAC |
| PAIC | ppSa, | 43 | GAAAATTATTTTAAATTTCCTCTAGTCAGGCCGGAA |
| array 2 | intergenic | | TAACTCCCTATAATGCGACACCAGTCGCGCCCCGCA |
| with | conserved | | CGGGCGCGTGGATTGAAACATTTATCACAAAAGGA |
| example | repeat, and | | TTGTTCGATGTCCAACAAGTCGCGCCCCGCACGGGC |
| promoter | rpmH | | GCGTGGATTGAAACAACCGTCTGTACTGAAGCGCA |
| (FIG. 1B) | | | ACCGTTCTCACGGGTCGCGCCCCGCACGGGCGCGTG |
| | | | GATTGAAACATGCCGGATGCGGCGTGAACGCCTTA |
| | | | TCCGGCCTGTCGCGCCCCGCACGGGCGCGTGGATTG |
| | | | AAAC |
| Cpf1 | UMP | 44 | GAAAATTATTTTAAATTTCCTCTAGTCAGGCCGGAA |
| array | kinase, | | TAACTCCCTATAATGCGACACCAGTCTAAGAACTTT |
| with | DNA | | AAATAATTTCTACTGTTGTAGATCATTGGTAGCCAT |
| example | gyrase | | GTTTCTTTCCTGGTCTAAGAACTTTAAATAATTTCTA |
| promoter | subunit B, | | CTGTTGTAGATATACTGGAGGAGTCATAAGAATTCG |
| | and 30s | | GTCTAAGAACTTTAAATAATTTCTACTGTTGTAGAT |
| | ribosomal | | CGGCGACCAGTGCCGTAGTATTGATGTCTAAGAACT |
| | protein | | TTAAATAATTTCTACTGTTGTAGAT |

TABLE 4
Repeat Sequences

| Repeat | SEQ ID NO: | Sequence |
|---|---|---|
| Repeat 1 | 13 | GTCGCGCCCCGCACGGGCGCGTGGATTGAAAC |
| Repeat 2 | 14 | GTCGCGCCCCGCACGGGCGCGTGGAGTGAAAG |
| Repeat 3 | 15 | GTCGCGCCCCGCACGGGTGCGTGGATTGAAAC |
| Repeat 4 | 16 | GTCGCGCCCCGCATGGGCGCGTGGATTGAACA |
| Repeat 5 | 17 | GTCGCGCCCTACGCGGGCGCGTGGAGTGAAAG |
| Cpf1 repeat | 18 | GTCTAAGAACTTTAAATAATTTCTACTGTTGTAGAT |

### Example 2: Targeting crArrays in E. coli

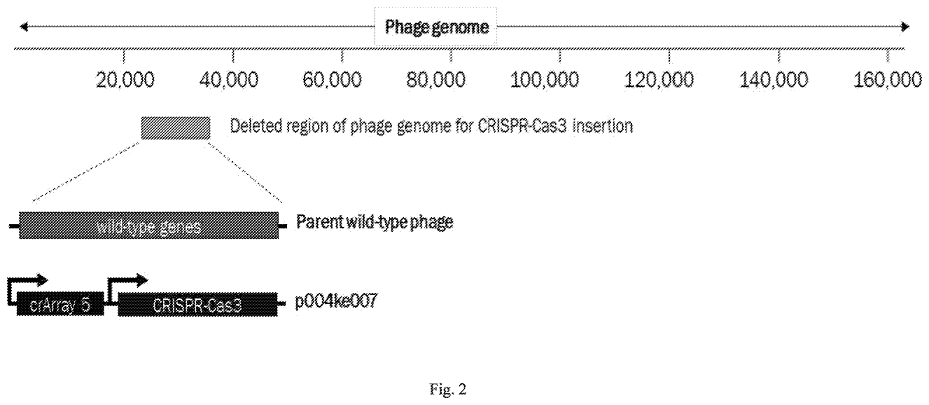

FIG. 2 shows a schematic representation of the genome of wild type phage p004k and its engineered variant p004ke007. The bar below the genome axis indicates the region of the genome that was removed and replaced. The schematic below the phage genome illustrates the DNA that was used to replace WT phage genes in the deleted region.

E. coli phage p004k-wt (wild type) and p004ke007 (targeting SC2+Cas system) were mixed with the indicated bacterial strain while the bacteria was in logarithmic growth and plated onto LB agar 3 hours post inoculation. The ratio of phage to bacteria was altered by performing a dilution series of the phage, so that the amount of bacteria remained constant in each spot but the amount of phage changed. The relative ratio of the phage and bacteria shifted over the course of the experiment as the bacteria replicate and succumb to the phage, which is why an MOI is not listed. The label at the top of each set of images denotes the strain shown.

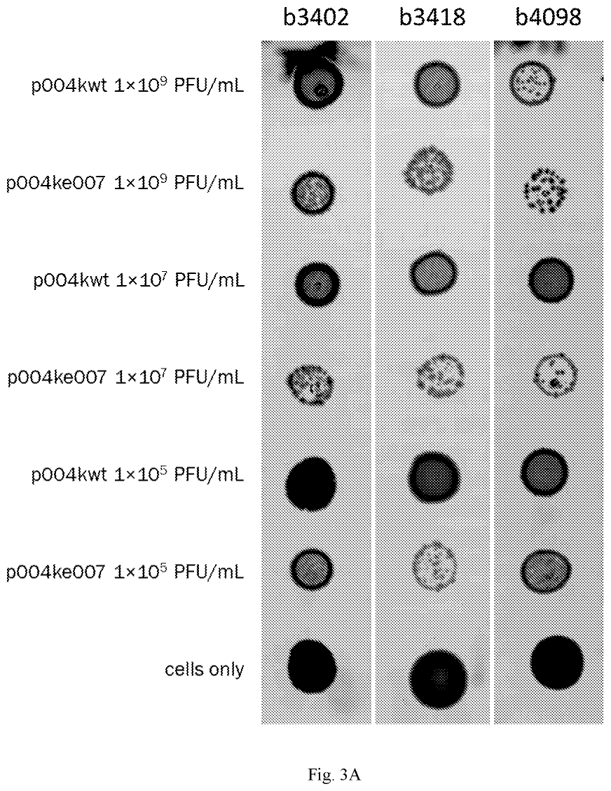

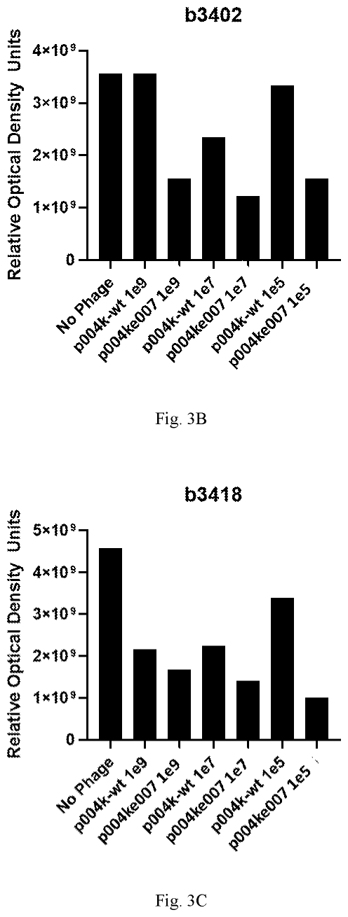

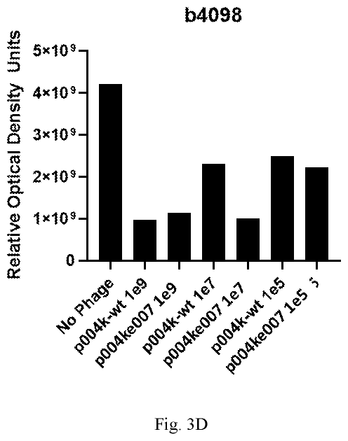

In this assay, the phage was mixed 1:1 with the indicated bacterial culture in mid-logarithmic growth to obtain the final phage titers listed on the left side of the image. The bacteria-phage mixture was incubated for 3 hours and then 2 ul of the culture was spotted onto LB plates, as depicted in FIG. 3A. Bacteria that survived the phage replicate to form visible colonies, so fewer bacteria means better phage kill. In this assay, the engineered phage appeared to kill the bacteria better than wildtype at all dilutions, but the 1×10⁷ dilution was the most visually striking for each E. coli strain. FIGS. 3B-3D show quantification of the images in FIG. 3A. The quantification was determined by comparing the relative optical density of each spot (essentially how dark each spot is, with a darker spot being indicative of more cell growth). Together, these data show that the engineered phage containing a Cas systems and crArray targeting the bacterial genome has improved kill over the wild type phage parent in multiple strains. These data demonstrate that phage expressing exogenous Cas systems improve the phage against diverse human pathogens.

### Example 3: Designing and Validating Spacer Sequences to Target a Target Bacterium Spacer Design

A spacer sequence is designed using the following protocol. First, suitable search set of representative genomes for the organism/species/target of interest are acquired. Examples of suitable databases include NCBI genbank and the PATRIC (Pathosystems Resource Integration Center) database. The genomes are downloaded in bulk via FTP (File Transfer Protocol) servers, enabling rapid and programmatic dataset acquisition.

The genomes are searched with relevant parameters to locate suitable spacer sequences. The genomes can be read from start to end, in both the forward and reverse complement orientations, to locate contiguous stretches of DNA that contain a PAM (Protospacer Adjacent Motif) site. The spacer sequence will be the N-length DNA sequence 3′ adjacent to the PAM site, where N is specific to the Cas system of interest and is generally known ahead of time. Characterizing the PAM sequence and spacer sequences are generally performed during the discovery and initial research of a Cas system. Every observed PAM-adjacent spacer can be saved to a file and/or database for downstream use.

Next, the quality of a spacer for use in a CRISPR engineered phage is determined using the following process. First, each observed spacer can be evaluated to determine how many of the evaluated genomes they are present in. The observed spacers can additionally be evaluated to see how many times they may occur in each given genome. Spacers that occur in more than one location per genome can be advantageous because the Cas system may not be able to recognize the target site if a mutation occurs, and each additional “backup” site increases the likelihood that a suitable, non-mutated target location will be present. The observed spacers can be evaluated to determine whether they occur in functionally annotated regions of the genome. If such information is available, the functional annotations can be further evaluated to determine whether those regions of the genome are “essential” for the survival and function of the organism. Focusing on spacers that occur in all, or nearly all, evaluated genomes of interest (>=99) ensures broad applicability to justify the spacer selection. Provided a large selection pool of conserved spacers exists, preference may be given to spacers that occur in regions of the genome that have known function, with higher preference given if those genomic regions are “essential” for survival and occur more than 1 time per genome.

### Spacer Validation

The identified spacer sequences can then be validated by completing the following procedure. First, a plasmid that replicates in the organism(s) of interest and has a selectable marker (e.g. an antibiotic-resistance gene) is identified. The genes encoding the Cas system are inserted into the plasmid such that they will be expressed in the organism of interest. Upstream of the Cas system, a promoter is included that is recognized by the organism of interest to drive expression of the Cas system. Between the promoter and the Cas system, a ribosomal binding site (RBS) is included that is recognized by the organism of interest.

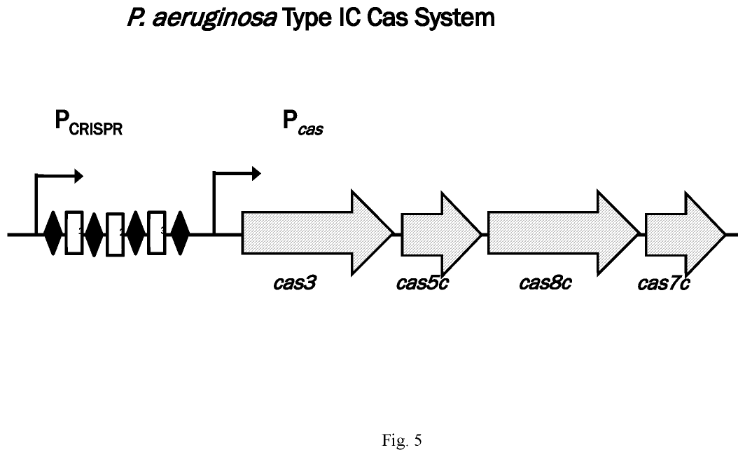

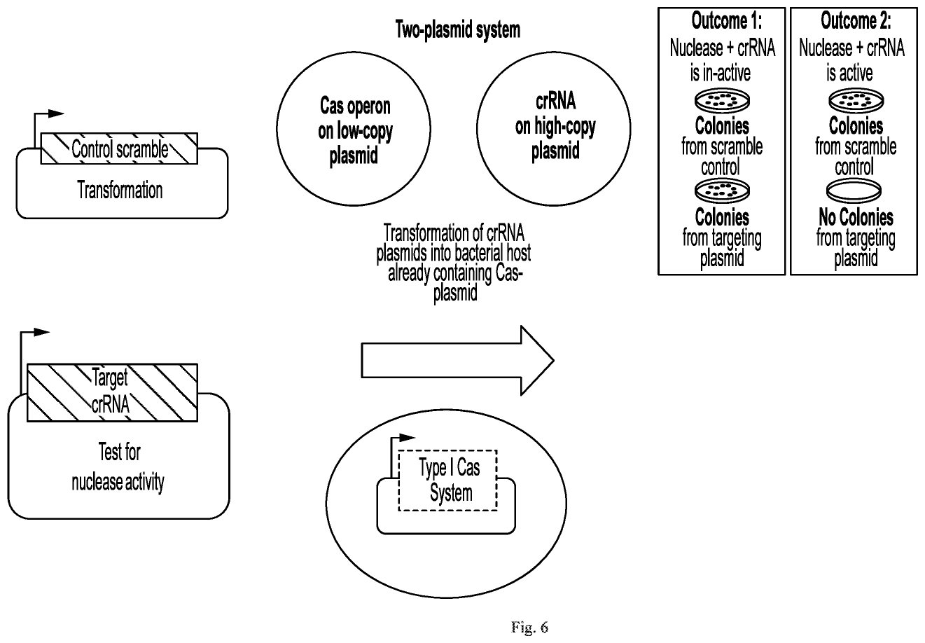

A two-plasmid system was used (illustrated in FIG. 6). One plasmid contained the CRISPR-Cas system expressed via a constitutive promoter (same promoter and RBS as in engineered phage) and a kanamycin resistance marker for selection. This plasmid was transformed into E. coli.

A second plasmid contained the individual crRNA expressed via a constitutive promoter (same promoter as in engineered phage) and an ampicillin resistance marker for selection. Electrocompetent cells were prepared from the E. coli strain harboring the CRISPR-Cas plasmid. The crRNA containing plasmid was then transformed into this strain.

Next, the killing efficacy of each tested spacer is determined. The plasmids listed in Table 5 are normalized to the same molar concentration. Each plasmid is transferred to the organism of interest by transformation, conjugation, or any other method for introducing a plasmid into a cell. The transformed cells are plated onto the appropriate selective media (e.g. antibiotic-containing agar). Following cell growth into colonies, the colonies that resulted from each different plasmid transfer are enumerated. Plasmids containing targeting spacers with a significantly lower transfer rate than the control plasmid containing the non-targeting spacer are considered to be successful at targeting the bacterial genome.

TABLE 5
Plasmids and controls used

| Plasmid | Function |
|---|---|
| Empty backbone vector | control for plasmid transfer efficiency |
| Vector containing the Cas system | control for Cas system toxicity |
| Vector containing the Cas system | control for off-target effects |
| and the non-targeting spacer | |
| Vector containing the Cas system | test sample |
| and the targeting spacer | |

### Example 4: Overview of E. coli Engineering Workflow

FIG. 5 shows an exemplary CRISPR-Cas system, the P. aeruginosa Type 1C Cas System. The PAIC gene operon is nearly 5700 bp, comprised of 4 separate proteins. Array of crRNA is expressed separately.

A plasmid-based assay was utilized to test efficacy of crRNA (illustrated in FIG. 6). A two-plasmid system was used. One plasmid contained the CRISPR-Cas system expressed via a constitutive promoter (same promoter and RBS as in engineered phage) and a kanamycin resistance marker for selection. This plasmid was transformed into E. coli.

A second plasmid contained the individual crRNA expressed via a constitutive promoter (same promoter as in engineered phage) and an ampicillin resistance marker for selection. Electrocompetent cells were prepared from the E. coli strain harboring the CRISPR-Cas plasmid. The crRNA containing plasmid was then transformed into this strain.

The number of colony forming units (CFU) formed after the transformation of either an active crRNA or a “scramble” control (randomized nucleotide sequence of same length as normal crRNA) were compared. The fewer colonies that developed, the greater the reduction attributable to the combination of the crRNA plasmid and the CRISPR-Cas plasmid.

For example, if the control crRNA plasmid transformation led to 1E7 CFUs and an active crRNA plasmid transformation led to 1E4 CFUs, that would represent a 3-log reduction attributed to CRISPR-Cas induced bacterial killing

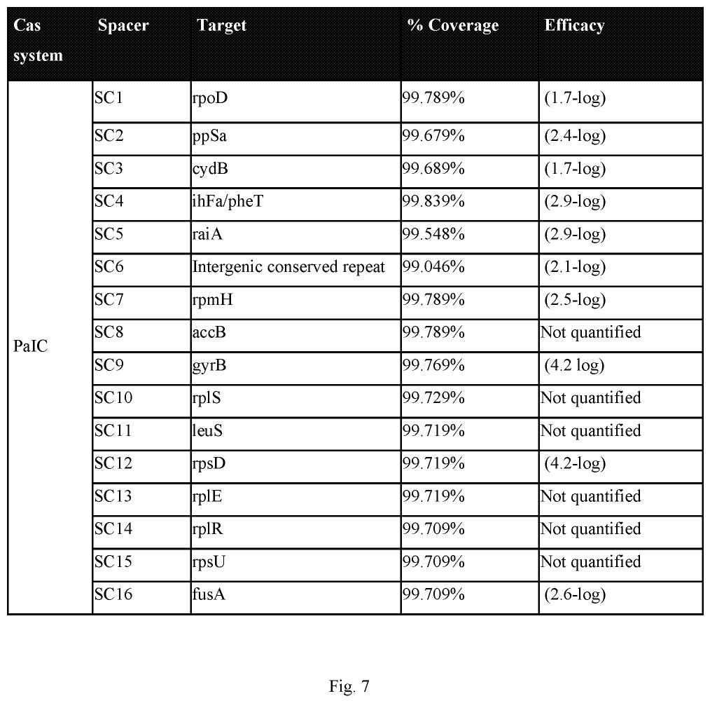

As shown in FIG. 7, the nominated spacers have high coverage and efficacy. The initial list of crRNA were chosen based on how conserved their target sequence was on a panel of 10,000 E. coli genomes publicly available. The efficacy column was determined based on the previously described plasmid-based assay. Highlighted spacer targets are the ones chosen for array testing. Selection was based on multiple parameters, including but not limited to percent coverage, efficacy and target among others.

### Example 5. Testing Efficacy of crRNA Array

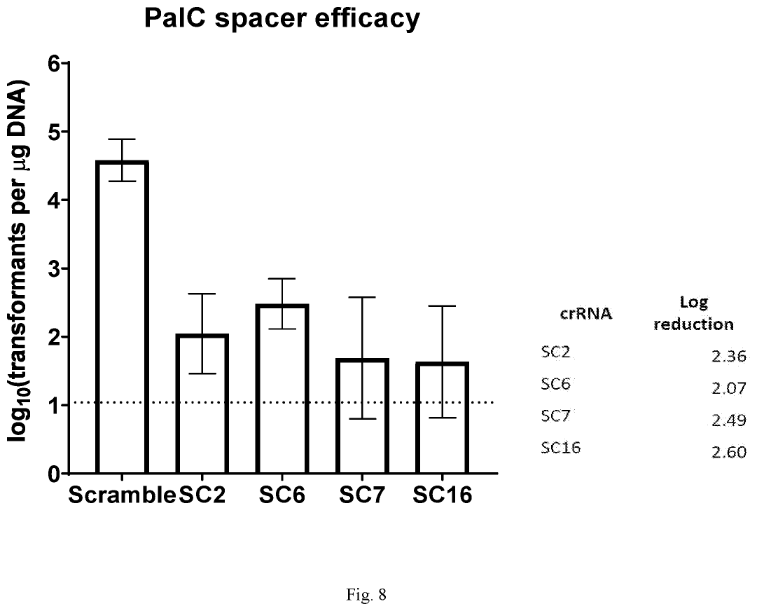

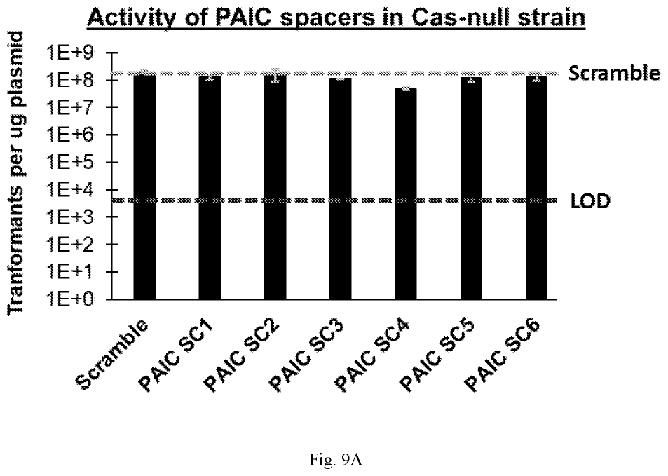

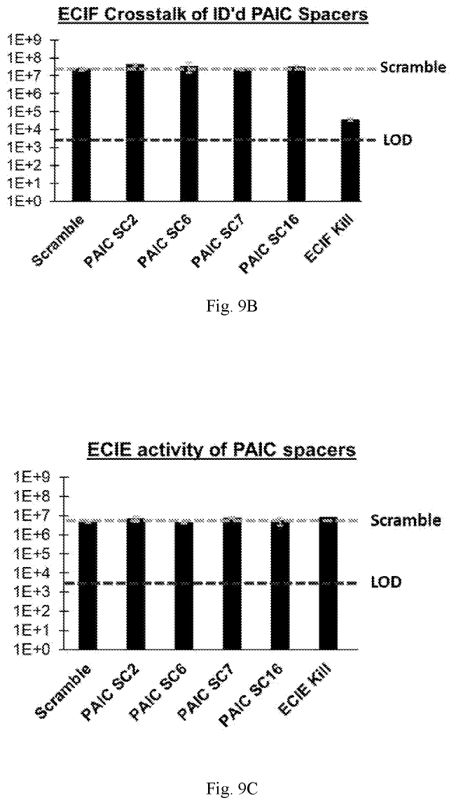

The spacer arrays were tested using the methods described above. FIG. 8 shows exemplary representative PaIC spacer efficiency, showing plasmid-based transformation results for 4 selected crRNA. Compared to scramble control, all four showed an approximately 2 log decreased in E. coli colonies. Replicates with no colonies are imputed to be ½ of limit of detection (LOD). Data was plotted as mean±S.D. The reduction in CFUs was not explained by general plasmid toxicity or another mechanism (i.e., antisense repression) as substantial CFU reductions are not observed in a Cas-null strain of E. coli (no PAIC CRISPR-Cas plasmid) (FIG. 9A). Additionally, there was no measurable reduction in CFU formation when the selected crRNA plasmids are transformed into E. coli strains possessing native CRISPR-Cas systems. A crRNA designed for the E. coli type IF (EcIF) system led to a multiple log reduction in CFUs, which indicate high specificity of the crRNA design, and absence of cross-talk of the identified PaIC spacers (FIG. 9B). A crRNA designed for the E. coli type IE (EcIE) did not activate the endogenous Cas system due to the absence of LeuO transcriptional regulator required to activate the native EcIE systems (FIG. 9C). K12 E. coli strain DH5-alpha strain was used for EcIE testing and E. coli NC101 strain was used for EcIF testing in this assay.

### Example 6. A Prediction Model for Number of Sites to be Targeted that can Sufficiently Avoid Mutational Escape

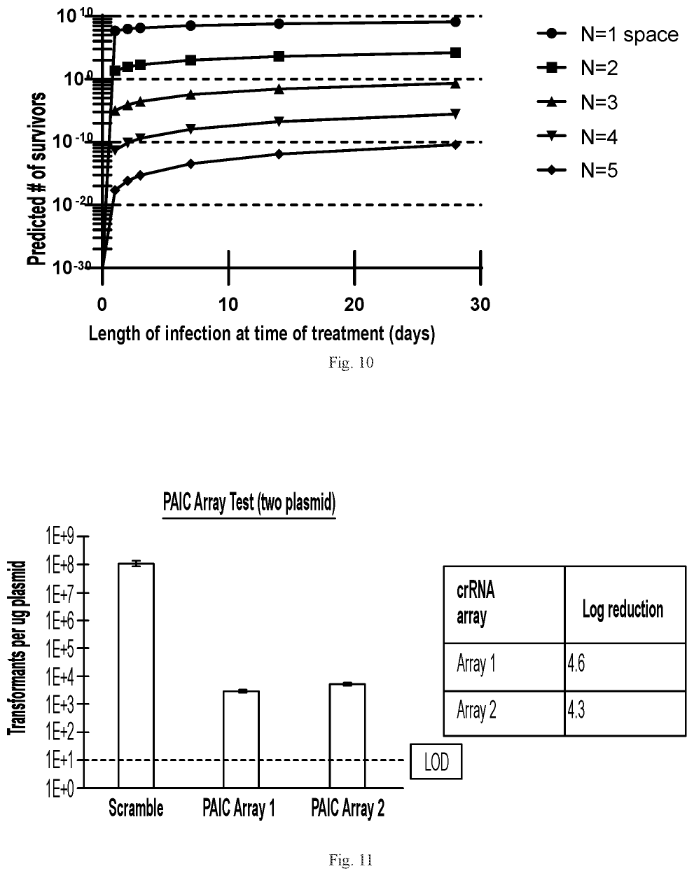

A basic model of mutation frequency was developed to understand the number of independent CRISPR-Cas targets needed to prevent escape. Data shown in FIG. 10 suggests that targeting 3 independent sites with CRISPR-Cas is sufficient to mitigate risk of mutational escape. This model assumes several worst-case parameters for example: (a) Normal mutation rate of 1e-6 per genome; (b) High bioburden of 10¹⁴ CFU (total GI microbiome or complete coating of ˜100 m² lung surface area); (c) High growth rate in vivo (20 minute doubling time); (d) Any mutation completely eliminates CRISPR-Cas activity. Under these highly stringent assumptions, 3 spacers are sufficient to completely suppress the emergence of resistance for a month of continuous infection, or ˜2,000 bacterial generations at 10¹⁴ CFU.

### Example 7. Analysis of PaIC Arrays 1 and 2

Based on the assays described in the previous sections, at least two arrays were further analyzed, PaIC array 1 and PaIC array 2. Table 9 characterizes the arrays.

TABLE 9
PaIC array 1 and PaIC array 2

| | |
|---|---|
| PaIC array 1 | PaIC array 2 |

| | Spacer ID | Coverage | Spacer ID | Coverage |
|---|---|---|---|---|
| Spacer 1 | SC7 | 99.789% | SC7 | 99.789% |
| Spacer 2 | SC16 | 99.709% | SC6 | 99.046% |
| Spacer 3 | SC2 | 99.679% | SC2 | 99.679% |

| | | |
|---|---|---|
| Total | 99.96% | 99.99% |

As indicated in Table 9, both the arrays have high overall coverage on about 10,000 clinical EC strains. A comparison of the array kill data using the plasmid based kill method described earlier, both PaIC arrays showed high competence in bacterial killing (FIG. 11). The array sequences are provided in Table 10 below.

TABLE 10
Nucleotide sequence of PaIC array 1 and PaIC array 2. Sequence includes
promoter,CRISPR repeats and three crRNA spacer sequences.

| Array | SEQ ID | SEQUENCE |
|---|---|---|
| PAIC | 45 | GAAAATTATTTTAAATTTCCTCTAGTCAGGCCGGAATAACTCCCTATAAT |
| array | | GCGACACCAGTCGCGCCCCGCACGGGCGCGTGGATTGAAACATTTATCA |
| 1 | | CAAAAGGATTGTTCGATGTCCAACAAGTCGCGCCCCGCACGGGCGCGTG |
| | | GATTGAAACAACCGTCTGTACTGAAGCGCAACCGTTCTCACGGGTCGCG |
| | | CCCCGCACGGGCGCGTGGATTGAAACTGAACAACGAAATCATCCTGGTA |
| | | ACCTGTGGTTCGTCGCGCCCCGCACGGGCGCGTGGATTGAAAC |
| PAIC | 43 | GAAAATTATTTTAAATTTCCTCTAGTCAGGCCGGAATAACTCCCTATAAT |
| array | | GCGACACCAGTCGCGCCCCGCACGGGCGCGTGGATTGAAACATTTATCA |
| 2 | | CAAAAGGATTGTTCGATGTCCAACAAGTCGCGCCCCGCACGGGCGCGTG |
| | | GATTGAAACAACCGTCTGTACTGAAGCGCAACCGTTCTCACGGGTCGCG |
| | | CCCCGCACGGGCGCGTGGATTGAAACATGCCGGATGCGGCGTGAACGCC |
| | | TTATCCGGCCTGTCGCGCCCCGCACGGGCGCGTGGATTGAAAC |

### Example 8: Purified and Cocktail E. coli Phage Host Range Assay

Data for purified and cocktail E. coli phage was acquired, reported in the best result from combined liquid and plaquing host range assays. The data summarized was the median of the binary hits across both liquid and plaquing host range for a given phage plus strain combination.

Determining liquid host range involved the addition of 5 uL of frozen, OD-controlled, culture material, 5 uL of known titer phage material and 40 uL of growth media into a well of a 364-well plate along with appropriate culture, phage, and media only controls. The plate was incubated for 20 hours at 37 C while shaking and OD600 readings were taken by the liquid handler every hour. The results were calculated by determining the ratio between areas under the cover (AUC) for samples with phage added and their respective controls. Samples with AUC ratios below 0.65 were considered positive (+) hits while AUC ratios greater than or equal to 0.65 were negative (−) hits.

For plaquing host range assays, bacterial strains of interest were cultured and screened for prophage. Bacteriophage of interest were serially diluted 50-fold across a microtiter plate from undiluted to 50-3 in 1×PBS. Agar overlays of strains used as titer host were poured and allowed to sit overnight. The following day, lysates for the bacterial strains of interest were spotted. After 15-20 min, the plates were imaged using the Hamilton-STAR-0062 and either counted by hand or run through an internally developed image analysis pipeline for transformation, background subtraction, and counting. Samples with a positive (+) number of plaque forming units were considered hits.

The results of this assay involving E. coli, wildtype Tequatrovirus phage (p004k and p00ex), wildtype Mosigvirus phage (p00c0), wildtype Phapecoctavirus phage (p00jc), wildtype Unique Myoviridae phage (p00ke), wildtype Vectrevirus phage (p5516), and cocktail CK507 were listed in Table 11. Cocktail CK507 comprises p004ke009, p00c0e030, p00ex014, p00jc, p00ke, and p5516. Overall, host range data was increased for cocktail CK507 as compared to purified phage.

TABLE 11
E. coli Phage Host Range for Wild-Type Phage and Cocktail CK507

| | Individual Phage | | | | | | Cocktail |
|---|---|---|---|---|---|---|---|
| Target | p004k | p00c0 | p00ex | p00jc | p00ke | p5516 | CK507 |
| b001024 | + | + | − | + | + | − | + |
| b001963 | − | − | − | − | − | − | + |
| b001965 | − | − | − | − | − | − | + |
| b001966 | − | − | + | + | + | − | + |
| b001991 | + | − | + | − | − | + | + |
| b001995 | − | − | − | − | − | − | + |
| b001997 | − | − | − | − | − | + | + |
| b001999 | − | − | + | + | + | − | + |
| b002027 | − | − | + | − | − | − | + |
| b002029 | − | − | − | − | − | + | + |
| b002030 | − | − | − | − | − | − | + |
| b002033 | + | − | − | − | − | − | + |
| b002034 | − | − | − | − | + | − | + |
| b002039 | − | − | + | − | − | − | + |
| b002073 | − | − | + | − | − | + | + |
| b002078 | − | − | − | − | − | − | + |
| b002105 | − | − | + | − | + | + | + |
| b002194 | + | − | + | − | − | + | + |
| b002205 | − | − | − | + | + | − | + |
| b002207 | − | − | + | − | − | − | + |
| b002208 | + | − | − | − | − | − | + |
| b002209 | + | − | − | − | − | − | + |
| b002232 | − | − | + | + | + | − | + |
| b002239 | − | − | − | + | + | + | + |
| b002245 | − | − | − | − | − | + | + |
| b002252 | − | − | − | + | + | − | + |
| b002257 | − | − | − | − | − | + | + |
| b002263 | − | − | − | + | − | − | + |
| b002301 | − | − | − | − | + | − | + |
| b002305 | − | − | − | + | − | − | + |
| b002308 | − | − | + | + | + | − | + |
| b002502 | − | − | + | − | − | + | + |
| b002503 | + | − | + | − | − | − | + |
| b002504 | − | − | + | + | − | − | + |
| b002505 | − | + | + | − | − | − | + |
| b002506 | − | − | + | − | − | − | + |
| b002507 | − | − | − | − | − | − | − |
| b002508 | − | − | − | − | − | − | + |
| b002509 | − | − | + | − | − | − | + |
| b002510 | − | − | − | − | − | − | + |
| b002511 | + | − | + | + | − | − | + |
| b002512 | − | − | + | − | − | − | + |
| b002513 | + | − | + | − | − | − | + |
| b002514 | − | − | − | − | − | − | − |
| b002515 | − | − | + | + | + | − | + |
| b002516 | + | − | + | − | − | − | + |
| b002517 | − | − | + | − | − | − | + |
| b002518 | − | − | + | − | − | + | + |
| b002519 | − | − | + | − | − | + | + |
| b002520 | + | − | − | − | − | − | + |
| b002521 | − | − | − | + | + | − | + |
| b002522 | + | − | + | − | − | − | + |
| b003247 | − | − | + | − | + | − | + |
| b003347 | − | − | − | − | − | − | + |
| b003348 | − | − | + | + | + | − | + |
| b003349 | − | + | + | + | + | − | + |
| b003350 | − | − | − | − | − | − | − |
| b003351 | − | − | − | − | − | − | − |
| b003352 | − | − | − | − | − | − | − |
| b003353 | − | − | + | − | − | − | + |
| b003354 | − | − | − | + | + | − | + |
| b003355 | − | − | + | + | − | − | + |
| b003356 | − | − | − | + | − | − | + |
| b003357 | − | − | + | + | + | − | + |
| b003358 | − | − | − | − | − | − | + |
| b003359 | − | − | − | − | − | − | − |
| b003360 | − | − | − | − | − | − | + |
| b003361 | + | − | + | + | − | − | + |
| b003362 | − | − | + | + | − | − | + |
| b003363 | + | − | − | − | − | − | + |
| b003364 | − | − | + | − | + | − | + |
| b003365 | − | − | − | − | − | − | − |
| b003366 | − | − | + | − | + | − | + |
| b003367 | − | − | − | − | − | − | − |
| b003368 | − | − | + | − | − | + | + |
| b003369 | + | − | + | + | − | − | + |
| b003370 | − | − | + | + | + | − | + |
| b003371 | − | − | − | + | + | − | + |
| b003372 | − | − | − | − | − | − | − |
| b003373 | − | − | − | − | − | − | − |
| b003374 | + | − | + | − | + | − | + |
| b003375 | − | − | + | − | − | + | + |
| b003376 | − | − | + | − | − | + | + |
| b003377 | − | − | + | − | − | − | + |
| b003378 | − | − | − | + | − | − | + |
| b003379 | − | − | + | − | − | + | + |
| b003380 | − | − | − | − | − | − | − |
| b003381 | + | − | + | − | − | − | + |
| b003382 | − | − | + | − | − | − | + |
| b003383 | − | − | − | + | + | − | + |
| b003384 | + | − | + | + | − | − | + |
| b003385 | − | − | + | − | − | + | + |
| b003386 | − | − | − | + | + | + | + |
| b003387 | + | − | + | + | + | − | + |
| b003388 | + | − | + | − | − | − | + |
| b003389 | − | − | + | − | − | − | + |
| b003390 | − | − | + | − | − | + | + |
| b003391 | + | − | + | + | − | − | + |
| b003392 | − | − | + | − | − | − | + |
| b003393 | − | − | − | − | − | − | + |
| b003394 | + | − | + | − | − | + | + |
| b003395 | + | − | + | + | + | − | + |
| b003396 | + | − | + | − | − | − | + |
| b003397 | + | − | + | − | − | − | + |
| b003398 | − | − | − | − | + | − | + |
| b003399 | − | − | − | − | − | − | + |
| b003400 | + | − | + | − | − | − | + |
| b003401 | + | − | + | − | − | − | + |
| b003402 | + | − | + | − | − | − | + |
| b003403 | − | − | − | + | + | − | + |
| b003404 | − | − | + | − | − | − | + |
| b003405 | − | − | + | + | + | − | + |
| b003406 | − | + | + | − | − | − | + |
| b003407 | + | − | + | − | − | + | + |
| b003408 | − | − | + | − | − | + | + |
| b003409 | + | − | + | + | + | − | + |
| b003410 | − | − | + | − | − | − | + |
| b003411 | + | − | + | + | + | − | + |
| b003412 | + | − | + | − | − | − | + |
| b003413 | − | − | − | − | − | + | + |
| b003414 | − | − | + | + | + | + | + |
| b003415 | − | − | − | − | − | − | + |
| b003416 | − | − | − | − | − | − | + |
| b003417 | + | − | + | + | + | + | + |
| b003418 | + | − | + | − | − | + | + |
| b003419 | + | − | − | − | − | − | + |
| b003420 | − | − | + | + | + | + | + |
| b003421 | − | − | − | − | − | − | + |
| b003422 | + | + | − | + | + | − | + |
| b003675 | − | − | + | − | − | + | + |
| b003762 | − | − | + | − | − | + | + |
| b003763 | − | − | − | − | − | − | + |
| b003764 | − | − | − | − | − | − | + |
| b003765 | + | − | − | − | − | − | + |
| b003770 | + | − | + | + | + | − | + |
| b003771 | − | − | − | + | − | − | + |
| b003773 | − | − | + | − | − | + | + |
| b003774 | + | − | + | − | − | − | + |
| b003777 | − | − | − | − | + | − | + |
| b003779 | − | − | − | − | − | − | + |
| b003780 | − | − | − | − | + | − | + |
| b003781 | − | − | − | − | − | − | − |
| b003782 | + | − | + | + | + | − | + |
| b003783 | − | − | + | + | + | − | + |
| b003786 | − | − | − | − | − | − | + |
| b003787 | − | − | + | − | − | − | + |
| b003788 | − | − | + | − | − | + | + |
| b003790 | − | − | + | − | − | − | + |
| b003791 | − | − | − | + | + | − | + |
| b003793 | − | − | + | − | − | + | + |
| b003795 | − | − | − | − | − | − | + |
| b003798 | − | − | + | − | − | + | + |
| b003802 | − | − | − | − | − | − | − |
| b003804 | − | − | − | − | − | − | + |
| b003805 | − | − | + | + | − | − | + |
| b003806 | − | − | − | − | − | − | + |
| b003812 | + | − | + | − | − | − | + |
| b003813 | + | − | + | − | + | − | + |
| b003815 | − | − | + | − | + | − | + |
| b003818 | + | − | − | + | + | − | + |
| b003821 | − | − | + | + | + | − | + |
| b003823 | + | − | + | + | + | − | + |
| b003826 | − | − | − | − | − | − | − |
| b003827 | − | − | − | − | − | − | + |
| b003829 | − | − | − | − | − | − | − |
| b003830 | − | − | + | − | − | + | + |
| b003838 | − | − | − | + | − | − | + |
| b003840 | − | − | − | − | − | − | − |
| b003841 | − | − | + | − | − | − | + |
| b003843 | − | − | − | − | − | − | − |
| b003845 | + | − | + | + | + | − | + |
| b003850 | − | − | − | − | − | − | − |
| b003851 | − | − | + | − | + | + | + |
| b003852 | + | − | + | + | + | − | + |
| b003853 | + | − | + | − | − | + | + |
| b003855 | + | − | + | − | − | − | + |
| b003856 | − | − | + | − | − | + | + |
| b003864 | + | − | + | − | − | − | + |
| b003865 | + | + | + | + | − | − | + |
| b003868 | + | − | − | + | + | − | + |
| b003871 | − | − | + | + | + | − | + |
| b003872 | − | − | + | − | − | − | + |
| b003874 | − | − | − | − | − | − | − |
| b003880 | − | − | − | + | − | − | + |
| b003885 | − | − | − | − | − | − | + |
| b003886 | − | − | − | − | − | − | + |
| b003887 | − | − | − | + | + | − | + |
| b003889 | + | − | + | + | − | − | + |
| b003890 | + | − | + | − | − | − | + |
| b003893 | − | − | − | + | + | − | + |
| b003894 | − | − | − | − | − | + | + |
| b003896 | − | − | − | − | − | − | + |
| b003897 | − | − | − | − | − | − | − |
| b003898 | − | − | + | − | − | + | + |
| b003899 | − | − | + | − | − | + | + |
| b003900 | − | − | + | − | − | − | + |
| b003902 | + | − | + | + | + | − | + |
| b003903 | − | − | + | − | − | + | + |
| b003907 | − | − | − | − | + | + | + |
| b003908 | − | − | − | + | + | + | + |
| b003909 | − | − | − | + | + | − | + |
| b003912 | − | − | + | + | + | + | + |
| b003920 | + | − | + | − | + | − | + |
| b003922 | − | − | − | − | − | + | + |
| b003923 | − | − | + | − | − | − | + |
| b003924 | − | − | + | − | − | + | + |
| b003926 | + | − | + | + | + | − | + |
| b003930 | − | − | + | − | − | + | + |
| b003931 | − | − | + | − | − | + | + |
| b003932 | − | − | + | − | + | + | + |
| b003934 | + | − | + | − | + | − | + |
| b003935 | − | − | − | − | − | − | − |
| b003941 | − | − | − | − | − | + | + |
| b003942 | − | − | − | − | − | − | + |
| b003943 | − | − | − | − | − | − | − |
| b003944 | − | − | − | − | + | − | + |
| b003945 | − | − | + | − | − | − | + |
| b003947 | − | − | − | − | − | − | + |
| b003948 | − | − | − | − | − | − | − |
| b003949 | − | − | − | + | − | − | + |
| b003952 | − | − | + | − | + | − | + |
| b003954 | − | − | − | − | − | − | − |
| b003956 | − | − | − | + | + | − | + |
| b003957 | − | − | + | + | + | − | + |
| b003958 | − | − | − | − | − | − | + |
| b003962 | + | − | + | − | − | − | + |
| b003963 | + | − | + | − | − | − | + |
| b003965 | − | − | − | − | − | − | − |
| b003969 | + | − | + | − | − | − | + |
| b003970 | − | − | + | − | − | + | + |
| b003974 | − | − | + | − | − | − | + |
| b003976 | − | − | − | − | − | − | + |
| b003977 | − | − | + | − | − | − | + |
| b003978 | + | − | − | + | + | − | + |
| b003979 | + | − | + | + | + | − | + |
| b003982 | + | − | + | − | − | − | + |
| b003983 | + | − | − | − | − | − | + |
| b003984 | − | − | + | + | + | − | + |
| b003986 | − | − | + | + | + | − | + |
| b003989 | − | − | − | + | − | − | + |
| b003990 | − | − | − | − | + | − | + |
| b003996 | − | − | − | − | − | − | − |
| b003998 | + | − | + | + | + | + | + |
| b004000 | − | − | + | − | − | + | + |
| b004008 | − | − | − | − | − | − | − |
| b004009 | − | − | + | + | − | − | + |
| b004012 | − | − | + | − | − | − | + |
| b004013 | − | − | − | − | − | − | + |
| b004014 | + | − | + | − | − | − | + |
| b004015 | − | − | + | − | − | + | + |
| b004016 | − | − | + | − | − | + | + |
| b004017 | − | − | − | + | + | − | + |
| b004018 | + | − | + | − | − | − | + |
| b004020 | − | − | − | + | − | − | + |
| b004021 | − | − | + | − | − | − | + |
| b004022 | − | − | − | − | − | − | − |
| b004023 | − | − | + | − | − | − | + |
| b004025 | − | − | − | − | − | − | − |
| b004026 | − | − | − | − | + | − | + |
| b004027 | − | − | + | + | + | − | + |
| b004028 | − | − | + | − | − | + | + |
| b004031 | + | − | + | − | − | − | + |
| b004032 | − | − | − | − | − | − | − |
| b004033 | − | − | − | − | − | − | + |
| b004034 | − | − | − | − | + | − | + |
| b004037 | − | − | − | + | − | − | + |
| b004038 | − | − | − | − | − | − | − |
| b004039 | − | − | − | − | − | − | + |
| b004040 | − | − | − | + | + | − | + |
| b004041 | − | − | − | − | − | − | + |
| b004043 | − | − | − | + | − | − | − |
| b004044 | − | − | − | + | + | − | + |
| b004045 | − | − | + | − | − | − | + |
| b004046 | + | + | + | + | + | − | + |
| b004048 | + | − | + | − | − | + | + |
| b004049 | − | − | − | − | − | − | + |
| b004052 | − | − | − | + | + | − | + |
| b004057 | − | − | + | − | − | − | + |
| b004058 | − | − | + | + | + | − | + |
| b004059 | − | − | − | + | + | − | + |
| b004060 | − | − | + | − | + | + | + |
| b004092 | − | − | + | − | − | + | + |
| b004096 | + | − | − | − | − | − | + |
| b004097 | − | − | − | − | − | − | − |
| b004098 | + | − | + | − | − | − | + |
| b004099 | − | − | − | − | + | − | + |
| b004100 | − | − | − | − | − | − | + |
| b004101 | − | − | + | − | − | − | + |
| b004102 | − | − | − | − | − | − | − |
| b004103 | − | − | − | − | − | − | − |
| b004104 | − | − | − | − | − | − | − |
| b004105 | − | − | + | − | − | + | + |
| b004106 | − | − | + | − | − | − | + |
| b004107 | − | − | − | − | − | − | − |
| b004108 | − | − | − | − | − | − | − |
| b004109 | − | − | − | − | − | − | − |
| b004110 | − | − | − | − | − | − | + |
| b004111 | − | − | − | − | − | − | + |
| b004112 | − | − | − | + | − | − | + |
| b004113 | − | − | − | − | − | − | − |
| b004114 | − | − | + | + | + | − | + |
| b004115 | − | − | − | + | + | − | + |
| b004116 | − | − | − | − | − | − | − |
| b004117 | − | − | + | − | − | + | + |

### Example 9: Full Construct in a Second E. coli Phage Assay

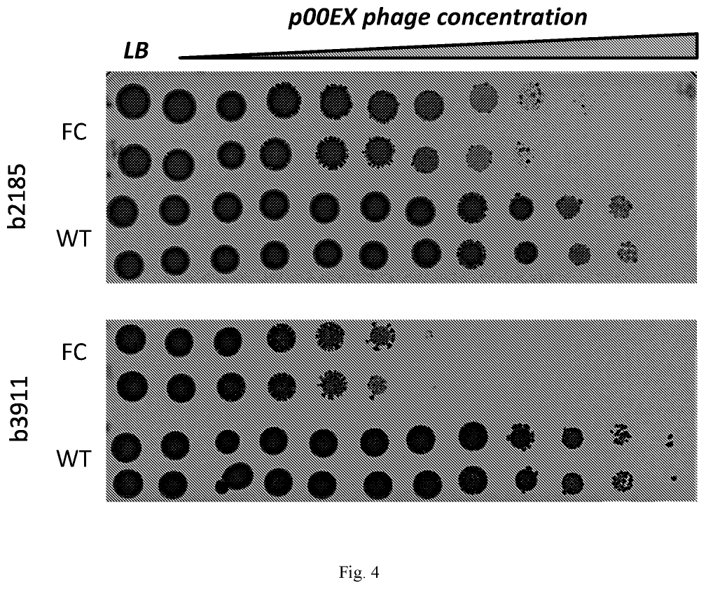

A standard plate kill assay starting at 3e10 PFU/ml (MOI of ˜100) and serial 1:5 dilutions of phage with the same bacterial concentration was completed after the E. coli bacteria (strains b2185 and b3911) and phage were mixed and immediately spotted onto a plate. p00Ex wildtype and engineered phage were mixed with bacteria in logarithmic growth and plated immediately in 2 ul spots on LB agar. The ratio of phage to bacteria was altered through the dilution series so that the amount of bacteria stayed constant at each dilution but the amount of phage was a 1 to 5 dilution. At the highest dilution, the multiplicity of infection (MOI) was 100, meaning there were approximately 100 phages per bacteria. In FIG. 4, p00EXe014 (FC) killed both the b2185 and b3911 strains consistently better than the p00EX wildtype (WT) counterpart. The data for both E. coli strains, validated the full construct phage was working as expected, validating the standard plate kill assay.

### Example 10. Purified Phage Cocktail CK618

A purified phage cocktail including six phages (p004k, p00jc, p00c0, p00ke, p5516 and p00ex) are described. Both potency and purity are evaluated for consistency with clinical trial material including endotoxin levels.

TABLE 12
Components of cocktail

| Phage | Genus | Construct incorporated |
|---|---|---|
| p00exe014 | Tequatrovirus | Full Construct |
| p004Ke009 | Tequatrovirus | Full Construct |
| p00jc | Phapecoctavirus | Wild Type |
| p00ke | Unclassified Myovirus | Wild Type |
| p00c0e030 | Mosigvirus | Full Construct |
| p5516 | Vectrevirus | Wild Type |

Table 13 shows E. coli Phange Host Range in Cocktail CK618

| | Individual Phage | | | | | | Cocktail |
|---|---|---|---|---|---|---|---|
| Target | p004Kexe014 | p00c0e030 | p00exe014 | p00jc | p00ke | p5516 | CK618 |
| b001024 | + | + | + | + | + | + | + |
| b001963 | − | − | + | − | + | + | + |
| b001965 | + | + | + | − | − | − | + |
| b001966 | + | + | + | + | + | + | + |
| b001991 | + | − | + | − | + | + | + |
| b001995 | − | + | + | + | − | − | + |
| b001997 | + | + | + | − | + | + | + |
| b001999 | + | + | + | + | − | + | + |
| b002027 | + | + | + | + | − | − | + |
| b002029 | − | + | + | − | + | + | + |
| b002030 | − | + | + | + | + | − | + |
| b002033 | + | + | + | + | − | − | + |
| b002034 | + | + | + | + | + | + | + |
| b002039 | + | + | + | − | + | + | + |
| b002073 | + | − | + | − | + | + | + |
| b002078 | − | + | + | − | − | − | + |
| b002105 | − | + | + | − | + | + | + |
| b002194 | + | − | + | − | + | + | + |
| b002205 | + | + | + | + | + | + | + |
| b002207 | + | + | + | + | + | + | + |
| b002208 | + | − | + | − | − | + | + |
| b002209 | + | + | + | − | + | + | + |
| b002232 | − | + | + | + | − | − | + |
| b002239 | − | − | + | + | + | + | + |
| b002245 | − | + | + | − | + | + | + |
| b002252 | − | + | + | + | + | − | + |
| b002257 | − | + | + | − | + | + | + |
| b002263 | − | + | + | + | + | + | + |
| b002301 | + | + | + | + | + | + | + |
| b002305 | − | + | + | + | − | − | + |
| b002308 | + | + | + | + | − | − | + |
| b002502 | + | + | + | − | + | + | + |
| b002503 | + | + | + | − | + | + | + |
| b002504 | − | + | + | + | − | − | + |
| b002505 | − | + | + | − | − | − | + |
| b002506 | − | + | + | + | − | − | + |
| b002507 | − | − | − | + | + | + | + |
| b002508 | + | + | + | + | + | + | + |
| b002509 | + | + | + | − | + | − | + |
| b002510 | − | − | − | − | − | − | − |
| b002511 | + | + | + | + | − | − | + |
| b002512 | − | + | + | − | − | − | + |
| b002513 | + | − | + | − | + | + | + |
| b002514 | − | + | + | − | − | − | + |
| b002515 | + | + | + | + | − | − | + |
| b002516 | + | − | + | − | + | + | + |
| b002517 | + | − | + | + | + | + | + |
| b002518 | + | + | + | − | + | + | + |
| b002519 | − | + | + | − | + | + | + |
| b002520 | + | − | + | − | − | − | + |
| b002521 | − | − | − | + | + | + | + |
| b002522 | + | + | + | + | − | + | + |
| b003247 | + | + | + | − | + | − | + |
| b003347 | − | + | + | − | + | + | + |
| b003348 | + | + | + | + | + | − | + |
| b003349 | + | + | + | + | − | − | + |
| b003350 | + | + | + | + | + | + | + |
| b003351 | − | + | + | − | − | − | + |
| b003352 | − | + | + | − | − | − | + |
| b003353 | − | + | + | − | − | − | + |
| b003354 | + | + | + | + | + | + | + |
| b003355 | − | + | + | + | − | − | + |
| b003356 | + | − | + | + | + | − | + |
| b003357 | + | + | + | + | + | − | + |
| b003358 | − | + | − | − | − | − | + |
| b003359 | − | + | − | + | + | − | + |
| b003360 | + | − | + | − | − | − | + |
| b003361 | + | + | + | + | + | + | + |
| b003362 | − | − | + | + | − | + | + |
| b003363 | + | + | + | + | + | + | + |
| b003364 | − | + | + | − | − | + | + |
| b003365 | − | − | − | − | − | − | − |
| b003366 | + | − | + | − | + | + | + |
| b003367 | − | + | − | + | − | − | + |
| b003368 | − | + | + | − | + | + | + |
| b003369 | + | − | + | + | + | + | + |
| b003370 | + | + | + | + | + | + | + |
| b003371 | + | + | + | + | + | + | + |
| b003372 | + | + | − | + | + | − | + |
| b003373 | − | + | + | − | − | − | + |
| b003374 | + | − | + | − | + | + | + |
| b003375 | + | − | + | + | + | + | + |
| b003376 | − | + | + | − | + | + | + |
| b003377 | + | − | + | − | − | + | + |
| b003378 | − | + | + | + | + | − | + |
| b003379 | − | + | + | − | + | + | + |
| b003380 | − | + | + | − | − | − | + |
| b003381 | + | + | + | − | + | − | + |
| b003382 | − | + | + | − | − | − | + |
| b003383 | − | + | + | + | + | + | + |
| b003384 | + | + | + | + | − | + | + |
| b003385 | − | + | + | − | + | + | + |
| b003386 | + | + | + | + | + | − | + |
| b003387 | + | + | + | + | + | + | + |
| b003388 | + | − | + | − | + | + | + |
| b003389 | − | + | + | − | + | + | + |
| b003390 | − | + | + | − | + | + | + |
| b003391 | + | + | + | + | + | + | + |
| b003392 | + | − | + | − | − | + | + |
| b003393 | + | + | + | − | + | + | + |
| b003394 | + | + | + | + | + | + | + |
| b003395 | + | + | + | + | + | − | + |
| b003396 | + | + | + | − | − | − | + |
| b003397 | + | + | + | − | + | − | + |
| b003398 | + | − | + | + | + | + | + |
| b003399 | + | + | + | + | + | − | + |
| b003400 | + | + | + | + | + | + | + |
| b003401 | + | + | + | + | − | − | + |
| b003402 | + | + | + | − | − | − | + |
| b003403 | + | − | − | + | + | − | + |
| b003404 | − | + | + | − | − | − | + |
| b003405 | − | + | + | + | − | − | + |
| b003406 | + | + | + | − | − | − | + |
| b003407 | + | − | + | − | + | + | + |
| b003408 | + | + | + | + | + | − | + |
| b003409 | + | + | + | + | + | + | + |
| b003410 | − | + | + | + | + | − | + |
| b003411 | + | + | + | + | + | + | + |
| b003412 | + | + | + | − | + | − | + |
| b003413 | + | − | + | + | + | − | + |
| b003414 | + | + | + | + | − | + | + |
| b003415 | + | + | + | + | + | − | + |
| b003416 | − | + | + | − | − | − | + |
| b003417 | + | + | + | + | + | + | + |
| b003418 | + | + | + | − | + | + | + |
| b003419 | + | + | + | − | + | + | + |
| b003420 | − | + | + | + | + | − | + |
| b003421 | + | − | − | − | + | − | + |
| b003422 | + | + | + | + | + | + | + |
| b003675 | + | + | + | − | + | + | + |
| b003762 | − | + | + | − | + | + | + |
| b003763 | + | − | + | − | − | − | + |
| b003764 | + | + | − | + | − | − | + |
| b003765 | + | + | + | + | + | − | + |
| b003770 | + | + | + | + | + | + | + |
| b003771 | − | − | − | + | + | + | + |
| b003773 | − | + | + | − | + | + | + |
| b003774 | + | + | + | − | + | − | + |
| b003777 | − | + | − | + | + | + | + |
| b003779 | + | + | + | + | + | − | + |
| b003780 | − | + | + | − | − | − | + |
| b003781 | + | − | − | − | − | − | − |
| b003782 | + | + | + | + | + | + | + |
| b003783 | + | + | + | + | + | + | + |
| b003786 | + | − | − | + | − | + | + |
| b003787 | + | + | + | + | − | + | + |
| b003788 | + | + | + | + | + | + | + |
| b003790 | − | + | + | − | − | − | + |
| b003791 | + | + | + | + | − | + | + |
| b003793 | + | + | + | + | + | + | + |
| b003795 | − | − | + | + | − | − | + |
| b003798 | − | + | + | − | + | + | + |
| b003802 | − | − | − | − | − | − | − |
| b003804 | − | + | + | + | − | + | + |
| b003805 | − | + | + | + | + | + | + |
| b003806 | − | − | − | + | + | − | + |
| b003812 | + | + | + | + | + | − | + |
| b003813 | + | + | + | + | + | + | + |
| b003815 | + | + | + | + | + | + | + |
| b003818 | + | + | + | + | + | + | + |
| b003821 | − | + | + | + | + | − | + |
| b003823 | + | + | + | + | + | − | + |
| b003826 | − | − | − | − | − | − | − |
| b003827 | − | + | − | − | + | + | + |
| b003829 | − | − | − | − | − | − | − |
| b003830 | − | + | + | − | + | + | + |
| b003838 | − | − | + | + | − | − | + |
| b003840 | + | − | − | − | − | − | + |
| b003841 | + | + | + | − | − | + | + |
| b003843 | + | + | − | − | − | − | + |
| b003845 | + | + | + | + | + | + | + |
| b003850 | − | − | − | + | − | − | − |
| b003851 | − | + | + | − | + | + | + |
| b003852 | + | + | + | + | + | + | + |
| b003853 | + | + | + | + | + | + | + |
| b003855 | + | + | + | + | + | + | + |
| b003856 | − | + | + | − | + | + | + |
| b003864 | + | + | + | − | + | + | + |
| b003865 | + | + | + | + | + | − | + |
| b003868 | + | − | + | + | + | + | + |
| b003871 | + | + | + | + | + | + | + |
| b003872 | − | + | + | + | + | − | + |
| b003874 | − | − | − | + | − | + | − |
| b003880 | + | − | + | − | + | − | + |
| b003885 | + | + | + | + | − | − | + |
| b003886 | − | + | − | + | + | + | + |
| b003887 | + | + | + | + | + | − | + |
| b003889 | + | + | + | + | + | + | + |
| b003890 | + | − | + | − | − | + | + |
| b003893 | + | + | + | + | + | + | + |
| b003894 | − | + | + | − | + | + | + |
| b003896 | − | − | − | + | + | + | + |
| b003897 | − | − | − | − | − | − | − |
| b003898 | + | + | + | − | + | + | + |
| b003899 | − | + | + | − | + | + | + |
| b003900 | − | + | + | − | + | − | + |
| b003902 | + | + | + | + | + | + | + |
| b003903 | + | + | + | − | + | − | + |
| b003907 | − | + | + | − | + | + | + |
| b003908 | − | − | − | + | + | + | + |
| b003909 | − | + | + | + | + | + | + |
| b003912 | + | + | + | + | + | − | + |
| b003920 | + | + | + | − | + | + | + |
| b003922 | − | + | + | − | + | + | + |
| b003923 | + | + | + | − | + | + | + |
| b003924 | − | − | + | − | + | − | + |
| b003926 | + | + | + | + | + | + | + |
| b003930 | − | + | + | − | + | + | + |
| b003931 | + | + | + | − | + | + | + |
| b003932 | − | + | + | − | + | + | + |
| b003934 | + | + | + | − | + | + | + |
| b003935 | − | − | − | − | − | − | − |
| b003941 | − | + | + | − | + | + | + |
| b003942 | + | + | + | + | + | + | + |
| b003943 | − | − | − | + | + | − | + |
| b003944 | + | + | + | − | − | − | + |
| b003945 | − | + | + | − | + | + | + |
| b003947 | + | − | + | + | + | − | + |
| b003948 | − | − | − | − | − | − | − |
| b003949 | + | + | + | + | − | − | + |
| b003952 | + | + | + | − | + | + | + |
| b003954 | − | − | − | − | − | − | − |
| b003956 | + | + | + | + | − | − | + |
| b003957 | − | + | + | + | − | − | + |
| b003958 | + | + | + | + | − | − | + |
| b003962 | + | + | + | − | + | − | + |
| b003963 | + | + | + | − | + | − | + |
| b003965 | + | + | + | − | − | + | + |
| b003969 | + | − | + | − | + | + | + |
| b003970 | − | + | + | − | + | + | + |
| b003974 | − | + | + | − | + | + | + |
| b003976 | + | + | + | + | − | − | + |
| b003977 | + | − | + | + | + | − | + |
| b003978 | + | − | − | + | + | + | + |
| b003979 | + | + | + | + | + | + | + |
| b003982 | + | + | + | + | + | + | + |
| b003983 | + | − | − | − | + | + | + |
| b003984 | + | − | + | + | − | − | + |
| b003986 | − | + | + | + | + | + | + |
| b003989 | + | + | + | + | + | − | + |
| b003990 | − | − | + | − | + | + | + |
| b003996 | + | − | + | + | + | − | + |
| b003998 | + | + | + | + | + | + | + |
| b004000 | − | + | + | − | + | + | + |
| b004008 | − | − | − | − | − | − | − |
| b004009 | + | − | + | + | − | − | + |
| b004012 | − | + | + | − | + | + | + |
| b004013 | − | + | − | + | − | + | + |
| b004014 | + | + | + | − | + | + | + |
| b004015 | − | + | + | − | + | + | + |
| b004016 | − | + | + | − | + | + | + |
| b004017 | + | + | + | + | + | + | + |
| b004018 | + | + | + | − | + | − | + |
| b004020 | + | − | + | + | − | + | + |
| b004021 | + | + | + | − | − | − | + |
| b004022 | − | − | − | − | − | − | − |
| b004023 | − | + | + | − | + | + | + |
| b004025 | − | − | − | + | − | − | − |
| b004026 | + | + | + | + | − | + | + |
| b004027 | + | + | + | + | + | − | + |
| b004028 | − | + | + | − | + | + | + |
| b004031 | + | + | + | + | + | + | + |
| b004032 | − | − | − | − | − | − | − |
| b004033 | − | − | + | + | + | − | + |
| b004034 | − | − | − | + | + | + | + |
| b004037 | − | + | + | − | − | − | + |
| b004038 | − | − | − | + | − | − | + |
| b004039 | + | − | + | + | + | − | + |
| b004040 | − | + | − | + | + | + | + |
| b004041 | − | + | + | + | + | + | + |
| b004043 | − | − | − | + | + | − | + |
| b004044 | + | + | + | + | + | + | + |
| b004045 | + | − | + | − | + | + | + |
| b004046 | + | + | + | + | + | + | + |
| b004048 | + | + | + | − | + | + | + |
| b004049 | − | + | + | + | + | + | + |
| b004052 | + | + | + | + | + | − | + |
| b004057 | + | + | + | − | + | + | + |
| b004058 | + | + | + | − | − | − | + |
| b004059 | − | + | − | + | + | + | + |
| b004060 | − | + | + | − | + | + | + |
| b004092 | + | + | + | + | + | + | + |
| b004096 | + | + | + | + | + | − | + |
| b004097 | − | − | − | − | − | − | − |
| b004098 | + | + | + | + | + | − | + |
| b004099 | + | + | + | + | + | + | + |
| b004100 | + | + | − | + | + | − | + |
| b004101 | − | + | + | + | + | + | + |
| b004102 | − | − | − | + | + | − | + |
| b004103 | − | − | − | + | − | − | − |
| b004104 | − | − | − | + | − | − | − |
| b004105 | − | + | + | − | + | + | + |
| b004106 | + | + | + | + | − | − | + |
| b004107 | − | − | − | + | − | − | + |
| b004108 | − | − | − | − | − | − | − |
| b004109 | − | − | − | − | − | − | − |
| b004110 | + | − | + | + | + | − | + |
| b004111 | + | + | + | + | + | + | + |
| b004112 | − | − | − | + | + | + | + |
| b004113 | − | − | − | − | − | − | − |
| b004114 | − | + | + | + | − | − | + |
| b004115 | + | − | − | + | + | − | + |
| b004116 | − | − | − | + | + | − | + |
| b004117 | − | − | + | + | + | + | + |

### Example 11. High Titer Liquid and Plaquing Host Range for Cocktail CK618

The objective of this study was to determine the total host range of CK618 and its component individual phages by two distinct methods.

### Materials and Methods

A clinically relevant 300-isolate Clinical Panel (CP) was developed. Escherichia coli (E. coli) isolates were sourced from the International Health Management Associates (IHMA) and metadata regarding various characteristics of each strain was obtained. Strains were defined as MDR if they were resistant to three or more classes of antibiotics, summarized in Table 14.

TABLE 14
Host Range Panel Metadata
Regional distribution

| Region | # isolates | % of total panel¹ |
|---|---|---|
| North America | 127 | 42.3 |
| Europe | 86 | 29 |
| Latin America | 44 | 15 |
| Asia | 43 | 14 |

Country distribution

| Country | # isolates | % of total panel¹ |
|---|---|---|
| United States | 127 | 42.3 |
| Turkey | 21 | 7.0 |
| Argentina | 21 | 7.0 |
| Russia | 19 | 6.3 |
| Brazil | 18 | 6.0 |
| Japan | 11 | 3.7 |
| Ukraine | 10 | 3.3 |
| Hong Kong | 10 | 3.3 |
| Taiwan | 9 | 3 |
| Italy | 9 | 3 |
| Spain | 8 | 3 |
| Thailand | 8 | 3 |
| Germany | 7 | 2 |
| France | 6 | 2 |
| United Kingdom | 6 | 2 |
| Malaysia | 3 | 1 |
| Mexico | 2 | 0.7 |
| Guatemala | 2 | 0.7 |
| South Korea | 1 | 0.3 |
| Chile | 1 | 0.3 |
| Vietnam | 1 | 0.3 |

State distribution of US isolates

| State | # isolates | % of US isolates¹ |
|---|---|---|
| California | 47 | 37 |
| Florida | 28 | 22 |
| South Carolina | 15 | 12 |
| Alabama | 11 | 8.7 |
| Georgia | 7 | 6 |
| New York | 6 | 5 |
| North Carolina | 3 | 2 |
| Minnesota | 3 | 2 |
| Indiana | 3 | 2 |
| Nebraska | 2 | 2 |
| Ohio | 2 | 2 |

Age distribution

| Age range | # isolates | % of total panel¹ |
|---|---|---|
| 0 to 10 | 10 | 3.3 |
| 11 to 20 | 12 | 4.0 |
| 21 to 30 | 12 | 4.0 |
| 31 to 40 | 25 | 8.3 |
| 41 to 50 | 28 | 9.3 |
| 51 to 60 | 27 | 9.0 |
| 61 to 70 | 53 | 18 |
| 71 to 80 | 62 | 21 |
| 81 to 90 | 42 | 14 |
| 91 to 100 | 11 | 3.7 |
| not specified | 18 | 6.0 |

Gender distribution

| Gender | # isolates | % of total panel¹ |
|---|---|---|
| Female | 186 | 62.0 |
| Male | 114 | 38.0 |

Sampling time distribution

| Collection year | # isolates | % of total panel¹ |
|---|---|---|
| 2017 | 30 | 10. |
| 2018 | 3 | 3 |
| 2019 | 259 | 86.3 |
| 2020 | 2 | 0.7 |

Sampling site distribution

| Body location | # isolates | % of total panel¹ |
|---|---|---|
| Urine | 254 | 84.7 |
| Blood | 26 | 8.7 |
| Bladder | 17 | 5.7 |
| Urethra | 2 | 0.7 |
| Kidneys | 1 | 0.3 |

MDR distribution

| MDR status² | # isolates | % of total panel¹ |
|---|---|---|
| MDR | 115 | 38.3 |
| not MDR | 185 | 61.7 |

¹Due to rounding, not all percentages will sum to 100%.
²MDR = multidrug-resistant. Strains were defined as MDR if they were resistant to three or more classes of antibiotics.

Bacteria plates for both host range assays were created by growing the bacterial strains to log phase for approximately 4 to 6 hours in a 37° C. shaking incubator. Those cultures were then diluted in LB with glycerol to a target final optical density at 600 nm (OD₆₀₀) of 0.02 in 20% glycerol before the panels were frozen. After further testing, the average titer of strains on the panel was determined to be 2.89×10⁶ colony-forming unit (CFU)/mL. The panels were stored in the −80° C. freezer until use for host range testing.

Individual phages from CK618 were tested at maximum available titer. Additionally, each phage in CK618; Table 15) was combined into cocktails with three different phage concentrations.

TABLE 15
Summary of CK618

| | | CK618 | |
|---|---|---|---|
| | | Engineering | Titer |
| Phage | Genus | Status | (PFU/mL) |
| p00exe014 | Tequatrovirus | Full Construct | 5.0 × 10¹⁰ |
| p004Ke009 | Tequatrovirus | Full Construct | 2.1 × 10¹⁰ |
| p00jc | Phapecoctavirus | Wild Type | 2.7 × 10¹¹ |
| p00ke | Unclassified Myovirus | Wild Type | 3.2 × 10¹⁰ |
| p00c0e030 | Mosigvirus | Full Construct | 4.8 × 10¹⁰ |
| p5516 | Vectrevirus | Wild Type | 3.7 × 10¹⁰ |

For the highest titer cocktail, an equal volume of each phage was combined to produce a cocktail with a total cocktail titer of 1.0×10¹¹ PFU/mL. For two lower titer cocktails, the phage titers were normalized before combination to produce cocktails with each phage at a titer of 3.0×10⁹ PFU/mL (total cocktail titer of 1.8×10¹⁰ PFU/mL) or 1.0×10⁷ PFU/mL (total cocktail titer of 6.0×10⁷ PFU/mL). Phages were produced through the process development (PD) process. The PD process uses the same general manufacturing process used for clinical trial material manufacturing except at a smaller scale. Both potency and purity are evaluated for consistency with clinical trial material including endotoxin levels. The cocktails were then subjected to host range testing via a liquid-based assay and a plaquing-based assay on the 300-isolate CP described above with an additional 4 strains of interest.

### Liquid-Based Host Range

Frozen bacterial panels were pulled from the freezer at least 45 minutes before the start of the Liquid Host Range protocol on the Hamilton VANTAGE liquid handling robot. The instrument was prepared with all of the necessary consumables, including LB amended with 10 mM CaCl₂ and 10 mM MgCl₂ (hereafter referred to as LB+salts) as the growth medium, the bacteria panels (up to 4 different panels in 96 well microtiter plates), and phage lysates in Eppendorf tubes. Blank samples with LB+salts only were included in the 96-well microtiter plates and in the Eppendorf tubes to test for potential contamination of the run and as a control. Each well of a 96-well microtiter plate was loaded by the liquid-handling robot with 40 μL of LB+salts, then 5 μL of phage, then 5 μL of bacteria from the bacterial panel. This preparation produced a final phage concentration in the test sample 1/10 the concentration of the input phage. Once all combinations were prepared, the plates were put into the shaking incubator at 37° C. Plates were incubated for 20 hours, and the OD600 of each well in each plate was read every hour.

The aforementioned protocol generates growth curves for each bacterial strain in the presence and absence of the phage cocktail. The area under the curve (AUC) is computed, and the ratio of (AUC in the presence of phage)/(AUC in the absence of phage) is calculated (FIG. 19, left panel). If the AUC ratio is <0.65, the strain is considered to be targeted by the phage cocktail. The number of strains targeted divided by the number of strains tested gives the host range percentage, or % HR. The same analysis was performed on the subset of strains that are classified as multidrug-resistant (MDR). Additionally, some strains that are targeted by the phage cocktail eventually develop resistance and begin to grow, thus increasing the OD₆₀₀ reading in that well. Other strains remain suppressed until the end of the assay time course (20 hours). Strain growth was quantified by examining the amount of time in hours for a given strain+phage sample to reach an OD₆₀₀ of 0.4 (FIG. 19 Error! Reference source not found., right panel). If the sample never reached an OD₆₀₀ of 0.4, that sample was considered suppressed. The number of strains that remain suppressed to below an OD₆₀₀ of 0.4 divided by the number of strains tested gives the suppression metric % OD<0.4.

### Plaquing-Based Host Range

Frozen bacterial panels were removed from the freezer to thaw, and 1 mL of LB was added to each well of the deep well block to incubate overnight in a 37° C. shaking incubator. Once the cultures had grown overnight, they were used to produce the overlays needed for the double agar overlay method. To create these overlays, rectangular LB agar plates were used with an overlay mixture of 100 μL of bacteria and 0.56% low salt top agar (low salt top agar mixed 3:1 with LB) poured over the plate. The overlays were poured onto the agar plates and left to solidify for at least 20 minutes before spotting.

Phage plaquing was performed using the Hamilton VANTAGE liquid handling robots. Each phage (at maximum available titer, Table 15) or phage cocktail (as described in “Materials and Methods” section) was serially diluted 1:10 in LB. After solidification of the double agar overlay, phage were spotted onto the overlay by the robots. Once spotting was completed and the spots had dried, the plates were taken from the liquid handling robot and placed in a 37° C. incubator overnight.

After an overnight incubation, the plates were imaged using an integrated camera, IDS uEye, on the liquid handling robot Hamilton STAR. An in-house algorithm was used to identify sensitive bacterial strains by counting the number of individual plaque forming units. A minimum of three plaque forming units was used to designate a hit. Plates giving outlier results (>2 log difference between three methods in the algorithm) and plates with no plaques detected by the algorithm were counted manually.

### Results

Host range of the phage cocktail was tested using two methods which generate AUC after phage exposure in liquid and plaquing on bacterial overlays. The combination of these two metrics demonstrated that at max feasible titer CK618 targets 92.4% of the strains in the CP. On the MDR strain subset which represents 38.8% of the CP, the host range percentage increased to 94.1% for CK618.

An additional metric used to determine a cocktail's performance is Time to OD. This metric is useful for determining how effective a cocktail is at suppressing growth of the bacterial strains after the initial decrease. The score considers the percentage of strains to not rebound to OD≥0.4 after the initial decrease in OD. Therefore, a higher Time to OD percentage represents those strains that were suppressed by the cocktail. The Time to OD score for CK618 was 67.8% at max feasible titer on the full panel and 62.4% for MDR strains (Table 16).

TABLE 16
Host range of CK507, CK618, and individual phages by different measurement methods

| | | p00exe014 | p004ke009 | p00c0e030 | p00ke | p00jc | p5516 | CK618 | CK618 | CK618 |
|---|---|---|---|---|---|---|---|---|---|---|
| | | 5.0 × | 2.1 × | 4.8 × | 3.2 × | 2.7 × | 4.3 × | 1.0 × | 1.8 × | 6.0 × |
| | | 10⁹ | 10⁹ | 10⁹ | 10¹⁰ | 10¹⁰ | 10⁸ | 10¹⁰ | 10⁹ | 10⁶ |
| Metric | Measurement | PFU/mL | PFU/mL | PFU/mL | PFU/mL | PFU/mL | PFU/mL | PFU/mL | PFU/mL | PFU/mL |
| % HR | Liquid | 67.9 | 39.5 | 4.3 | 37.7 | 38.7 | 24.8 | 82.9 | 78.0 | 71.7 |
| | Plaquing | 77.3 | 51.3 | 71.7 | 47.7 | 51.6 | 38.8 | 91.5 | 91.5 | 91.5 |
| | Liquid OR | 81.6 | 57.2 | 71.7 | 67.1 | 55.3 | 54.4 | 93.6 | 92.8 | 92.2 |
| | plaquing | | | | | | | | | |
| % OD < 0.4 | Liquid | 49.8 | 20.9 | 1.2 | 18.4 | 16.4 | 4.9 | 67.8 | 59.2 | 50.5 |
| MDR % | Liquid | 63.4 | 26.9 | 2.7 | 37.1 | 32.3 | 26.3 | 82.8 | 74.2 | 69.4 |
| HR | Plaquing | 78.5 | 37.6 | 72.0 | 31.2 | 33.3 | 41.9 | 90.3 | 90.3 | 90.3 |
| | Liquid OR | 81.7 | 42.5 | 72.0 | 55.9 | 39.3 | 60.8 | 94.1 | 92.5 | 90.3 |
| | plaquing | | | | | | | | | |
| MDR % | Liquid | 49.5 | 12.4 | 1.1 | 19.4 | 12.9 | 7.5 | 62.4 | 54.8 | 51.1 |
| OD < 0.4 | | | | | | | | | | |

### Example 12. In Vitro Synergy with Antibiotics

The objective of this study was to assess the interaction of CK618 with antibiotics.

### Materials and Methods

Phage cocktail CK618 was tested in combination with and alongside antibiotics against two isolate panels. The first assay used a panel of 88 contemporaneous urinary tract infection (UTI) isolates from North America, Europe, Latin America, and Asia. Twenty-one (24%) of those isolates are classified as multidrug-resistant (MDR), with resistance to 3 or more antibiotic classes. Seven different antibiotics were tested against this panel, representing five standard-of-care (SOC) antibiotics (ceftriaxone, cephalexin, trimethoprim/sulfamethoxazole [TMP/SMX, also called cotrimoxazole], fosfomycin, and nitrofurantoin) and two non-SOC antibiotics (cefdinir and ciprofloxacin) that are commonly used as comparators. The second assay used a panel made up of a 300-strain clinical isolate panel (Table 14) with an additional 4 strains of interest. Ninety-three (31%) of those isolates are classified as MDR. Four of the same SOC antibiotics tested against the 88-isolate panel were tested against this panel. The non-SOC antibiotics were not included due to their limited use in clinical settings, and nitrofurantoin was not included due to the low probability of resistance to this drug.

For both isolate panels, antibiotic concentrations tested were determined by using Clinical and Laboratory Standards Institute (CLSI) or European Committee on Antimicrobial Susceptibility Testing (EUCAST) breakpoints at concentrations that kill susceptible isolates but allow intermediate or resistant isolates to grow in the standard AST microdilution assay (Table 17).

TABLE 17
Antibiotic and Phage Treatment Conditions

| Antibiotic or Phage | | Concentration | | |
|---|---|---|---|---|
| Cocktail | Class | in Assay | | Breakpoint Reference |
| Cefdinir | 3^{rd} gen cephalosporin | 1 | μg/ml | CLSI M100 Table 2A |
| Ceftriaxone | 3^{rd} gen cephalosporin | 1 | μg/ml | CLSI M100 Table 2A |
| Cephalexin | 1^{st} gen cephalosporin | 16 | μg/ml | EUCAST Breakpoint Table v |
| | | | | 11.0 |
| Ciprofloxacin | Fluoroquinolone | 0.25 | μg/ml | CLSI M100 Table 2A |
| Trimethoprim/ | Folate pathway | 2 | μg/ml | CLSI M100 Table 2A |
| Sulfamethoxazole | | | | |
| (TMP/SMX) | | | | |
| Fosfomycin | Fosfomycin | 8 | μg/ml | EUCAST Breakpoint Table v |
| | | | | 11.0 (uncomplicated UTI only) |
| Fosfomycin | Fosfomycin | 64 | μg/ml | CLSI M100 Table 2A |
| Nitrofurantoin | Nitrofurans | 32 | μg/ml | CLSI M100 Table 2A |

| | | | |
|---|---|---|---|
| LBP-EC01 | Bacteriophage Cocktail | 6 × 10⁶ PFU/ml total, | Not applicable |
| | | 1 × 10⁶ PFU/ml per phage | |

CLSI = Clinical and Laboratory Standards Institute; EUCAST = European Committee on Antimicrobial Susceptibility Testing; PFU = plaque-forming unit; SMX = sulfamethoxazole; TMP = trimethoprim

The composition of the proposed Phase 2 CRISPR-enhanced phage (CRISPR-phage, hereafter termed ‘crPhage’) cocktail (CK618) has been updated from the CK570 to include six phages as compared to three phages in CK570. Phage cocktail CK618, was prepared such that all phages had equivalent titers in the final cocktail. The cocktail was then mixed with bacteria to obtain final titers of 1×10⁶ plaque-forming unit (PFU)/mL/phage for the cocktail and 1×10⁵ colony-forming unit (CFU)/mL for the bacteria, resulting in a multiplicity of infection (MOI) of 10 for each phage in a sample. Briefly, 5 μl of each sample (CK618 alone, CK618 and antibiotics, antibiotics alone, or medium alone) was added to 5 μl of optical density (OD)-normalized bacterial isolate and 40 μl LB medium. Reactions were incubated at 37° C. with shaking and OD measurements were taken each hour for 20 hours.

Host range percentage is the percent of isolates that responded to treatment of antibiotics or CK618 In the case of the 88-isolate panel, results were analyzed on the complete panel as well as the subset of classified as multidrug-resistant (MDR) (n=21) and beta-lactam-resistant (n=31). In the case of the 304-isolate panel, results were analyzed on the complete panel as well as the subset of isolates classified as MDR (n=93) and beta-lactam-resistant (n=91).

### Results

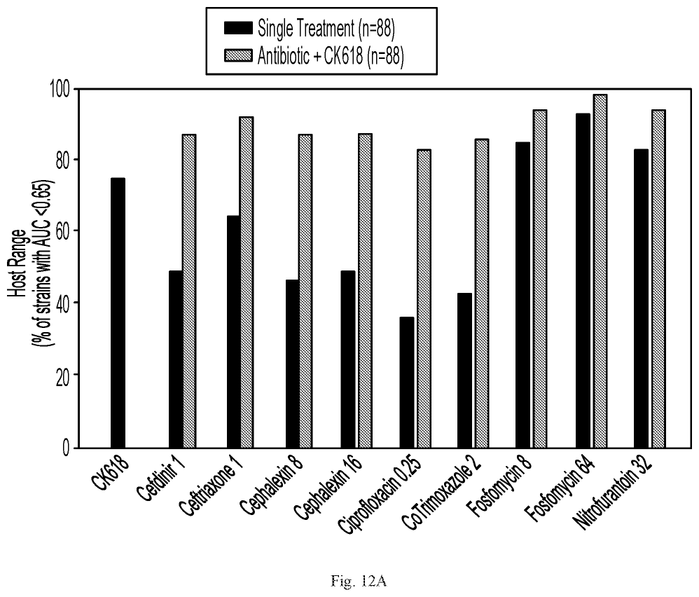

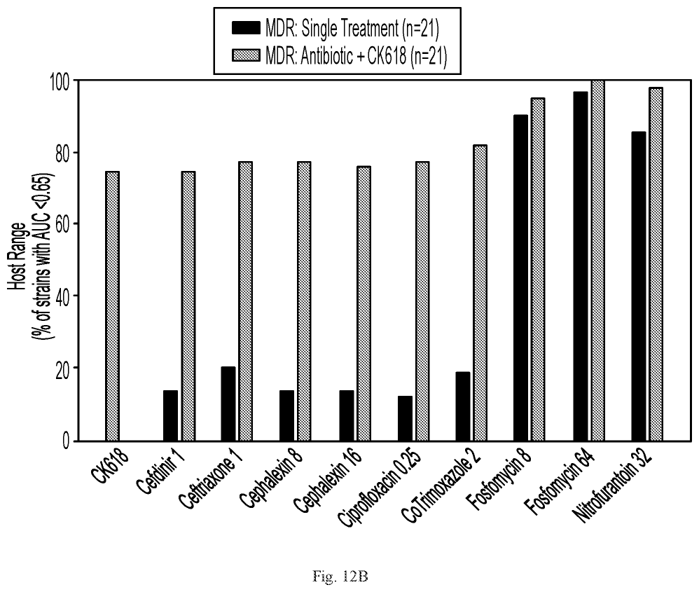

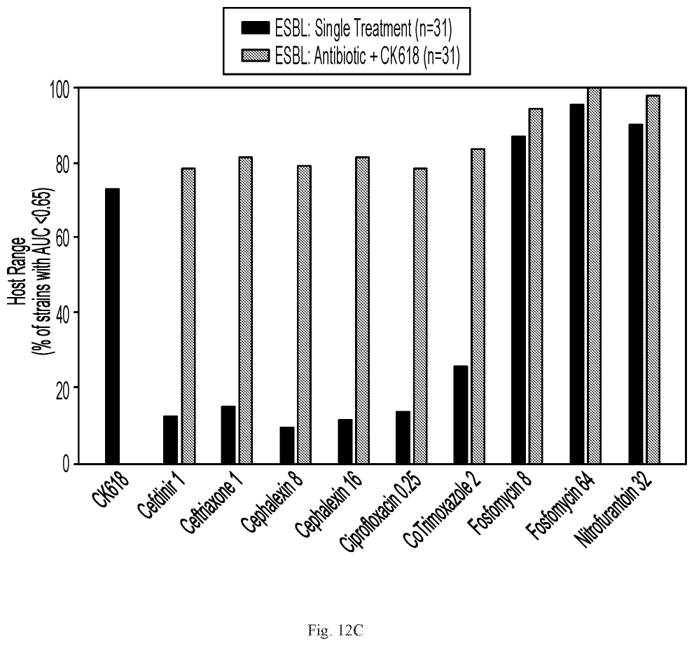

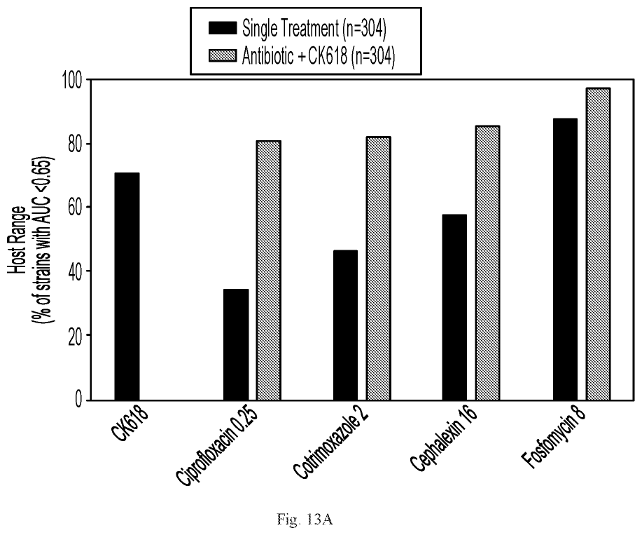

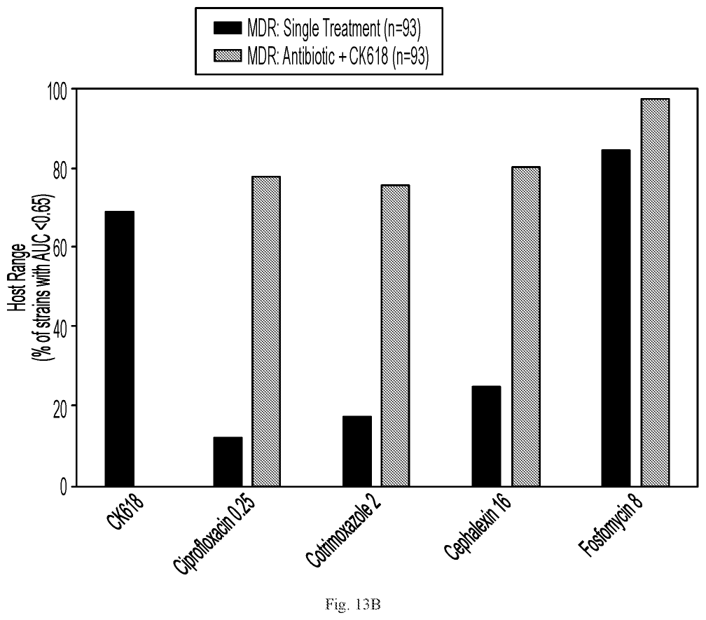

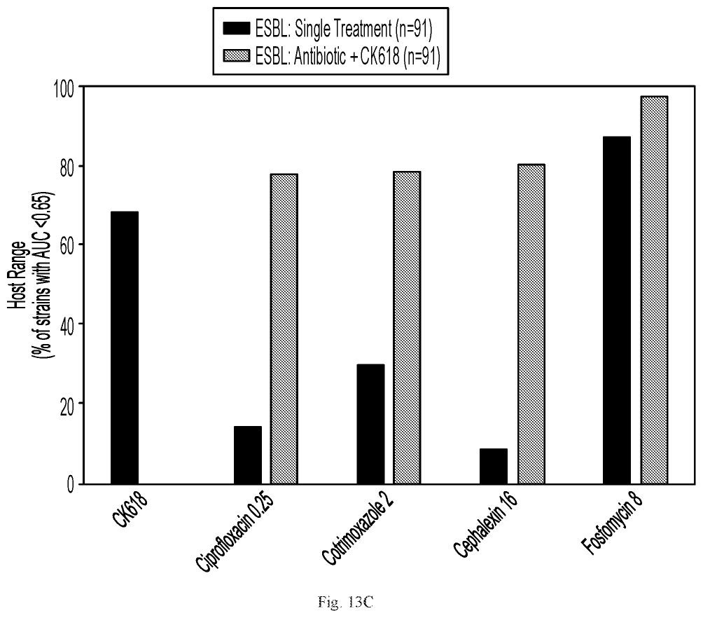

CK618 outperformed most SOC antibiotics, including first and third generation cephalosporins, ciprofloxacin, and TMP/SMX on all strains (FIGS. 12A-12C, FIGS. 13A-13C, Tables 18, 19). Combination therapy was more effective in all cases compared with antibiotic treatment alone. In the MDR and beta-lactam-resistant subsets of strains, combination therapy with the cephalosporins, ciprofloxacin, or TMP/SMX performs similarly to, or slightly better than, the phage cocktail alone. Fosfomycin and nitrofurantoin had strong activity across all strains and were minorly improved by the addition of LBP-EC01. For MDR and beta-lactam-resistant strains, the overall host range of cephalosporins, ciprofloxacin, and TMP/SMX was reduced while LBP-EC01, Nitrofurantoin, and fosfomycin performed similarly regardless of antibiotic resistance status across the isolates tested (FIGS. 12A-12C, FIGS. 13A-13C, Tables 18, 19).

TABLE 18
Host Range of CK618and Antibiotics Alone and in Combination on 88-Isolate Panel

| | Host Range % on all | | Host Range % on MDR | | Host Range % on Beta-lactam- | |
|---|---|---|---|---|---|---|
| | Strains (n = 88) | | Subset (n = 21) | | Resistant Subset (n = 31) | |
| | Single | Combination: | Single | Combination: | Single | Combination: |
| | agent | Antibiotic + | agent | Antibiotic + | agent | Antibiotic + |
| Treatment | treatment | CK618 v.2 | treatment | CK618 v.2 | treatment | CK618 v.2 |
| CK618 | 74.6 | n/a | 74.6 | n/a | 73.1 | n/a |
| Cefdinir | 48.9 | 87.5 | 14.3 | 74.6 | 12.9 | 78.5 |
| Ceftriaxone | 64.4 | 92.4 | 20.6 | 77.8 | 15.1 | 81.7 |
| Cephalexin | 49.2 | 87.9 | 14.3 | 76.2 | 11.8 | 81.7 |
| Ciprofloxacin | 36.4 | 83.0 | 12.7 | 77.8 | 14.0 | 78.5 |
| TMP/SXT | 42.8 | 85.6 | 19.0 | 82.5 | 25.8 | 83.9 |
| Fosfomycin 8 | 85.2 | 93.9 | 90.5 | 95.2 | 87.1 | 94.6 |
| Fosfomycin 64 | 93.2 | 98.5 | 96.8 | 100.0 | 95.7 | 100.0 |
| Nitrofurantoin | 83.0 | 93.9 | 85.7 | 98.4 | 90.3 | 97.8 |

MDR = multidrug-resistant

TABLE 19
Host Range of CK618 and Antibiotics Alone and in Combination on 304-Isolate Panel

| | | | | | Host range % on beta- | |
|---|---|---|---|---|---|---|
| | Host range % on all | | Host range % on MDR | | lactam-resistant subset | |
| | strains (n = 304) | | subset (n = 93) | | (n = 91) | |
| | Single | Combination: | Single | Combination: | Single | Combination: |
| | agent | Antibiotic + | agent | Antibiotic + | agent | Antibiotic + |
| Treatment | treatment | LBP-CK618 | treatment | LBP-CK618 | treatment | LBP-CK618 |
| CK618 | 70.7 | n/a | 68.8 | n/a | 68.1 | n/a |
| Ciprofloxacin 0.25 | 34.2 | 80.9 | 11.8 | 77.4 | 14.3 | 75.8 |
| Cotrimoxazole 2 | 46.4 | 81.9 | 17.2 | 75.3 | 29.7 | 78.0 |
| Cephalexin 16 | 57.6 | 85.5 | 24.7 | 79.6 | 8.8 | 79.1 |
| Fosfomycin 8 | 87.5 | 97.0 | 83.9 | 96.8 | 86.8 | 96.7 |

These data demonstrate that CK618 outperformed all of the antibiotics tested with the exception of the revived drug Fosfomycin, which already has a few demonstrated cases of antibiotic resistance mechanisms and is efficacious currently only because it has not been widely used until the emergence of MDR/XDR isolates. Additionally, CK618 acts in a complementary fashion with antibiotics. Regardless of isolate MDR status or antibiotic identity, in all cases concurrent treatment with CK618 plus antibiotic demonstrates a higher host range than treatment with either CK618 or antibiotic alone. In the case of Bactrim (TMP/SMX or cotrimoxazole), the percent of MDR strains from the 304-isolate panel targeted increases from 17.2% when treated with Bactrim alone to 75.3% when treated with Bactrim+CK618 (Table 19), providing a strong argument for the benefit of concurrent treatment.

### Example 13. Urinary Tract Infection Model to Test Cocktail Efficacy

A urinary tract infection (UTI) model was developed in C3H/OuJ mice infected with Escherichia coli (E. coli) strain ATCC 700928 that resulted in stable infection of the bladder and kidneys with E. coli. CK618 surrogate cocktail CK570 was tested in this study to determine the extent to which the cocktail can reduce E. coli burden in bladder, kidneys, and urine.

### Materials

The CRISPR phage (‘crPhage’) cocktail included 6 phages (p004k, p00jc, p00c0, p00ke, p5516 and p00ex). The full construct version of p00jc used in CK570 uses Cpf1/Cas12a as the CRISPR system rather the PAIC Cas operon used in other full construct phages, and was produced through the process development (PD) process. The PD process uses the same general manufacturing process used for clinical trial material manufacturing except at a smaller scale. Both potency and purity are evaluated for consistency with clinical trial material including endotoxin levels.

TABLE 22
Components of Surrogate Cocktail CD570

| | | Engineering |
|---|---|---|
| Phage | Genus | Status |
| p00ex | Tequatrovirus | Full Construct |
| p004k | Tequatrovirus | Full Construct |
| p00jc | Phapecoctavirus | Full Construct¹ |
| p00ke | Unclassified Myovirus | Wild Type |
| p00c0 | Mosigvirus | Full Construct |
| p5516 | Vectrevirus | Wild Type |

1 The full construct version of p00jc used in CK570 uses Cpf1/Cas12a as the CRISPR system rather than PAIC Cas operon used in the other full construct phages.

CK570 was formulated in 1× tris buffered saline (TBS), with a composite potency of 7.6×10¹⁰ plaque-forming units (PFU)/mL and an estimated endotoxin content of 16.6 EU/mL. Mice that were treated with CK570 received 3.8×10⁹ PFU/dose via intraurethral (IU) administration or 7.6×10⁹ PFU/dose via IV administration. The cocktail potency relative to the infection strain was 2.0×10¹⁰ PFU/mL (as determined by serial dilution and plating on ATCC 700928 bacterial overlays). The vehicle used in this study was 1×TBS. Ciprofloxacin was a positive control and was prepared as an injectable by a compounding pharmacy as a 2.5 mg/mL stock in water.

C3H/OuJ female mice, aged approximately 8 weeks and weighing 21 to 24 grams, were used in this study. Mice were housed with 1 to 5 animals per cage and were provided food (5P76 Prolab isopro irradiated lab diet) and water ad libitum.

### Experimental Design

On Day 0, animals in Groups 1 through 5 received a single IU dose of 50 μL of E. coli (as described below). Forty-eight (48), 60, 72, 84, and 96 hours post-infection (p.i.), all animals received vehicle (1×TBS), CK570, or ciprofloxacin administered IU (Groups 1 and 2) or IV (Groups 3 through 5) (Table 20)

TABLE 20
Experimental design

| | | Day 0 Infection | 48 hours Post-infection Treatment | | | |
|---|---|---|---|---|---|---|
| | | Bacteria, Dose, | Test Article | ROA and | Dosing | Experimental |
| Group | # | ROA, & Volume | and Dose | Volume | Schedule | Endpoints |
| 1 | 15 | E. coli | TBS | IU 50 μL | 48, 60, 72, | Urine, bladder, |
| | | (b3675) | | | 84 and | blood, kidneys, |
| 2 | 15 | 1 × 10⁹ | CKS70 7.6 × 10¹⁰ | | 96 hours | and spleen |
| | | CFU IU | PFW/mL | | post- | were collected |
| 3 | 15 | 50 μL | TBS | TV 100 μL | infection | from n = 5 |
| 4 | 15 | | CKS70 7.6 × 10¹⁰ | | | mice/ |
| | | | PFU/mL | | | timepoint |
| 5 | 15 | | Ciprofloxacin 5 mg/mL | | | |

CFU = colony forming unit; IUs = intraurethral, IV = intravenous; PPU = plaque forming unit, ROA = route of administration; TBS = tris buffered saline. Cocktaildose & Ested as composite potency.

Briefly, on Day 0, Time 0, mice were infected with E. coli (ATCC 700928) at 109 colony-forming units (CFU) by IU administration (FIG. 14A). Mice were anesthetized with 1-4% isoflurane in oxygen for 2-3 minutes until a deep plane of anesthesia was achieved, confirmed by toe pinch reflex. Each mouse was then placed on a board, ventral side up, with the nose inserted into a nose cone supplying isoflurane. The lower abdomen was gently massaged to expel any urine in the bladder. Using a 24 G neonatal catheter (BD Insyte-N Autoguard BC 24 GA 0.56IN [0.7×14 mm] cat #381411) connected to a 1 mL syringe, 50 μL of inoculum was slowly instilled into the bladder. For IV administration, mice were manually restrained in a restrainer device, and study treatment was administered via the lateral tail vein. A dose volume of 100 μL was used for IV injections. For IU administration, the same method was used as for the original administration of E. coli. A dose volume of 50 μL was used for IU administration.

Two cohorts of mice were then euthanized, and E. coli burden was assessed by serial dilution and culture from homogenized tissues at 49 hours and 54 hours p.i. (1 hour and 6 hour post-treatment, respectively). A third cohort of mice received 4 additional doses of either vehicle, ciprofloxacin, or CK570 every 12 hours until 96 hours p.i. At 102 hours p.i. (6 hours post-final treatment), the remaining mice were euthanized, and E. coli burden enumerated. Following anesthesia with inhaled isoflurane (1-5%), the abdomen was opened to expose the bladder and urine was aseptically collected using an insulin syringe and placed into a sterile, DNAse/RNAse-free tube.

Following urine collection, the bladder was removed and placed into a pre-weighed homogenization tube containing 1.4 mm ceramic beads (Fisher Scientific cat #15-340-153) and 200 μL of sterile phosphate buffered saline (PBS; Teknova cat #P0200) and re-weighed to the thousandths place. Kidneys and spleen were collected in a manner similar to bladder into a volume of 1 mL of sterile PBS. Blood was collected by inserting a needle into the heart and collecting into a 4 mL sodium heparin blood collection tube (Fisher Scientific cat #02-689-5) and gently rotating to ensure adequate mixing with anti-coagulant. Tissues were homogenized, and all samples immediately serially diluted and plated for CFU enumeration. CFU/mL calculations were normalized to gram tissue for bladder and kidneys. All CFU data were log-transformed.

### Results

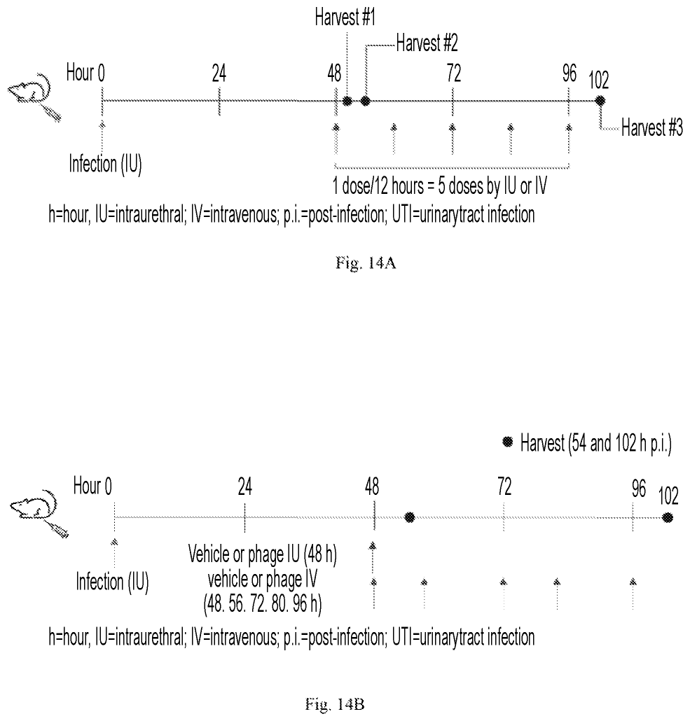

IU administration of CK570, which directly delivered the cocktail to the site of infection, resulted in significant reduction of bacterial burden in kidneys at all timepoints, in bladder at 54 hours p.i. and 102 hours p.i., and in urine at 49 hours p.i. and 102 hours p.i. (FIGS. 15A-15C). Reduction in bacterial burden ranged from −2 log to −4 log, and in some cases reached the limit of detection (LOD). These data also demonstrate that significant efficacy is achievable with either a single dose or with 5 doses given twice daily (BID).

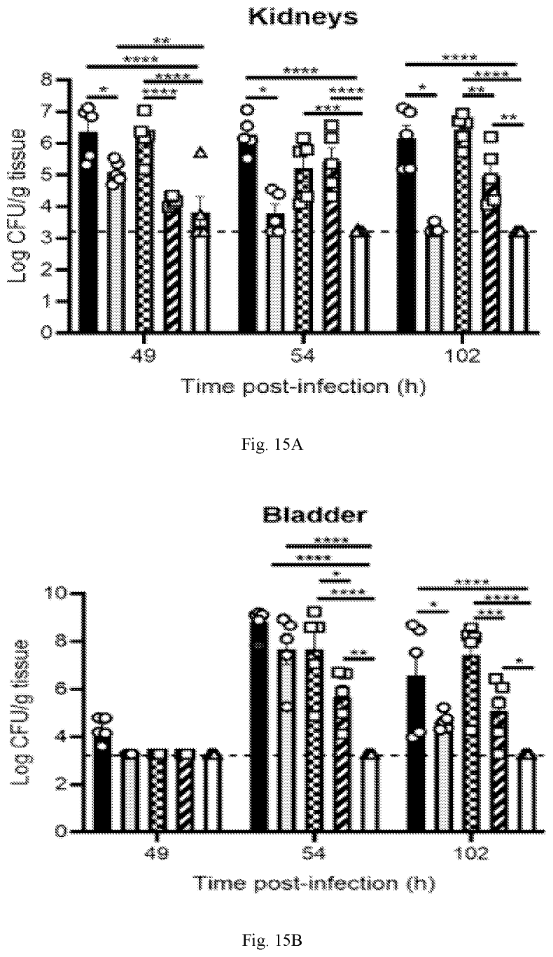

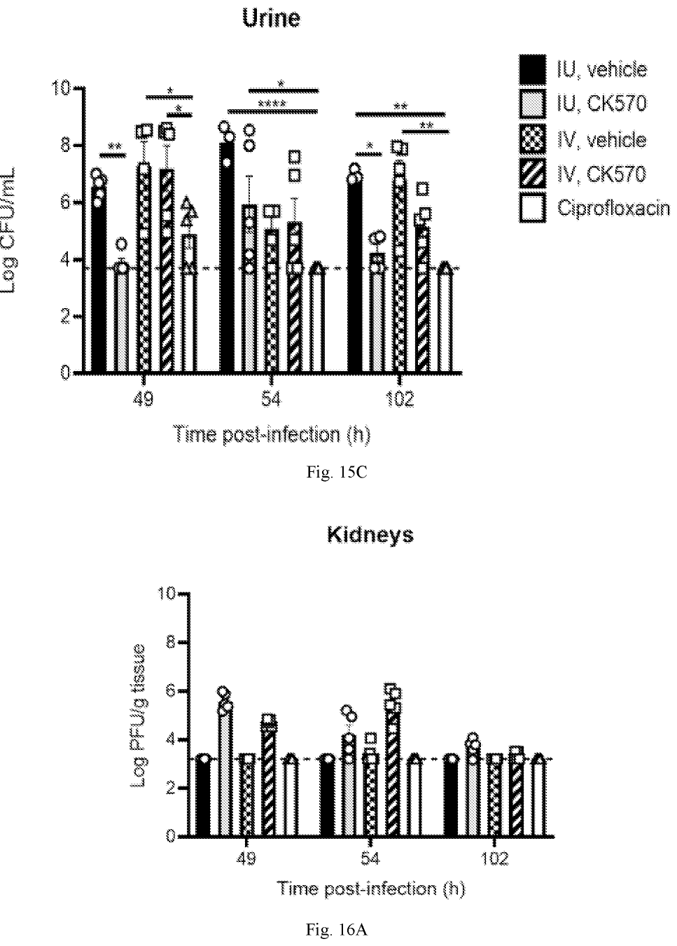

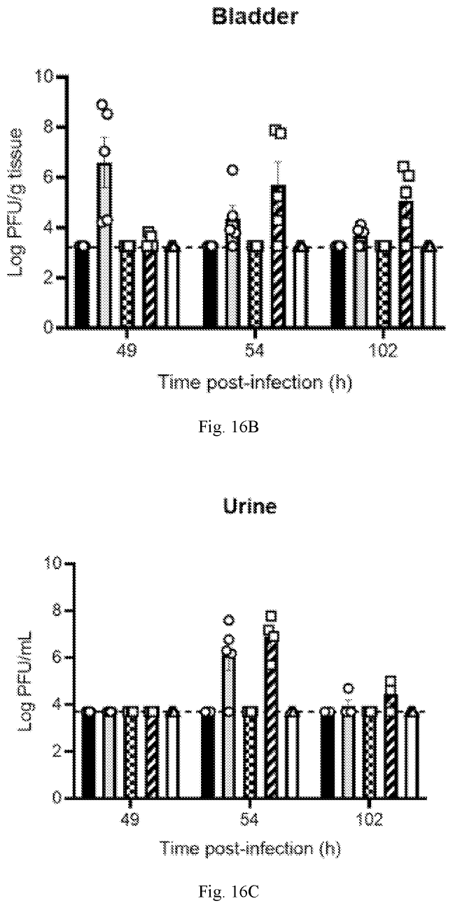

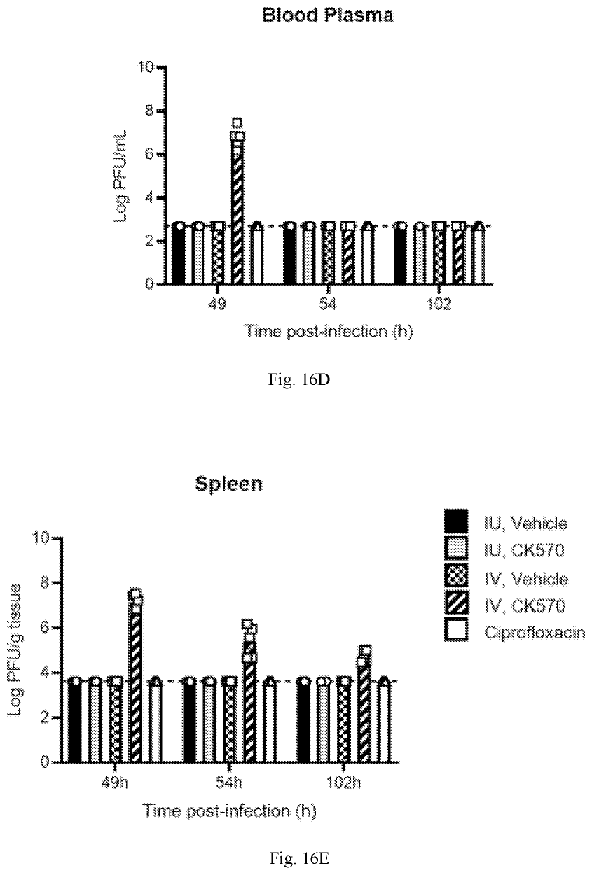

Importantly, intravenous (IV) administration of CK570, a systemic route of administration, also achieved significant reduction of bacterial burden. Significant decreases in enumerated CFU levels were observed in kidneys at all timepoints, in bladder at 54 hours p.i. and 102 hours p.i., and in urine at 102 hours p.i (FIGS. 16A-16C). Reduction in bacterial burden ranged from ˜2 log to ˜3 log. Ciprofloxacin treatment significantly reduced bacterial burden to the LOD in each tissue, across all timepoints assessed. Of note, ciprofloxacin is not a standard-of-care antibiotic for human uncomplicated UTI, and the use of ciprofloxacin in this experiment was intended solely as a positive control to confirm that that the model was executed as expected. In addition to enumeration of CFU, CK570 phages were also enumerated by serial dilution and plaquing of filtered tissue homogenates, filtered plasma, and urine on overlays of ATCC 700928. IU administration of phage resulted in detectable levels of CK570 in kidneys, bladder, and urine, especially at early timepoints. IV administration also resulted in detectable levels of LBPEC01 in blood and spleen at early timepoints, but signal dropped to at or near the limit of detection by 102 hours p.i. Despite the lowest levels of detectable phages at 102 hours p.i., there was still a significant cocktail efficacy, as evidenced by significant reduction in bacterial burden by both routes of administration. Taken together, IU or IV administration of CK570 results in sufficient delivery of the cocktail to the key organs to achieve efficacy.

Therefore, Both IU and IV administration were effective routes of administration to achieve reduction in bacterial burden, demonstrating that systemic delivery of CK570 is a viable clinical route of administration. Moreover, efficacy was achieved after a single dose of CK570 and maintained following four additional doses. Taken together, these data indicate that CK570 is highly efficacious and is capable of significantly reducing E. coli bacterial burden in a UTI model.

In another study, schematic of the design shown in FIG. 14B, similar results were obtained using CK618. The experimental layout is presented in Table. 21.

TABLE 21
Experimental design

| | | Day 0 Infection | 48 hours Post-infection Treatment | | | |
|---|---|---|---|---|---|---|
| | | Bacteria, Dose, | Test Article | ROA and | Dosing | Experimental |
| Group | # | ROA, & Volume | and Dose | Volume | Schedule | Endpoints |
| 1 | 20 | E. coli | TBS | IU 50 μL | 48 hours post- | 54 and 102 |
| | | (b3675) | | | infection | hours |
| | | 1 × 10⁹ | TBS | IV 100 μL | 56, 72, 80, and 96 | post-infection: |
| | | CFU IU | | | hours post-infection | Bladder, blood, |
| 2 | 20 | 50 μL | TBS | IU 50 μL | 48 hours post- | and kidneys, |
| | | | | | infection | were collected |
| | | | CK618 3.0 × | IV 100 μL | 56, 72, 80, and 96 | from n = 5 |
| | | | 10¹¹ PFU/mL | | hours post-infection | mice/ |
| 3 | 20 | | CK618 3.0 × | IU 50 μL | 48 hours post- | timepoint |
| | | | 10¹¹ PFU/mL | | infection | |
| | | | CK618 3.0 × | IV 100 μL | 56, 72, 80, and 96 | |
| | | | 10¹¹ PFU/mL | | hours post-infection | |

CFU = colony forning unit; IU = intraurethral; IV = intravenous; PFU = plaque forming unit; ROA = route of administration; TBS = tris buffered saline. Cocktaildose is listed ascomposite potency.

### Results

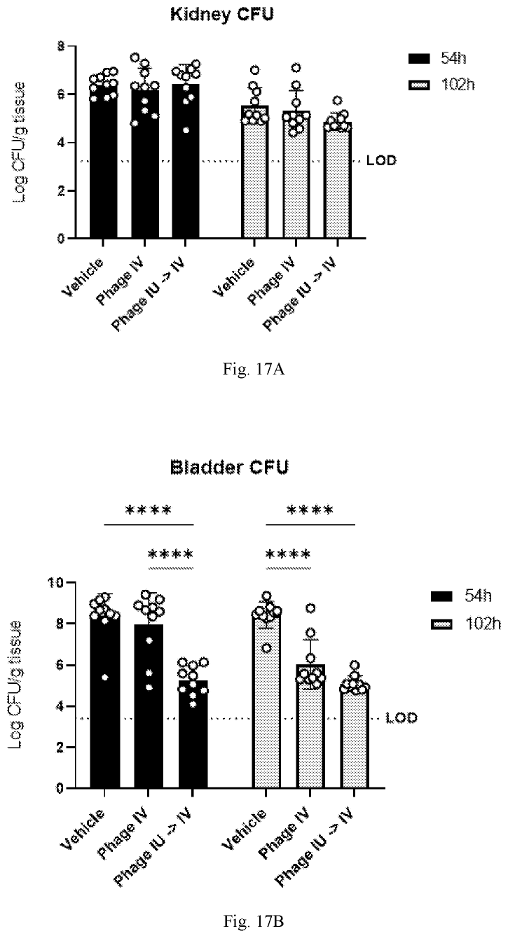

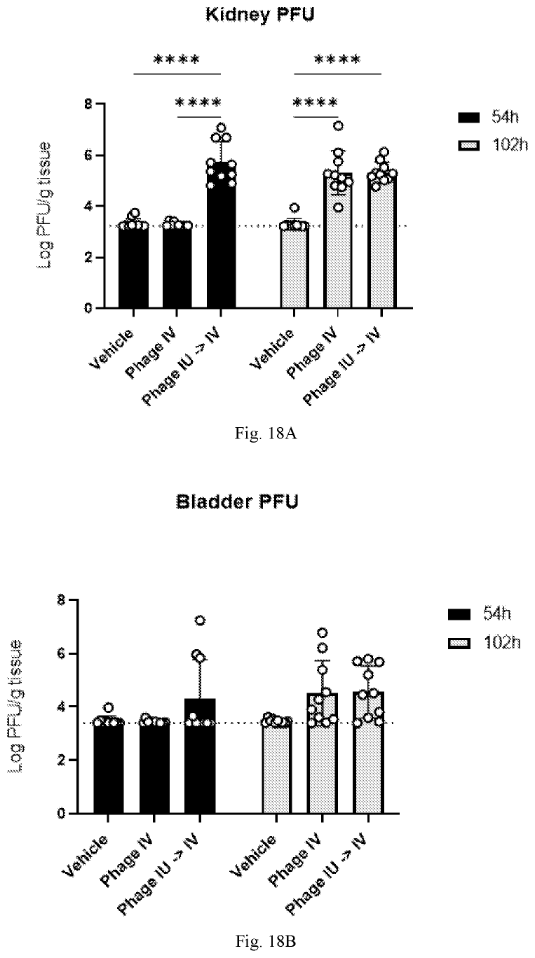

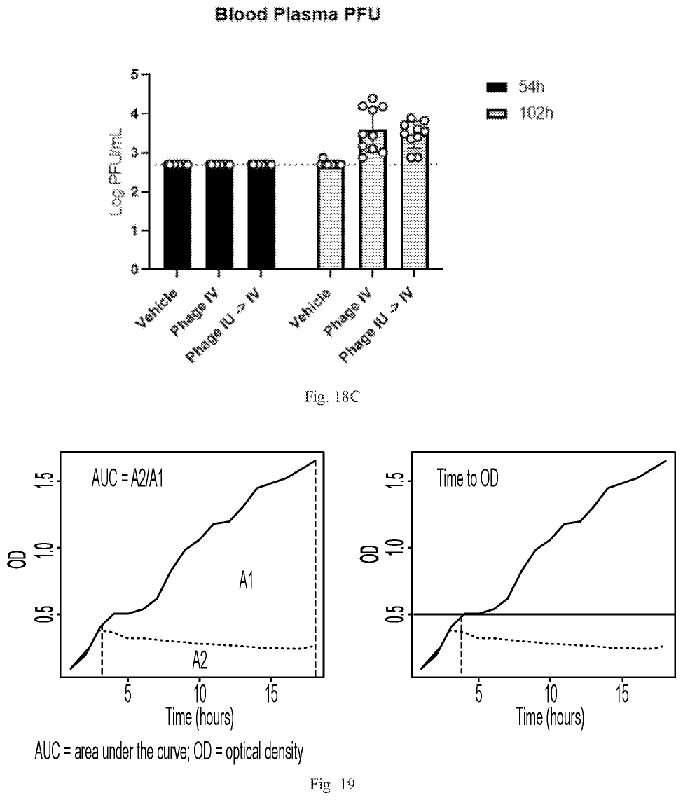

It was observed that in bladder, administration of both an IU dose and an IV dose of CK618 at 48 hours p.i. led to a significant, ˜3 log decrease in CFU at 54 hours relative to either the vehicle treatment or the IV dose alone (FIG. 17B), By contrast, at the 102 hour timepoint, both treatment groups that received 5 IV doses of CK618 showed a statistically significant, 2 to 3 log decrease in CFU relative to the vehicle-treated group, with no significant difference between the groups that did versus did not receive an initial IU dose of CK618. Therefore, an initial IU dose improved the efficacy of a single IV dose of CK618 but did not provide a boost in efficacy relative to five IV doses administered BID. Enumeration of the cocktail phage showed very few detectable phage at 54 hours in the kidneys of animals treated only with a single IV dose of CK618 at 48 hours p.i., whereas phage were clearly detectable in kidneys at the 102 hour timepoint, after 5 IV doses (FIG. 18A). By contrast, animals that received an IU dose at 48 hours p.i. prior to IV dosing had clearly detectable phage in kidneys at both timepoints. In bladder and blood plasma (FIGS. 18B-18C), the IU loading dose did not cause a significant change in biodistribution at either timepoint relative to IV dosing alone. Consistent with the efficacy data, these results suggest that an IU loading dose may improve the biodistribution of phage to the urinary tract relative to a single IV dose, but not relative to 5 BID IV doses.

TABLE 23
Cpf1 sequences

| | | EC STRAIN | |
|---|---|---|---|
| Name | SEQUENCE | COVERAGE % | TARGET |
| Ec_SC_Cpf1_4 | CATTGGTAGCCATGTTTCTTTCCTG | 99.82% | UMP kinase |
| | (SEQ ID NO: 35) | | |
| Ec_SC_Cpf1_5 | ATACTGGAGGAGTCATAAGAATTCG | 99.82% | DNA gyrase |
| | (SEQ ID NO: 36) | | subunit B |
| Ec_SC_Cpf1_7 | CGGCGACCAGTGCCGTAGTATTGAT | 99.81% | 30s ribosomal |
| | (SEQ ID NO: 37) | | protein |
| COMBINED | | 99.93% | |

TABLE 24
Example CRISPR system components

| | SEQ | |
|---|---|---|
| | ID | |
| | NO | SEQUENCE |
| Cpf1 | 46 | AGTATCTATCAGGAATTCGTAAACAAATATTCCCTGTCGAAAACGCTGCGCTTT |
| | | GAATTAATTCCACAAGGCAAAACGCTGGAAAACATCAAAGCGCGTGGCCTTAT |
| | | CCTGGATGACGAAAAGCGTGCAAAAGACTACAAGAAAGCTAAACAAATCATCG |
| | | ACAAATATCATCAGTTTTTTATTGAAGAAATTCTTAGCTCCGTGTGTATCAGTGA |
| | | AGACCTTCTTCAGAACTATAGCGATGTTTACTTCAAGCTGAAGAAATCGGACGA |
| | | TGATAATCTGCAGAAAGATTTTAAGAGTGCCAAGGATACTATCAAGAAACAGA |
| | | TTAGTGAGTACATTAAGGATTCCGAGAAGTTCAAAAACCTGTTCAACCAGAACC |
| | | TGATCGATGCCAAGAAAGGGCAGGAATCCGATCTGATCCTGTGGCTTAAACAA |
| | | TCCAAAGATAACGGCATCGAACTGTTTAAGGCAAATAGCGACATCACAGATAT |
| | | CGATGAAGCACTGGAAATTATCAAATCGTTCAAAGGGTGGACAACCTACTTCA |
| | | AAGGATTTCATGAAAATCGTAAAAACGTGTACAGCTCAAACGACATCCCAACC |
| | | TCTATCATTTACCGCATCGTTGACGATAATTTACCCAAATTTTTGGAGAACAAA |
| | | GCGAAATATGAATCTCTGAAGGATAAGGCGCCGGAAGCGATCAATTATGAACA |
| | | GATCAAAAAGGACTTGGCGGAGGAGTTGACTTTTGATATTGACTATAAAACGTC |
| | | GGAAGTGAACCAGCGCGTCTTTAGCCTGGACGAAGTTTTCGAGATTGCAAACTT |
| | | TAATAACTACTTAAATCAATCCGGGATTACTAAGTTTAATACGATTATTGGCGG |
| | | GAAATTCGTTAACGGTGAAAACACCAAACGTAAAGGTATCAACGAATACATTA |
| | | ATCTGTATAGCCAGCAAATCAACGATAAAACACTCAAAAAATATAAGATGTCC |
| | | GTGCTGTTCAAACAGATTTTAAGTGATACGGAAAGTAAGTCATTTGTGATTGAC |
| | | AAACTGGAAGATGACTCCGATGTAGTGACGACTATGCAGTCATTTTATGAACAG |
| | | ATTGCGGCTTTCAAAACCGTCGAAGAGAAGTCGATCAAAGAGACGCTCAGCCT |
| | | CTTATTTGATGATCTCAAAGCCCAGAAGCTGGATCTTTCGAAAATCTACTTTAA |
| | | AAACGATAAGTCTTTAACCGACCTGTCCCAACAGGTGTTTGATGACTACTCCGT |
| | | GATTGGTACTGCCGTGCTGGAATATATTACCCAGCAAATTGCTCCGAAAAACTT |
| | | AGATAACCCAAGTAAGAAAGAACAGGAACTGATTGCGAAAAAAACGGAAAAA |
| | | GCGAAATATCTGAGCCTGGAAACCATTAAACTGGCCCTGGAAGAATTTAACAA |
| | | ACATCGTGATATCGACAAACAATGCCGTTTCGAAGAAATCCTGGCCAATTTTGC |
| | | CGCCATTCCGATGATTTTCGATGAAATCGCACAGAATAAGGACAATCTGGCGCA |
| | | GATCTCAATTAAATACCAGAATCAGGGTAAGAAAGATCTCCTGCAGGCTAGCG |
| | | CCGAAGATGATGTGAAAGCGATCAAAGACCTGCTGGACCAAACTAATAATCTG |
| | | CTGCATAAACTGAAGATCTTTCACATTAGCCAGTCAGAAGACAAGGCGAACAT |
| | | TCTCGATAAAGATGAACACTTCTACCTGGTTTTCGAAGAATGCTACTTCGAATT |
| | | AGCAAACATTGTCCCCCTCTATAATAAAATTCGCAATTATATTACTCAGAAACC |
| | | CTATAGCGATGAAAAATTCAAACTGAATTTCGAAAACTCCACCCTGGCGAACG |
| | | GTTGGGATAAGAATAAAGAGCCAGATAATACCGCCATTCTGTTTATCAAAGAC |
| | | GATAAATATTATTTAGGCGTCATGAACAAGAAAAACAACAAAATCTTTGACGA |
| | | CAAAGCCATCAAGGAAAATAAGGGTGAAGGTTACAAGAAAATCGTTTATAAAC |
| | | TGCTGCCTGGGGCGAACAAAATGCTGCCGAAAGTGTTTTTTAGCGCCAAGAGC |
| | | ATTAAATTTTATAATCCGAGTGAAGACATCCTGCGCATCCGTAATCATTCGACG |
| | | CATACCAAAAATGGCAGCCCGCAAAAAGGCTATGAAAAGTTTGAGTTCAATAT |
| | | TGAAGATTGTCGTAAGTTTATCGACTTTTACAAACAATCGATCAGCAAACATCC |
| | | CGAATGGAAAGACTTTGGCTTCCGTTTCTCCGACACCCAACGTTATAACTCGAT |
| | | CGATGAGTTCTATCGCGAGGTCGAAAATCAGGGTTATAAACTGACATTCGAAA |
| | | ACATCAGCGAGTCATACATTGACTCCGTTGTAAATCAGGGCAAACTGTACCTCT |
| | | TCCAGATTTATAATAAAGATTTCAGCGCGTACTCAAAGGGCCGCCCCAATCTCC |
| | | ATACCTTATATTGGAAGGCATTGTTTGATGAGCGTAATCTCCAGGATGTAGTGT |
| | | ACAAACTCAATGGCGAGGCGGAACTGTTTTATCGTAAACAGAGCATTCCAAAG |
| | | AAAATTACCCACCCGGCGAAGGAAGCGATTGCCAACAAAAACAAGGACAATCC |
| | | GAAAAAGGAAAGCGTGTTCGAGTACGACCTGATTAAAGACAAACGCTTTACGG |
| | | AAGATAAGTTTTTCTTTCACTGCCCGATCACGATTAACTTTAAATCGTCCGGCGC |
| | | AAACAAATTTAACGACGAAATTAACTTGCTGCTGAAAGAGAAAGCGAACGATG |
| | | TGCACATTCTGTCGATCGATCGTGGTGAACGCCATCTGGCGTATTACACGCTGG |
| | | TGGATGGCAAGGGTAACATTATCAAGCAGGATACCTTCAATATCATCGGTAATG |
| | | ATCGCATGAAAACGAATTATCACGACAAACTGGCTGCTATCGAGAAAGATCGT |
| | | GATAGCGCTCGCAAAGACTGGAAAAAAATCAACAACATCAAGGAAATGAAAG |
| | | AAGGTTATCTTTCTCAGGTAGTACACGAAATCGCCAAATTAGTCATTGAATATA |
| | | ATGCTATTGTAGTGTTTGAAGATCTGAACTTTGGTTTCAAACGCGGTCGCTTCA |
| | | AGGTCGAAAAACAGGTTTACCAGAAATTAGAGAAAATGTTGATTGAGAAACTG |
| | | AACTATCTGGTCTTCAAGGACAATGAGTTTGATAAGACAGGTGGCGTTCTGCGT |
| | | GCGTATCAGCTGACCGCCCCGTTCGAAACCTTCAAGAAAATGGGTAAACAGAC |
| | | GGGTATTATCTATTACGTGCCGGCAGGCTTCACCAGCAAGATTTGCCCGGTCAC |
| | | GGGCTTCGTCAACCAACTGTACCCAAAGTATGAAAGTGTATCCAAGAGCCAGG |
| | | AGTTCTTTAGCAAGTTTGATAAAATCTGCTACAACCTGGATAAGGGCTATTTCG |
| | | AGTTCAGCTTCGACTACAAAAACTTCGGCGACAAAGCCGCGAAAGGCAAGTGG |
| | | ACGATTGCCTCCTTCGGCAGCCGCCTGATTAACTTTCGCAACTCGGACAAAAAT |
| | | CATAATTGGGATACCCGCGAAGTGTATCCTACGAAAGAGCTGGAGAAACTGCT |
| | | CAAAGACTACTCGATCGAATATGGTCACGGTGAGTGTATTAAAGCCGCAATTTG |
| | | CGGTGAAAGCGATAAAAAGTTTTTCGCCAAACTCACCAGCGTATTAAATACTAT |
| | | CCTTCAGATGCGCAATTCCAAGACAGGTACCGAACTCGATTATCTGATCTCGCC |
| | | GGTGGCGGATGTGAACGGGAACTTCTTTGACAGCCGCCAGGCGCCGAAAAATA |
| | | TGCCACAAGACGCAGATGCAAATGGAGCTTATCACATTGGTCTGAAAGGACTT |
| | | ATGCTCCTGGGCCGTATTAAAAATAACCAGGAGGGTAAGAAACTTAATCTGGTT |
| | | ATTAAAAACGAAGAATATTTTGAGTTCGTTCAGAACCGTAACAACTAA |
| Cas3 | 47 | ATGGACGCGGAGGCTAGCGATACTCACTTTTTTGCTCACTCCACCTTAAAGGCA |
| | | GATCGCAGCGATTGGCAGCCTCTGGTCGAGCATCTACAGGCTGTTGCCCGTTTG |
| | | GCAGGAGAGAAGGCTGCCTTCTTCGGCGGCGGTGAATTAGCTGCTCTTGCTGGT |
| | | CTGTTGCATGACTTGGGTAAATACACTGACGAGTTTCAGCGGCGTATTGCGGGT |
| | | GATGCCATCCGTGTCGATCACTCTACTCGCGGGGCCATACTGGCGGTAGAACGC |
| | | TATGGCGCGCTAGGTCAATTGCTAGCCTACGGCATCGCTGGCCACCATGCCGGG |
| | | TTGGCCAATGGCCGCGAGGCTGGTGAGCGAACTGCCTTGGTCGACCGCCTGAA |
| | | AGGGGTTGGGCTGCCACGGTTATTGGAGGGGTGGTGCGTGGAAATCGTGCTAC |
| | | CCGAGCGCCTTCAACCACCGCCACTAAAAGCGCGCCTGGAAAGAGGTTTCTTTC |
| | | AGTTGGCCTTTCTTGGCCGGATGCTCTTTTCCTGCTTGGTTGATGCGGATTATCT |
| | | AGATACCGAAGCCTTCTACCACCGCGTCGAAGGACGGCGCTCCCTTCGCGAGC |
| | | AAGCGCGGCCGACCTTGGCCGAGTTACGCGCAGCCCTTGATCGGCATCTGACTG |
| | | AGTTCAAGGGAGATACGCCGGTCAACCGCGTTCGCGGGGAGATATTGGCCGGC |
| | | GTGCGCGGCAAGGCGAGCGAACTTCCCGGGCTGTTTTCTCTCACAGTGCCCACA |
| | | GGAGGCGGCAAGACCCTGGCCTCTCTGGCTTTCGCCCTGGATCACGCTCTAGCT |
| | | CATGGGCTGCGCCGGGTGATCTACGTGATTCCCTTCACTAGCATCGTCGAGCAG |
| | | AACGCTGCGGTATTCCGTCGTGCACTCGGGGCCTTAGGCGAAGAGGCGGTGCT |
| | | GGAGCATCACAGCGCCTTCGTTGATGACCGCCGGCAGAGCCTGGAGGCCAAGA |
| | | AGAAACTGAACCTAGCGATGGAGAACTGGGACGCGCCTATCGTGGTGACCACT |
| | | GCAGTGCAGTTCTTCGAAAGCCTGTTTGCCGACCGTCCAGCCCAGTGCCGCAAG |
| | | CTACACAACATCGCCGGCAGCGTGGTGATTCTTGACGAGGCACAGACCCTACC |
| | | GCTCAAGCTGTTGCGGCCCTGCGTTGCCGCCCTTGATGAACTGGCGCTCAACTA |
| | | CCGTTGTAGCCCAGTTCTCTGTACTGCCACGCAGCCAGCGCTTCAATCGCCGGA |
| | | TTTCATCGGTGGGCTGCAGGACGTACGTGAGCTGGCGCCCGAGCCGCAGCGGC |
| | | TGTTCCGGGAGTTGGTGCGGGTACGAATACGGACATTGGGCCCGCTCGAAGAT |
| | | GCGGCCTTGACTGAGCAGATCGCCAGGCGTGAACAAGTGCTGTGCATCGTCAA |
| | | CAATCGACGCCAGGCCCGTGCGCTCTATGAGTCGCTTGCCGAGTTGCCCGGTGC |
| | | CCGCCATCTCACCACCCTGATGTGCGCCAAGCACCGTAGCAGCGTGCTGGCCGA |
| | | GGTGCGCCAGATGCTCAAAAAGGGGGAGCCCTGTCGCCTGGTGGCCACCTCGC |
| | | TGATCGAGGCCGGTGTGGATGTGGATTTTCCCGTGGTACTGCGTGCCGAGGCTG |
| | | GATTGGATTCCATCGCCCAGGCCGCGGGACGCTGCAATCGCGAAGGCAAGCGG |
| | | CCGCTGGCCGAAAGCGAGGTGCTGGTGTTCGCCGCGGCCAATTCTGACTGGGC |
| | | GCCACCCGAGGAACTCAAGCAGTTCGCCCAGGCCGCCCGCGAAGTGATGCGCC |
| | | TGCACCCGGATGATTGCCTGTCCATGGCGGCCATCGAGCGGTATTTTCGCATAC |
| | | TGTACTGGCAGAAGGGCGCGGAGGAGTTGGATGCGGGTAACCTGCTCGGCCTG |
| | | ATTGAGAGAGGCCGGCTCGATGGCCTGCCCTACGAGACTTTGGCCACCAAGTTC |
| | | CGCATGATCGACAGCCTTCAACTGCCGGTGATCATCCCATTTGATGACGAGGCC |
| | | AGAGCAGCCCTGCGCGAGCTGGAGTTCGCCGACGGCTGCGCCGCCATCGCCCG |
| | | TCGCCTGCAGCCATATCTGGTGCAGATGCCACGCAAGGGTTATCAGGCATTGCG |
| | | GGAAGCCGGTGCGATCCAGGCGGCGGCAGGTACGCGTTATGGTGAGCAGTTTA |
| | | TGGCGTTGGTCAACCCTGATCTGTATCACCACCAATTCGGGTTGCACTGGGATA |
| | | ATCCGGCCTTTGTCAGCAGCGAGCGGCTATGTTGGTAG |
| Cas5c | 48 | ATGGCCTACGGAATTCGCTTAATGGTCTGGGGCGAGCGTGCCTGCTTCACCCGC |
| | | CCGGAAATGAAGGTGGAACGCGTCTCTTACGATGCGATCACGCCGTCCGCCGC |
| | | GCGCGGCATTCTCGAGGCTATCCACTGGAAGCCGGCGATTCGCTGGGTGGTGG |
| | | ATCGCATTCAAGTGCTTAAGCCGATCCGCTTCGAATCCATCCGGCGCAACGAGG |
| | | TCGGCGGCAAGCTGTCCGCTGTCAGCGTCGGTAAGGCAATGAAGGCCGGGCGT |
| | | ACTAATGGTCTGGTGAATCTGGTCGAGGAGGATCGCCAGCAGCGCGCGACTAC |
| | | TCTGCTGCGCGATGTCTCCTATGTCATCGAGGCGCATTTCGAGATGACTGACAG |
| | | GGCTGGCGCCGACGATACGGTGGGCAAGCATCTGGATATCTTCAACCGTCGCG |
| | | CACGGAAGGGGCAGTGCTTCCATACACCCTGCCTAGGCGTGCGCGAGTTTCCGG |
| | | CCAGTTTTCGGTTGCTGGAAGAGGGCAGTGCCGAGCCTGAAGTCGATGCCTTTC |
| | | TGCGCGGCGAGCGTGATCTGGGCTGGATGCTGCATGACATTGACTTCGCCGATG |
| | | GCATGACCCCGCACTTCTTCCGTGCCCTGATGCGCGATGGGCTGATCGAGGTGC |
| | | CGGCCTTCAGGGCGGCAGAGGACAAGGCATGA |
| Cas8c | 49 | ATGATCCTTTCGGCCCTCAATGACTATTATCAGCGACTGCTGGAGCGGGGTGAA |
| | | GCGAATATCTCACCCTTCGGCTACAGCCAAGAAAAGATCAGTTACGCCCTGCTG |
| | | CTGTCCGCACAAGGAGAGTTGCTGGACGTGCAGGACATTCGCTTGCTCTCTGGC |
| | | AAGAAGCCTCAACCCAGGCTTATGAGTGTGCCGCAGCCGGAGAAGCGCACCTC |
| | | GGGCATCAAGTCCAACGTACTGTGGGACAAGACCAGCTATGTGCTGGGTGTTA |
| | | GTGCCAAGGGCGGAGAGCGTACTCAGCAGGAGCACGAGTCCTTCAAGACGCTG |
| | | CACCGGCAGATCTTGGTTGGGGAAGGCGACCCCGGTCTGCAGGCCTTGCTCCAG |
| | | TTCCTCGACTGTTGGCAGCCGGAGCAGTTCAAGCCCCCGCTGTTCAGCGAAGCA |
| | | ATGCTCGACAGCAACTTAGTGTTCCGCCTAGACGGCCAACAACGCTATCTGCAC |
| | | GAGACTCCGGCGGCCCTGGCGTTGCGTACCCGGCTGTTGGCCGACGGCGACAG |
| | | CCGCGAGGGGCTGTGCCTAGTCTGCGGCCAACGTCAGCCGTTGGCGCGCCTGCA |
| | | TCCAGCGGTCAAGGGCGTCAATGGTGCCCAGAGTTCGGGGGCTTCCATCGTCTC |
| | | CTTCAACCTCGACGCTTTTTCCTCCTACGGCAAGAGCCAGGGGGAAAATGCTCC |
| | | GGTCTCCGAACAGGCCGCCTTTGCCTACACCACGGTGCTCAACCATTTGTTGCG |
| | | TCGCGACGAGCACAACCGCCAGCGCCTGCAGATTGGCGACGCGAGTGTGGTGT |
| | | TCTGGGCGCAGGCGGATACTCCTGCTCAGGTGGCCGCCGCCGAGTCGACCTTCT |
| | | GGAACCTGCTGGAGCCACCCGCAGATGATGGTCAGGAAGCGGAAAAGCTGCGC |
| | | GGCGTGCTGGATGCTGTGGCCACGGGGCGGCCCTTGCATGAGCTCGACTCGCTA |
| | | ATGGAGGAAGGTACCCGCATTTTTGTGTTAGGGCTGGCGCCCAATACCTCGCGA |
| | | CTGTCCATTCGGTTCTGGGCAGTCGATAGCCTTGCGGTATTCACCCAGCATCTG |
| | | GCCGAGCATTTCCGGGATATGCACCTTGAGCCTCTGCCCTGGAAGACGGAGCCG |
| | | GCCATCTGGCGCTTGCTCTATGCTACCGCGCCCAGTCGTGACGGCAGAGCCAAG |
| | | ACCGAAGACGTACTCCCACAACTGGCCGGTGAAATGACCCGCGCCATCCTGAC |
| | | CGGCAGCCGCTATCCGCGCAGTTTGCTAGCCAACCTGATCATGCGCATGCGTGC |
| | | CGACGGCGACGTCTCTGGCATACGCGTCGCGCTGTGCAAGGCCGTGCTCGCTCG |
| | | CGAGGCACGCCTGAGCGGCAAAATTCACCAAGAGGAGCTACCTATGAGTCTCG |
| | | ACAAGGACGCCAGCAACCCCGGCTATCGCTTGGGGAGGCTGTTCGCCGTGTTG |
| | | GAAGGCGCCCAGCGCGCAGCCCTGGGCGACAGGGTCAATGCCACTATCCGTGA |
| | | CCGCTACTACGGTGCCGCGTCCAGCACGCCAGCCACGGTTTTCCCGATACTGCT |
| | | GCGCAACACACAAAACCACTTGGCCAAGCTGCGCAAGGAGAAGCCCGGACTAG |
| | | CAGTGAACCTAGAGCGCGATATAGGCGAAATCATTGACGGTATGCAGAGCCAA |
| | | TTCCCGCGTTGCCTGCGCCTGGAGGACCAGGGACGCTTTGCTATTGGTTACTAC |
| | | CAACAGGCCCAGGCCCGTTTCAACCGTGGCCCCGATTCCGTCGAGTAA |
| Cas7c | 50 | ATGACCGCCATCTCCAACCGCTACGAGTTCGTTTACCTCTTTGATGTCAGCAAT |
| | | GGCAATCCCAATGGCGACCCGGATGCTGGCAACATGCCGCGTCTCGATCCGGA |
| | | AACCAACCAGGGGTTGGTCACTGACGTTTGCCTCAAGCGCAAGATCCGCAACT |
| | | ACGTCAGCCTGGAGCAGGAAAGTGCCCCCGGCTATGCCATCTATATGCAGGAA |
| | | AAATCCGTGCTGAATAACCAGCACAAACAGGCCTACGAGGCGCTCGGTATCGA |
| | | GTCAGAGGCAAAGAAACTGCCCAAGGACGAAGCCAAGGCGCGCGAACTGACC |
| | | TCTTGGATGTGCAAGAACTTCTTCGATGTGCGTGCTTTCGGGGCGGTGATGACC |
| | | ACCGAGATTAATGCCGGCCAGGTGCGTGGACCGATCCAACTGGCATTCGCCAC |
| | | GTCTATCGACCCGGTATTGCCTATGGAGGTATCCATCACCCGCATGGCGGTGAC |
| | | TAACGAAAAGGATTTGGAGAAGGAACGCACCATGGGACGCAAGCACATCGTGC |
| | | CTTACGGCTTGTACCGCGCCCATGGTTTCATCTCTGCCAAGTTGGCCGAGCGAA |
| | | CCGGCTTTTCCGACGACGACTTGGAACTGCTATGGCGCGCTTTGGCCAATATGT |
| | | TCGAACACGACCGCTCGGCGGCACGTGGCGAGATGGCAGCGCGCAAGTTGATC |
| | | GTCTTCAAGCATGAGCATGCCATGGGCAATGCACCCGCCCATGTGCTGTTCGGC |
| | | AGCGTTAAGGTCGAGCGAGTCGAGGGGGACGCAGTTACACCAGCACGCGGTTT |
| | | CCAGGATTACCGTGTCAGCATCGATGCGGAAGCTCTGCCTCAGGGCGTGAGCGT |
| | | GCGCGAGTACCTCTAG |
| Cas3 | 51 | MDAEASDTHFFAHSTLKADRSDWQPLVEHLQAVARLAGEKAAFFGGGELAALAG |
| | | LLHDLGKYTDEFQRRIAGDAIRVDHSTRGAILAVERYGALGQLLAYGIAGHHAGLA |
| | | NGREAGERTALVDRLKGVGLPRLLEGWCVEIVLPERLQPPPLKARLERGFFQLAFL |
| | | GRMLFSCLVDADYLDTEAFYHRVEGRRSLREQARPTLAELRAALDRHLTEFKGDT |
| | | PVNRVRGEILAGVRGKASELPGLFSLTVPTGGGKTLASLAFALDHALAHGLRRVIY |
| | | VIPFTSIVEQNAAVFRRALGALGEEAVLEHHSAFVDDRRQSLEAKKKLNLAMENW |
| | | DAPIVVTTAVQFFESLFADRPAQCRKLHNIAGSVVILDEAQTLPLKLLRPCVAALDE |
| | | LALNYRCSPVLCTATQPALQSPDFIGGLQDVRELAPEPQRLFRELVRVRIRTLGPLE |
| | | DAALTEQIARREQVLCIVNNRRQARALYESLAELPGARHLTTLMCAKHRSSVLAEV |
| | | RQMLKKGEPCRLVATSLIEAGVDVDFPVVLRAEAGLDSIAQAAGRCNREGKRPLA |
| | | ESEVLVFAAANSDWAPPEELKQFAQAAREVMRLHPDDCLSMAAIERYFRILYWQK |
| | | GAEELDAGNLLGLIERGRLDGLPYETLATKFRMIDSLQLPVIIPFDDEARAALRELEF |
| | | ADGCAAIARRLQPYLVQMPRKGYQALREAGAIQAAAGTRYGEQFMALVNPDLYH |
| | | HQFGLHWDNPAFVSSERLCW* |
| Cas5c | 52 | MAYGIRLMVWGERACFTRPEMKVERVSYDAITPSAARGILEAIHWKPAIRWVVDRI |
| | | QVLKPIRFESIRRNEVGGKLSAVSVGKAMKAGRINGLVNLVEEDRQQRATTLLRD |
| | | VSYVIEAHFEMTDRAGADDTVGKHLDIFNRRARKGQCFHTPCLGVREFPASFRLLE |
| | | EGSAEPEVDAFLRGERDLGWMLHDIDFADGMTPHFFRALMRDGLIEVPAFRAAED |
| | | KA* |
| Cas8c | 53 | MILSALNDYYQRLLERGEANISPFGYSQEKISYALLLSAQGELLDVQDIRLLSGKKP |
| | | QPRLMSVPQPEKRTSGIKSNVLWDKTSYVLGVSAKGGERTQQEHESFKTLHRQILV |
| | | GEGDPGLQALLQFLDCWQPEQFKPPLFSEAMLDSNLVFRLDGQQRYLHETPAALA |
| | | LRTRLLADGDSREGLCLVCGQRQPLARLHPAVKGVNGAQSSGASIVSFNLDAFSSY |
| | | GKSQGENAPVSEQAAFAYTTVLNHLLRRDEHNRQRLQIGDASVVFWAQADTPAQ |
| | | VAAAESTFWNLLEPPADDGQEAEKLRGVLDAVATGRPLHELDSLMEEGTRIFVLG |
| | | LAPNTSRLSIRFWAVDSLAVFTQHLAEHFRDMHLEPLPWKTEPAIWRLLYATAPSR |
| | | DGRAKTEDVLPQLAGEMTRAILTGSRYPRSLLANLIMRMRADGDVSGIRVALCKA |
| | | VLAREARLSGKIHQEELPMSLDKDASNPGYRLGRLFAVLEGAQRAALGDRVNATI |
| | | RDRYYGAASSTPATVFPILLRNTQNHLAKLRKEKPGLAVNLERDIGEIIDGMQSQFP |
| | | RCLRLEDQGRFAIGYYQQAQARFNRGPDSVE* |
| Cas7c | 54 | MTAISNRYEFVYLFDVSNGNPNGDPDAGNMPRLDPETNQGLVTDVCLKRKIRNYV |
| | | SLEQESAPGYAIYMQEKSVLNNQHKQAYEALGIESEAKKLPKDEAKARELTSWMC |
| | | KNFFDVRAFGAVMTTEINAGQVRGPIQLAFATSIDPVLPMEVSITRMAVTNEKDLE |
| | | KERTMGRKHIVPYGLYRAHGFISAKLAERTGFSDDDLELLWRALANMFEHDRSAA |
| | | RGEMAARKLIVFKHEHAMGNAPAHVLFGSVKVERVEGDAVTPARGFQDYRVSID |
| | | AEALPQGVSVREYL* |

While preferred embodiments of the present disclosures have been shown and described herein, it will be obvious to those skilled in the art that such embodiments are provided by way of example only. Numerous variations, changes, and substitutions will now occur to those skilled in the art without departing from the disclosures. It should be understood that various alternatives to the embodiments of the disclosures described herein may be employed in practicing the disclosures. It is intended that the following claims define the scope of the disclosures and that methods and structures within the scope of these claims and their equivalents be covered thereby.

What is claimed is:

1. A nucleic acid sequence having at least 80% identity to SEQ ID NO: 39.

2. The nucleic acid of claim 1, comprising SEQ ID NO: 25.

3. The nucleic acid of claim 1 or claim 2, comprising SEQ ID NO: 24.

4. The nucleic acid of any one of claims 1-3, comprising SEQ ID NO: 12.

5. The nucleic acid of any one of claims 1-4, comprising SEQ ID NO: 13.

6. The nucleic acid of claim 1, comprising SEQ ID NO: 39, or a sequence at least 90% identical to SEQ ID NO: 39.

7. A CRISPR array comprising the nucleic acid sequence of any one of claims 1-6.

8. The CRISPR array of claim 7, further comprising a promoter.

9. The CRISPR array of claim 8, wherein the promoter comprises a sequence at least about 80%, identical to any one of SEQ ID NOS: 11, 1-10 or 19.

10. The CRISPR array of claim 9, wherein the promoter comprises a sequence at least about 80%, identical to SEQ ID NO: 11.

11. The CRISPR array of any one of claims 1-10, comprising a sequence at least 80% identical to SEQ ID NO: 43.

12. A CRISPR array comprising a sequence at least 80% identical to SEQ ID NO: 43.

13. A bacteriophage comprising the nucleic acid of any one of claims 2-6, or the CRISPR array of any one of claims 7-11.

14. A bacteriophage comprising the nucleic acid of claim 1 or the CRISPR array of claim 12.

15. The bacteriophage of claim 13 or claim 14, wherein the nucleic acid and/or CRISPR array replaces bacteriophage DNA.

16. The bacteriophage of claim 15, wherein the bacteriophage DNA is from a p004ke, p00c0, or p00ex phage.

17. The bacteriophage of any one of claims 13-16, wherein the nucleic acid and/or CRISPR array is part of a CRISPR system present in the bacteriophage.

18. The bacteriophage of claim 17, wherein the CRISPR system comprises a total of about 3000 nucleobases to about 8000 nucleobases.

19. The bacteriophage of claim 17 or claim 18, wherein the CRISPR system comprises a Type I CRISPR-Cas system, a Type II CRISPR-Cas system, a Type V CRISPR-Cas system, or a CRISPR-Cpf1 system.

20. A recombinant phage comprising at least 80% sequence identity to p004ke009.

21. A recombinant phage comprising at least 80% sequence identity to p00c0e030.

22. A recombinant phage comprising at least 80% sequence identity to p00exe014.

23. A recombinant phage comprising at least 90% sequence identity to p004ke009.

24. A recombinant phage comprising at least 90% sequence identity to p00c0e030.

25. A recombinant phage comprising at least 90% sequence identity to p00exe014.

26. A bacteriophage comprising a CRISPR system comprising:
(a) a CRISPR array comprising a first spacer sequence comprising a sequence selected from SEQ ID NOS: 12 or 20-37; and
(b) a nucleic acid sequence encoding a CRISPR associated nuclease.

27. The bacteriophage of claim 26, wherein the first spacer sequence comprises SEQ ID NO: 12, SEQ ID NO: 25, or SEQ ID NO: 24.

28. The bacteriophage of claim 26, wherein the CRISPR array comprises a second spacer sequence.

29. The bacteriophage of claim 28, wherein the first spacer sequence comprises SEQ ID NO: 12, and the second spacer sequence comprises SEQ ID NO: 25, or the first spacer sequence comprises SEQ ID NO: 12, and the second spacer sequence comprises SEQ ID NO: 24, or the first spacer sequence comprises SEQ ID NO: 25, and the second spacer sequence comprises SEQ ID NO: 24.

30. The bacteriophage of claim 28, wherein the CRISPR array comprises a third spacer sequence.

31. The bacteriophage of claim 30, wherein the first spacer sequence comprises SEQ ID NO: 12, the second spacer sequence comprises SEQ ID NO: 25, and the third spacer sequence comprises SEQ ID NO: 24.

32. A bacteriophage comprising a CRISPR system comprising:
(a) a CRISPR array comprising a first spacer sequence and a second spacer sequence, wherein the first spacer sequence is complementary to a first target nucleotide sequence in a first Escherichia species, and the second spacer sequence is complementary to a second target nucleotide sequence in the first Escherichia species and/or a second Escherichia species; optionally further comprising a third spacer sequence complementary to a third target nucleotide sequence in the Escherichia species, second Escherichia species, and/or a third Escherichia species; and
(b) a nucleic acid sequence encoding a CRISPR associated nuclease.

33. The bacteriophage of claim 32, wherein the first spacer sequence comprises a sequence at least about 90%, sequence identity to any one of SEQ ID NOS: 12 or 20-37, wherein the second spacer sequence comprises a sequence at least about 90%, sequence identity to any one of SEQ ID NOS: 12 or 20-37, and if present, the third spacer sequence comprises a sequence at least about 90%, sequence identity to any one of SEQ ID NOS: 12 or 20-37.

34. The bacteriophage of any one of claims 26-33, wherein the CRISPR array comprises a repeat sequence comprising a sequence with at least about 90% sequence identity to any one of SEQ ID NOs: 13-18.

35. The bacteriophage of any one of claims 26-34, wherein the CRISPR array comprises a promoter sequence comprising a sequence at least about 90% identical to any one of SEQ ID NOs: 11, 1-10 or 19.

36. The bacteriophage of any one of claims 26-35, wherein the CRISPR system comprises a Type I CRISPR-Cas system, a CRISPR-Cpf1 system, a Type II CRISPR-Cas system, or a Type V CRISPR-Cas system.

37. A bacteriophage modified to replace bacteriophage DNA with a nucleic acid sequence encoding a CRISPR system comprising: a CRISPR array comprising a spacer sequence complementary to a target nucleotide sequence in a target bacteria, and a sequence encoding a CRISPR nuclease, wherein the target bacteria comprises an Escherichia species.

38. The bacteriophage of claim 37, wherein the CRISPR system comprises a total of about 3000 nucleobases to about 8000 nucleobases.

39. The bacteriophage of claim 37 or claim 38, wherein the bacteriophage DNA is not essential for viability or functionality of the bacteriophage.

40. The bacteriophage of any one of claims 37-39, wherein the bacteriophage DNA is from a p004ke, p00c0, or p00ex phage.

41. The bacteriophage of any one of claims 37-40, wherein the CRISPR array comprises a spacer sequence comprising a sequence having at least about 90% sequence identity to any one of SEQ ID NOS: 12 or 20-37.

42. The bacteriophage of any one of claims 37-41, wherein the CRISPR array comprises a repeat sequence comprising at least about 90% identity to any one of SEQ ID NOs: 13-18.

43. The bacteriophage of any one of claims 37-42, wherein the CRISPR array comprises a promoter sequence comprises at least about 90% identity to any one of SEQ ID NOs: 11, 1-10 or 19.

44. The bacteriophage of any one of claims 37-43, wherein the CRISPR system comprises a Type I CRISPR-Cas system, a CRISPR-Cpf1 system, a Type II CRISPR-Cas system, or a Type V CRISPR-Cas system.

45. A composition comprising the bacteriophage of any one of claims 1-44, and a wild-type phage.

46. The composition of claim 45, wherein the wild-type phage comprises p00ke.

47. The composition of claim 46, wherein the wild-type phage comprises p5516.

48. The composition of claim 47, wherein the wild-type phage comprises p00jc.

49. A composition comprising a first phage having at least 80% sequence identity to p004ke009, and a second phage having at least 80% sequence identity to p00c0e030, p00exe014, p00jc, p00ke, or p5516.

50. A composition comprising a first phage having at least 80% sequence identity to p00c0e030, and a second phage having at least 80% sequence identity to p004ke009, p00exe014, p00jc, p00ke, or p5516.

51. A composition comprising a first phage having at least 80% sequence identity to p00exe014, and a second phage having at least 80% sequence identity to p004ke009, p00c0e030, p00jc, p00ke, or p5516.

52. A composition comprising a first phage having at least 80% sequence identity to p00jc, and a second phage having at least 80% sequence identity to p004ke009, p00c0e030, p00exe014, p00ke, or p5516.

53. A composition comprising a first phage having at least 80% sequence identity to p00ke, and a second phage having at least 80% sequence identity to p004ke009, p00c0e030, p00exe014, p00jc, or p5516.

54. A composition comprising a first phage having at least 80% sequence identity to p5516, and a second phage having at least 80% sequence identity to p004ke009, p00c0e030, p00exe014, p00jc, or p00ke.

55. A pharmaceutical composition comprising:
(a) the bacteriophage of any one of claims 1-44 or the composition of any one of claims 45-54; and
(b) a pharmaceutically acceptable excipient.

56. The pharmaceutical composition of claim 55, wherein the pharmaceutical composition is in the form of a tablet, a capsule, a liquid, a syrup, an oral formulation, an intravenous formulation, an intranasal formulation, an ocular formulation, an otic formulation, a subcutaneous formulation, a topical formulation, a transdermal formulation, a transmucosal formulation, an inhalable respiratory formulation, a suppository, a lyophilized formulation, a nebulizable formulation, and any combination thereof.

57. A method of killing bacteria comprising introducing into the bacteria genetic material from the bacteriophage of any one of claims 1-44 or the composition of any one of claims 45-56.

58. A method of treating a disease in an individual in need thereof, the method comprising administering to the individual the bacteriophage of any one of claims 1-44 or the composition of any one of claims 45-56.

59. A method of killing bacteria comprising contacting the bacteria with a composition comprising a bacteriophage at least 80% identity to p00jc.

60. A method of killing bacteria comprising contacting the bacteria with a composition comprising a bacteriophage at least 80% identity to p00ke.

61. A method of killing bacteria comprising contacting the bacteria with a composition comprising a bacteriophage at least 80% identity to p5516.

62. A method of treating a disease in an individual in need thereof, the method comprising administering to the individual a composition comprising a bacteriophage at least 80% identity to p00jc.

63. A method of treating a disease in an individual in need thereof, the method comprising administering to the individual a composition comprising a bacteriophage at least 80% identity to p00ke.

64. A method of treating a disease in an individual in need thereof, the method comprising administering to the individual a composition comprising a bacteriophage at least 80% identity to p5516.

65. A method of killing a plurality of bacteria, the method comprising combining the plurality of bacteria with a first bacteriophage and a second bacteriophage, wherein the first bacteriophage targets a first subset of the plurality of bacteria, and the second bacteriophage targets a second subset of the plurality of bacteria, wherein the plurality of bacteria comprises two or more bacteria of Table 6.

66. A method of treating a disease in an individual comprising a plurality of bacteria, the method comprising administering to the individual a first bacteriophage and a second bacteriophage, wherein the first bacteriophage targets a first subset of the plurality of bacteria, and a second bacteriophage targets the second subset of the plurality of bacteria, wherein the plurality of bacteria comprises two more bacteria of Table 6.

67. The method of claim 65 or claim 66, wherein the plurality of bacteria comprises at least 100, 150, 200, 250, 300, or 350 bacteria of Table 6.

68. The method of any one of claims 65-67, wherein the first bacteriophage comprises the bacteriophage of any one of claims 1-44.

69. The method of any one of claims 65-68, wherein the second bacteriophage comprises the bacteriophage of any one of claims 1-44.

70. The method of claim 65 or claim 66, wherein: (i) the first bacteriophage comprises a phage at least 80% identical to p00jc, (ii) the first bacteriophage comprises a phage at least 80% identical to p00ke, (iii) the first bacteriophage comprises a phage at least 80% identical to p5516, (iv) the first bacteriophage comprises a phage at least 80% identical to p004Ke009, (v) the first bacteriophage comprises a phage at least 80% identical to p00c0e030, or (vi) the first bacteriophage comprises a phage at least 80% identical to p00exe014.

71. The method of claim 65 or claim 66, wherein (i) the first phage has at least 80% sequence identity to p004ke009, and the second phage has at least 80% sequence identity to p00c0e030, p00exe014, p00jc, p00ke, or p5516, (ii) the first phage has at least 80% sequence identity to p00c0e030, and the second phage has at least 80% sequence identity to p004ke009, p00exe014, p00jc, p00ke, or p5516, (iii) the first phage has at least 80% sequence identity to p00exe014, and the second phage has at least 80% sequence identity to p004ke009, p00c0e030, p00jc, p00ke, or p5516, (iv) the first phage has at least 80% sequence identity to p00jc, and the second phage has at least 80% sequence identity to p004ke009, p00c0e030, p00exe014, p00ke, or p5516, (v) the first phage has at least 80% sequence identity to p00ke, and the second phage has at least 80% sequence identity to p004ke009, p00c0e030, p00exe014, p00jc, or p5516, or (vi) the first phage has at least 80% sequence identity to p5516, and the second phage has at least 80% sequence identity to p004ke009, p00c0e030, p00exe014, p00jc, or p00ke.


SEQUENCE LISTING (56 sequences)

SEQ ID NO: 1 - DNA, 97 bp, Unknown
  Description of Unknown Phage genome sequence
acaagcggcacattgtgcctattgcgaattaggcacaatgtgcctaatctaacgtcatgccagccacaacggcgaggcgccaagaaggatagaagcc

SEQ ID NO: 2 - DNA, 35 bp, Artificial Sequence
  Description of Artificial Sequence Synthetic oligonucleotide
tttacagctagctcagtcctagggactgtgctagc

SEQ ID NO: 3 - DNA, 155 bp, Pseudomonas aeruginosa
gatttttttcgggtgaggttgcgggctgttcggtaggtttataaacactgctatccaaagctatggacacgctcggctacgagaacagttggcgtgatggcctctagcaattagattgttatgcgacatccgcagacttggcagggagcgcacct

SEQ ID NO: 4 - DNA, 35 bp, Artificial Sequence
  Description of Artificial Sequence Synthetic oligonucleotide
tttacggctagctcagtcctaggtatagtgctagc

SEQ ID NO: 5 - DNA, 199 bp, Pseudomonas aeruginosa
atccgagggatacgggccttgtcagcacggtgttgctaatgagagcctttgcccgggcaatagtacgggcagtgtgtagcggattgaaagacgctgaatcactgacaggcatgaagactatcgatagagtctgatagtgtcgccgccgcacagcggatagagtccacagtcattgaagtgttaatccgcgatcaagctc

SEQ ID NO: 6 - DNA, 216 bp, Unknown
  Description of Unknown Phage genome sequence
gacctagcttttatagcgggtttcgtggtttatagcccattgaaaaaaatctcacatctatatcacaggtgtgcactcgttcccgaaaggttctgagtctacttgatcaagtattgaaataccatcgtaaaggaaaaagacatgtctattcgtgatagcgaaaacaacaacggccaacagcagcagaccgcgcaaactgccgcccccgccccgcaa

SEQ ID NO: 7 - DNA, 182 bp, Unknown
  Description of Unknown Phage genome sequence
ttcaatttaagtagtaacgaggtcagcccggaatctttgggtattcttaaggtatttctgactcagtgtggttgggacagcttcactgtacattgcactggatttgttaatttcttataccggggcaccatgggcagcaaatcgtgttacgaattccgtctaaccaataagcgagctaaata

SEQ ID NO: 8 - DNA, 105 bp, Artificial Sequence
  Description of Artificial Sequence Synthetic polynucleotide
cgcggaacccctatttgtttatttttctaaatacattcaaatatgtatccgctcatgagacaataaccctgataaatgcttcaataatattgaaaaaggaagagt

SEQ ID NO: 9 - DNA, 161 bp, Pseudomonas aeruginosa
cttcaagaattcgtattgaccccatagacagcttcgtcgacgcccgtcccggcccccttgggcttgccggacggcttatgtcatgatggcgccaccctcgcaggttcaaggccggctttcttcctctatgaacaaatcccttgcgctgactacgtaatcac

SEQ ID NO: 10 - DNA, 207 bp, Pseudomonas aeruginosa
cttcaagaattcggggtattcctgatcctgcgccgctagcgccgcgcacggccactaggcccgcgccgatagccagtcgcgctcccggctggcacactactcccatttccgccggaaacgcgcgcaacgtaccggcaacgaacgtggaaagaccatgaaagactggctggatgagattcactggaacgccgtgacctacgtatgcac

SEQ ID NO: 11 - DNA, 59 bp, Escherichia coli
gaaaattattttaaatttcctctagtcaggccggaataactccctataatgcgacacca

SEQ ID NO: 12 - DNA, 34 bp, Artificial Sequence
  Description of Artificial Sequence Synthetic oligonucleotide
atttatcacaaaaggattgttcgatgtccaacaa

SEQ ID NO: 13 - DNA, 32 bp, Artificial Sequence
  Description of Artificial Sequence Synthetic oligonucleotide
gtcgcgccccgcacgggcgcgtggattgaaac

SEQ ID NO: 14 - DNA, 32 bp, Artificial Sequence
  Description of Artificial Sequence Synthetic oligonucleotide
gtcgcgccccgcacgggcgcgtggagtgaaag

SEQ ID NO: 15 - DNA, 32 bp, Artificial Sequence
  Description of Artificial Sequence Synthetic oligonucleotide
gtcgcgccccgcacgggtgcgtggattgaaac

SEQ ID NO: 16 - DNA, 32 bp, Artificial Sequence
  Description of Artificial Sequence Synthetic oligonucleotide
gtcgcgccccgcatgggcgcgtggattgaaca

SEQ ID NO: 17 - DNA, 32 bp, Artificial Sequence
  Description of Artificial Sequence Synthetic oligonucleotide
gtcgcgccctacgcgggcgcgtggagtgaaag

SEQ ID NO: 18 - DNA, 36 bp, Artificial Sequence
  Description of Artificial Sequence Synthetic oligonucleotide
gtctaagaactttaaataatttctactgttgtagat

SEQ ID NO: 19 - DNA, 53 bp, Artificial Sequence
  Description of Artificial Sequence Synthetic oligonucleotide
tttacagctagctcagtcctagggactgtgctagcattaaagaggagaaaatg

SEQ ID NO: 20 - DNA, 34 bp, Artificial Sequence
  Description of Artificial Sequence Synthetic oligonucleotide
cggcagatggtcattgacctcggcataggtcaga

SEQ ID NO: 21 - DNA, 34 bp, Artificial Sequence
  Description of Artificial Sequence Synthetic oligonucleotide
ataatcgatcatttgacgactcctgtcttagcgt

SEQ ID NO: 22 - DNA, 34 bp, Artificial Sequence
  Description of Artificial Sequence Synthetic oligonucleotide
tgacatttcagcttttgtaagcgccataggttca

SEQ ID NO: 23 - DNA, 34 bp, Artificial Sequence
  Description of Artificial Sequence Synthetic oligonucleotide
gcctttgtgctgcagtttattgagctgccgttcc

SEQ ID NO: 24 - DNA, 34 bp, Artificial Sequence
  Description of Artificial Sequence Synthetic oligonucleotide
atgccggatgcggcgtgaacgccttatccggcct

SEQ ID NO: 25 - DNA, 34 bp, Artificial Sequence
  Description of Artificial Sequence Synthetic oligonucleotide
aaccgtctgtactgaagcgcaaccgttctcacgg

SEQ ID NO: 26 - DNA, 34 bp, Artificial Sequence
  Description of Artificial Sequence Synthetic oligonucleotide
gtaagattaaaaaactgatcgagctggttgaaga

SEQ ID NO: 27 - DNA, 34 bp, Artificial Sequence
  Description of Artificial Sequence Synthetic oligonucleotide
ttatgactcctccagtatcaaagtcctgaaaggg

SEQ ID NO: 28 - DNA, 34 bp, Artificial Sequence
  Description of Artificial Sequence Synthetic oligonucleotide
gtaaccgcggtctgcactctgcattcactgttcg

SEQ ID NO: 29 - DNA, 34 bp, Artificial Sequence
  Description of Artificial Sequence Synthetic oligonucleotide
cctatccttctggtcgactacacatgggccacgt

SEQ ID NO: 30 - DNA, 34 bp, Artificial Sequence
  Description of Artificial Sequence Synthetic oligonucleotide
tgatctgtctgcggacattaacgaacacctgatc

SEQ ID NO: 31 - DNA, 34 bp, Artificial Sequence
  Description of Artificial Sequence Synthetic oligonucleotide
tgtcatgcaagtccctcgggtcgagaagatcacc

SEQ ID NO: 32 - DNA, 34 bp, Artificial Sequence
  Description of Artificial Sequence Synthetic oligonucleotide
caatatcatggtcgtgtccaggcactggcagatg

SEQ ID NO: 33 - DNA, 34 bp, Artificial Sequence
  Description of Artificial Sequence Synthetic oligonucleotide
tatgaaaaaccgactaccgaacgtaagcgcgcta

SEQ ID NO: 34 - DNA, 34 bp, Artificial Sequence
  Description of Artificial Sequence Synthetic oligonucleotide
tgaacaacgaaatcatcctggtaacctgtggttc

SEQ ID NO: 35 - DNA, 25 bp, Artificial Sequence
  Description of Artificial Sequence Synthetic oligonucleotide
cattggtagccatgtttctttcctg

SEQ ID NO: 36 - DNA, 25 bp, Artificial Sequence
  Description of Artificial Sequence Synthetic oligonucleotide
atactggaggagtcataagaattcg

SEQ ID NO: 37 - DNA, 25 bp, Artificial Sequence
  Description of Artificial Sequence Synthetic oligonucleotide
cggcgaccagtgccgtagtattgat

SEQ ID NO: 38 - DNA, 98 bp, Artificial Sequence
  Description of Artificial Sequence Synthetic oligonucleotide
gtcgcgccccgcacgggcgcgtggattgaaacatttatcacaaaaggattgttcgatgtccaacaagtcgcgccccgcacgggcgcgtggattgaaac

SEQ ID NO: 39 - DNA, 230 bp, Artificial Sequence
  Description of Artificial Sequence Synthetic polynucleotide
gtcgcgccccgcacgggcgcgtggattgaaacatttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaacatgccggatgcggcgtgaacgccttatccggcctgtcgcgccccgcacgggcgcgtggattgaaac

SEQ ID NO: 40 - DNA, 166 bp, Artificial Sequence
  Description of Artificial Sequence Synthetic polynucleotide
atttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaacatgccggatgcggcgtgaacgccttatccggcct

SEQ ID NO: 41 - DNA, 219 bp, Artificial Sequence
  Description of Artificial Sequence Synthetic polynucleotide
gtctaagaactttaaataatttctactgttgtagatcattggtagccatgtttctttcctggtctaagaactttaaataatttctactgttgtagatatactggaggagtcataagaattcggtctaagaactttaaataatttctactgttgtagatcggcgaccagtgccgtagtattgatgtctaagaactttaaataatttctactgttgtagat

SEQ ID NO: 42 - DNA, 157 bp, Artificial Sequence
  Description of Artificial Sequence Synthetic polynucleotide
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaac

SEQ ID NO: 43 - DNA, 289 bp, Artificial Sequence
  Description of Artificial Sequence Synthetic polynucleotide
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaacatgccggatgcggcgtgaacgccttatccggcctgtcgcgccccgcacgggcgcgtggattgaaac

SEQ ID NO: 44 - DNA, 278 bp, Artificial Sequence
  Description of Artificial Sequence Synthetic polynucleotide
gaaaattattttaaatttcctctagtcaggccggaataactccctataatgcgacaccagtctaagaactttaaataatttctactgttgtagatcattggtagccatgtttctttcctggtctaagaactttaaataatttctactgttgtagatatactggaggagtcataagaattcggtctaagaactttaaataatttctactgttgtagatcggcgaccagtgccgtagtattgatgtctaagaactttaaataatttctactgttgtagat

SEQ ID NO: 45 - DNA, 289 bp, Artificial Sequence
  Description of Artificial Sequence Synthetic polynucleotide
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaacaaccgtctgtactgaagcgcaaccgttctcacgggtcgcgccccgcacgggcgcgtggattgaaactgaacaacgaaatcatcctggtaacctgtggttcgtcgcgccccgcacgggcgcgtggattgaaac

SEQ ID NO: 46 - DNA, 3900 bp, Artificial Sequence
  Description of Artificial Sequence Synthetic polynucleotide
agtatctatcaggaattcgtaaacaaatattccctgtcgaaaacgctgcgctttgaattaattccacaaggcaaaacgctggaaaacatcaaagcgcgtggccttatcctggatgacgaaaagcgtgcaaaagactacaagaaagctaaacaaatcatcgacaaatatcatcagttttttattgaagaaattcttagctccgtgtgtatcagtgaagaccttcttcagaactatagcgatgtttacttcaagctgaagaaatcggacgatgataatctgcagaaagattttaagagtgccaaggatactatcaagaaacagattagtgagtacattaaggattccgagaagttcaaaaacctgttcaaccagaacctgatcgatgccaagaaagggcaggaatccgatctgatcctgtggcttaaacaatccaaagataacggcatcgaactgtttaaggcaaatagcgacatcacagatatcgatgaagcactggaaattatcaaatcgttcaaagggtggacaacctacttcaaaggatttcatgaaaatcgtaaaaacgtgtacagctcaaacgacatcccaacctctatcatttaccgcatcgttgacgataatttacccaaatttttggagaacaaagcgaaatatgaatctctgaaggataaggcgccggaagcgatcaattatgaacagatcaaaaaggacttggcggaggagttgacttttgatattgactataaaacgtcggaagtgaaccagcgcgtctttagcctggacgaagttttcgagattgcaaactttaataactacttaaatcaatccgggattactaagtttaatacgattattggcgggaaattcgttaacggtgaaaacaccaaacgtaaaggtatcaacgaatacattaatctgtatagccagcaaatcaacgataaaacactcaaaaaatataagatgtccgtgctgttcaaacagattttaagtgatacggaaagtaagtcatttgtgattgacaaactggaagatgactccgatgtagtgacgactatgcagtcattttatgaacagattgcggctttcaaaaccgtcgaagagaagtcgatcaaagagacgctcagcctcttatttgatgatctcaaagcccagaagctggatctttcgaaaatctactttaaaaacgataagtctttaaccgacctgtcccaacaggtgtttgatgactactccgtgattggtactgccgtgctggaatatattacccagcaaattgctccgaaaaacttagataacccaagtaagaaagaacaggaactgattgcgaaaaaaacggaaaaagcgaaatatctgagcctggaaaccattaaactggccctggaagaatttaacaaacatcgtgatatcgacaaacaatgccgtttcgaagaaatcctggccaattttgccgccattccgatgattttcgatgaaatcgcacagaataaggacaatctggcgcagatctcaattaaataccagaatcagggtaagaaagatctcctgcaggctagcgccgaagatgatgtgaaagcgatcaaagacctgctggaccaaactaataatctgctgcataaactgaagatctttcacattagccagtcagaagacaaggcgaacattctcgataaagatgaacacttctacctggttttcgaagaatgctacttcgaattagcaaacattgtccccctctataataaaattcgcaattatattactcagaaaccctatagcgatgaaaaattcaaactgaatttcgaaaactccaccctggcgaacggttgggataagaataaagagccagataataccgccattctgtttatcaaagacgataaatattatttaggcgtcatgaacaagaaaaacaacaaaatctttgacgacaaagccatcaaggaaaataagggtgaaggttacaagaaaatcgtttataaactgctgcctggggcgaacaaaatgctgccgaaagtgttttttagcgccaagagcattaaattttataatccgagtgaagacatcctgcgcatccgtaatcattcgacgcataccaaaaatggcagcccgcaaaaaggctatgaaaagtttgagttcaatattgaagattgtcgtaagtttatcgacttttacaaacaatcgatcagcaaacatcccgaatggaaagactttggcttccgtttctccgacacccaacgttataactcgatcgatgagttctatcgcgaggtcgaaaatcagggttataaactgacattcgaaaacatcagcgagtcatacattgactccgttgtaaatcagggcaaactgtacctcttccagatttataataaagatttcagcgcgtactcaaagggccgccccaatctccataccttatattggaaggcattgtttgatgagcgtaatctccaggatgtagtgtacaaactcaatggcgaggcggaactgttttatcgtaaacagagcattccaaagaaaattacccacccggcgaaggaagcgattgccaacaaaaacaaggacaatccgaaaaaggaaagcgtgttcgagtacgacctgattaaagacaaacgctttacggaagataagtttttctttcactgcccgatcacgattaactttaaatcgtccggcgcaaacaaatttaacgacgaaattaacttgctgctgaaagagaaagcgaacgatgtgcacattctgtcgatcgatcgtggtgaacgccatctggcgtattacacgctggtggatggcaagggtaacattatcaagcaggataccttcaatatcatcggtaatgatcgcatgaaaacgaattatcacgacaaactggctgctatcgagaaagatcgtgatagcgctcgcaaagactggaaaaaaatcaacaacatcaaggaaatgaaagaaggttatctttctcaggtagtacacgaaatcgccaaattagtcattgaatataatgctattgtagtgtttgaagatctgaactttggtttcaaacgcggtcgcttcaaggtcgaaaaacaggtttaccagaaattagagaaaatgttgattgagaaactgaactatctggtcttcaaggacaatgagtttgataagacaggtggcgttctgcgtgcgtatcagctgaccgccccgttcgaaaccttcaagaaaatgggtaaacagacgggtattatctattacgtgccggcaggcttcaccagcaagatttgcccggtcacgggcttcgtcaaccaactgtacccaaagtatgaaagtgtatccaagagccaggagttctttagcaagtttgataaaatctgctacaacctggataagggctatttcgagttcagcttcgactacaaaaacttcggcgacaaagccgcgaaaggcaagtggacgattgcctccttcggcagccgcctgattaactttcgcaactcggacaaaaatcataattgggatacccgcgaagtgtatcctacgaaagagctggagaaactgctcaaagactactcgatcgaatatggtcacggtgagtgtattaaagccgcaatttgcggtgaaagcgataaaaagtttttcgccaaactcaccagcgtattaaatactatccttcagatgcgcaattccaagacaggtaccgaactcgattatctgatctcgccggtggcggatgtgaacgggaacttctttgacagccgccaggcgccgaaaaatatgccacaagacgcagatgcaaatggagcttatcacattggtctgaaaggacttatgctcctgggccgtattaaaaataaccaggagggtaagaaacttaatctggttattaaaaacgaagaatattttgagttcgttcagaaccgtaacaactaa

SEQ ID NO: 47 - DNA, 2235 bp, Pseudomonas aeruginosa
atggacgcggaggctagcgatactcacttttttgctcactccaccttaaaggcagatcgcagcgattggcagcctctggtcgagcatctacaggctgttgcccgtttggcaggagagaaggctgccttcttcggcggcggtgaattagctgctcttgctggtctgttgcatgacttgggtaaatacactgacgagtttcagcggcgtattgcgggtgatgccatccgtgtcgatcactctactcgcggggccatactggcggtagaacgctatggcgcgctaggtcaattgctagcctacggcatcgctggccaccatgccgggttggccaatggccgcgaggctggtgagcgaactgccttggtcgaccgcctgaaaggggttgggctgccacggttattggaggggtggtgcgtggaaatcgtgctacccgagcgccttcaaccaccgccactaaaagcgcgcctggaaagaggtttctttcagttggcctttcttggccggatgctcttttcctgcttggttgatgcggattatctagataccgaagccttctaccaccgcgtcgaaggacggcgctcccttcgcgagcaagcgcggccgaccttggccgagttacgcgcagcccttgatcggcatctgactgagttcaagggagatacgccggtcaaccgcgttcgcggggagatattggccggcgtgcgcggcaaggcgagcgaacttcccgggctgttttctctcacagtgcccacaggaggcggcaagaccctggcctctctggctttcgccctggatcacgctctagctcatgggctgcgccgggtgatctacgtgattcccttcactagcatcgtcgagcagaacgctgcggtattccgtcgtgcactcggggccttaggcgaagaggcggtgctggagcatcacagcgccttcgttgatgaccgccggcagagcctggaggccaagaagaaactgaacctagcgatggagaactgggacgcgcctatcgtggtgaccactgcagtgcagttcttcgaaagcctgtttgccgaccgtccagcccagtgccgcaagctacacaacatcgccggcagcgtggtgattcttgacgaggcacagaccctaccgctcaagctgttgcggccctgcgttgccgcccttgatgaactggcgctcaactaccgttgtagcccagttctctgtactgccacgcagccagcgcttcaatcgccggatttcatcggtgggctgcaggacgtacgtgagctggcgcccgagccgcagcggctgttccgggagttggtgcgggtacgaatacggacattgggcccgctcgaagatgcggccttgactgagcagatcgccaggcgtgaacaagtgctgtgcatcgtcaacaatcgacgccaggcccgtgcgctctatgagtcgcttgccgagttgcccggtgcccgccatctcaccaccctgatgtgcgccaagcaccgtagcagcgtgctggccgaggtgcgccagatgctcaaaaagggggagccctgtcgcctggtggccacctcgctgatcgaggccggtgtggatgtggattttcccgtggtactgcgtgccgaggctggattggattccatcgcccaggccgcgggacgctgcaatcgcgaaggcaagcggccgctggccgaaagcgaggtgctggtgttcgccgcggccaattctgactgggcgccacccgaggaactcaagcagttcgcccaggccgcccgcgaagtgatgcgcctgcacccggatgattgcctgtccatggcggccatcgagcggtattttcgcatactgtactggcagaagggcgcggaggagttggatgcgggtaacctgctcggcctgattgagagaggccggctcgatggcctgccctacgagactttggccaccaagttccgcatgatcgacagccttcaactgccggtgatcatcccatttgatgacgaggccagagcagccctgcgcgagctggagttcgccgacggctgcgccgccatcgcccgtcgcctgcagccatatctggtgcagatgccacgcaagggttatcaggcattgcgggaagccggtgcgatccaggcggcggcaggtacgcgttatggtgagcagtttatggcgttggtcaaccctgatctgtatcaccaccaattcgggttgcactgggataatccggcctttgtcagcagcgagcggctatgttggtag

SEQ ID NO: 48 - DNA, 675 bp, Pseudomonas aeruginosa
atggcctacggaattcgcttaatggtctggggcgagcgtgcctgcttcacccgcccggaaatgaaggtggaacgcgtctcttacgatgcgatcacgccgtccgccgcgcgcggcattctcgaggctatccactggaagccggcgattcgctgggtggtggatcgcattcaagtgcttaagccgatccgcttcgaatccatccggcgcaacgaggtcggcggcaagctgtccgctgtcagcgtcggtaaggcaatgaaggccgggcgtactaatggtctggtgaatctggtcgaggaggatcgccagcagcgcgcgactactctgctgcgcgatgtctcctatgtcatcgaggcgcatttcgagatgactgacagggctggcgccgacgatacggtgggcaagcatctggatatcttcaaccgtcgcgcacggaaggggcagtgcttccatacaccctgcctaggcgtgcgcgagtttccggccagttttcggttgctggaagagggcagtgccgagcctgaagtcgatgcctttctgcgcggcgagcgtgatctgggctggatgctgcatgacattgacttcgccgatggcatgaccccgcacttcttccgtgccctgatgcgcgatgggctgatcgaggtgccggccttcagggcggcagaggacaaggcatga

SEQ ID NO: 49 - DNA, 1764 bp, Pseudomonas aeruginosa
atgatcctttcggccctcaatgactattatcagcgactgctggagcggggtgaagcgaatatctcacccttcggctacagccaagaaaagatcagttacgccctgctgctgtccgcacaaggagagttgctggacgtgcaggacattcgcttgctctctggcaagaagcctcaacccaggcttatgagtgtgccgcagccggagaagcgcacctcgggcatcaagtccaacgtactgtgggacaagaccagctatgtgctgggtgttagtgccaagggcggagagcgtactcagcaggagcacgagtccttcaagacgctgcaccggcagatcttggttggggaaggcgaccccggtctgcaggccttgctccagttcctcgactgttggcagccggagcagttcaagcccccgctgttcagcgaagcaatgctcgacagcaacttagtgttccgcctagacggccaacaacgctatctgcacgagactccggcggccctggcgttgcgtacccggctgttggccgacggcgacagccgcgaggggctgtgcctagtctgcggccaacgtcagccgttggcgcgcctgcatccagcggtcaagggcgtcaatggtgcccagagttcgggggcttccatcgtctccttcaacctcgacgctttttcctcctacggcaagagccagggggaaaatgctccggtctccgaacaggccgcctttgcctacaccacggtgctcaaccatttgttgcgtcgcgacgagcacaaccgccagcgcctgcagattggcgacgcgagtgtggtgttctgggcgcaggcggatactcctgctcaggtggccgccgccgagtcgaccttctggaacctgctggagccacccgcagatgatggtcaggaagcggaaaagctgcgcggcgtgctggatgctgtggccacggggcggcccttgcatgagctcgactcgctaatggaggaaggtacccgcatttttgtgttagggctggcgcccaatacctcgcgactgtccattcggttctgggcagtcgatagccttgcggtattcacccagcatctggccgagcatttccgggatatgcaccttgagcctctgccctggaagacggagccggccatctggcgcttgctctatgctaccgcgcccagtcgtgacggcagagccaagaccgaagacgtactcccacaactggccggtgaaatgacccgcgccatcctgaccggcagccgctatccgcgcagtttgctagccaacctgatcatgcgcatgcgtgccgacggcgacgtctctggcatacgcgtcgcgctgtgcaaggccgtgctcgctcgcgaggcacgcctgagcggcaaaattcaccaagaggagctacctatgagtctcgacaaggacgccagcaaccccggctatcgcttggggaggctgttcgccgtgttggaaggcgcccagcgcgcagccctgggcgacagggtcaatgccactatccgtgaccgctactacggtgccgcgtccagcacgccagccacggttttcccgatactgctgcgcaacacacaaaaccacttggccaagctgcgcaaggagaagcccggactagcagtgaacctagagcgcgatataggcgaaatcattgacggtatgcagagccaattcccgcgttgcctgcgcctggaggaccagggacgctttgctattggttactaccaacaggcccaggcccgtttcaaccgtggccccgattccgtcgagtaa

SEQ ID NO: 50 - DNA, 870 bp, Pseudomonas aeruginosa
atgaccgccatctccaaccgctacgagttcgtttacctctttgatgtcagcaatggcaatcccaatggcgacccggatgctggcaacatgccgcgtctcgatccggaaaccaaccaggggttggtcactgacgtttgcctcaagcgcaagatccgcaactacgtcagcctggagcaggaaagtgcccccggctatgccatctatatgcaggaaaaatccgtgctgaataaccagcacaaacaggcctacgaggcgctcggtatcgagtcagaggcaaagaaactgcccaaggacgaagccaaggcgcgcgaactgacctcttggatgtgcaagaacttcttcgatgtgcgtgctttcggggcggtgatgaccaccgagattaatgccggccaggtgcgtggaccgatccaactggcattcgccacgtctatcgacccggtattgcctatggaggtatccatcacccgcatggcggtgactaacgaaaaggatttggagaaggaacgcaccatgggacgcaagcacatcgtgccttacggcttgtaccgcgcccatggtttcatctctgccaagttggccgagcgaaccggcttttccgacgacgacttggaactgctatggcgcgctttggccaatatgttcgaacacgaccgctcggcggcacgtggcgagatggcagcgcgcaagttgatcgtcttcaagcatgagcatgccatgggcaatgcacccgcccatgtgctgttcggcagcgttaaggtcgagcgagtcgagggggacgcagttacaccagcacgcggtttccaggattaccgtgtcagcatcgatgcggaagctctgcctcagggcgtgagcgtgcgcgagtacctctag

SEQ ID NO: 51 - protein, 744 aa, Pseudomonas aeruginosa
MDAEASDTHFFAHSTLKADRSDWQPLVEHLQAVARLAGEKAAFFGGGELAALAGLLHDLGKYTDEFQRRIAGDAIRVDHSTRGAILAVERYGALGQLLAYGIAGHHAGLANGREAGERTALVDRLKGVGLPRLLEGWCVEIVLPERLQPPPLKARLERGFFQLAFLGRMLFSCLVDADYLDTEAFYHRVEGRRSLREQARPTLAELRAALDRHLTEFKGDTPVNRVRGEILAGVRGKASELPGLFSLTVPTGGGKTLASLAFALDHALAHGLRRVIYVIPFTSIVEQNAAVFRRALGALGEEAVLEHHSAFVDDRRQSLEAKKKLNLAMENWDAPIVVTTAVQFFESLFADRPAQCRKLHNIAGSVVILDEAQTLPLKLLRPCVAALDELALNYRCSPVLCTATQPALQSPDFIGGLQDVRELAPEPQRLFRELVRVRIRTLGPLEDAALTEQIARREQVLCIVNNRRQARALYESLAELPGARHLTTLMCAKHRSSVLAEVRQMLKKGEPCRLVATSLIEAGVDVDFPVVLRAEAGLDSIAQAAGRCNREGKRPLAESEVLVFAAANSDWAPPEELKQFAQAAREVMRLHPDDCLSMAAIERYFRILYWQKGAEELDAGNLLGLIERGRLDGLPYETLATKFRMIDSLQLPVIIPFDDEARAALRELEFADGCAAIARRLQPYLVQMPRKGYQALREAGAIQAAAGTRYGEQFMALVNPDLYHHQFGLHWDNPAFVSSERLCW

SEQ ID NO: 52 - protein, 224 aa, Pseudomonas aeruginosa
MAYGIRLMVWGERACFTRPEMKVERVSYDAITPSAARGILEAIHWKPAIRWVVDRIQVLKPIRFESIRRNEVGGKLSAVSVGKAMKAGRTNGLVNLVEEDRQQRATTLLRDVSYVIEAHFEMTDRAGADDTVGKHLDIFNRRARKGQCFHTPCLGVREFPASFRLLEEGSAEPEVDAFLRGERDLGWMLHDIDFADGMTPHFFRALMRDGLIEVPAFRAAEDKA

SEQ ID NO: 53 - protein, 587 aa, Pseudomonas aeruginosa
MILSALNDYYQRLLERGEANISPFGYSQEKISYALLLSAQGELLDVQDIRLLSGKKPQPRLMSVPQPEKRTSGIKSNVLWDKTSYVLGVSAKGGERTQQEHESFKTLHRQILVGEGDPGLQALLQFLDCWQPEQFKPPLFSEAMLDSNLVFRLDGQQRYLHETPAALALRTRLLADGDSREGLCLVCGQRQPLARLHPAVKGVNGAQSSGASIVSFNLDAFSSYGKSQGENAPVSEQAAFAYTTVLNHLLRRDEHNRQRLQIGDASVVFWAQADTPAQVAAAESTFWNLLEPPADDGQEAEKLRGVLDAVATGRPLHELDSLMEEGTRIFVLGLAPNTSRLSIRFWAVDSLAVFTQHLAEHFRDMHLEPLPWKTEPAIWRLLYATAPSRDGRAKTEDVLPQLAGEMTRAILTGSRYPRSLLANLIMRMRADGDVSGIRVALCKAVLAREARLSGKIHQEELPMSLDKDASNPGYRLGRLFAVLEGAQRAALGDRVNATIRDRYYGAASSTPATVFPILLRNTQNHLAKLRKEKPGLAVNLERDIGEIIDGMQSQFPRCLRLEDQGRFAIGYYQQAQARFNRGPDSVE

SEQ ID NO: 54 - protein, 289 aa, Pseudomonas aeruginosa
MTAISNRYEFVYLFDVSNGNPNGDPDAGNMPRLDPETNQGLVTDVCLKRKIRNYVSLEQESAPGYAIYMQEKSVLNNQHKQAYEALGIESEAKKLPKDEAKARELTSWMCKNFFDVRAFGAVMTTEINAGQVRGPIQLAFATSIDPVLPMEVSITRMAVTNEKDLEKERTMGRKHIVPYGLYRAHGFISAKLAERTGFSDDDLELLWRALANMFEHDRSAARGEMAARKLIVFKHEHAMGNAPAHVLFGSVKVERVEGDAVTPARGFQDYRVSIDAEALPQGVSVREYL

SEQ ID NO: 55 - DNA, 177 bp, Artificial Sequence
  Description of Artificial Sequence Synthetic polynucleotide
gaaaattattttaaatttcctctagtcaggccggaataactccctataatgcgacaccagtcgcgccccgcacgggcgcgtggattgaaacatttatcacaaaaggattgttcgatgtccaacaagtcgcgccccgcacgggcgcgtggattgaaacgcactcccgttctggataat

SEQ ID NO: 56 - DNA, 327 bp, Artificial Sequence
  Description of Artificial Sequence Synthetic polynucleotide
acccggggaaaattattttaaatttcctctagtcaggccggaataactccctataatgcgacaccagtctaagaactttaaataatttctactgttgtagatcattggtagccatgtttctttcctggtctaagaactttaaataatttctactgttgtagatatactggaggagtcataagaattcggtctaagaactttaaataatttctactgttgtagatcggcgaccagtgccgtagtattgatgtctaagaactttaaataatttctactgttgtagattcctcgctcactgactcgcttcgagctcggtacctgacagct